SEQUENCE LISTING (40 sequences)

SEQ ID NO: 1 - DNA, 2781 bp, Homo sapiens
  misc_feature (546)..(546): n is a, c, g, or t
cgcggccgcgctgcccctggcttcgcggaagccctgagtagcgcagcgccctcgccgcacgcaaggctgcagtcccgctccaggccagagtcccaggagagcgtcctccgcgctcacaggcgcctttgtcttcccgcttcccccttcctttttcaaaagccaagaggtaattatttggtctttgtgcaaggcaaacctctccagatgccacttccaaatataggctctcattaacaccagaggctggcctggtgtggtgcagggcggcccttccttctcctggcggacactgtgtccccgcgcgctggcgctgcaccacatctggaagccaggcgggcagggcagagaccccggctcctgcgcccctcctagctcccagagagcgtggatcgcgggcggggctcaccgagcgaggttacctctcttgaaaatacttaaacactttttttcctctccactgaaatctcaaaaaacagcccattttgaaccagaataatttagtctgacaacagattcttcctctgttcacagctgtcccagaggagngagctgaaatctgaacctctcagctgtgattggatctttcttgcaaaagagaggaaaaaaaaaccctcccagccaaaacgggctcagttcgtaaaggagccgggtgacttcagaggcgccggcccgtccgtctgccgcacctgagcacggcccctgcccgagcctggcccgccgcgatgctgtagggaccgccgtgtcctcccgccggaccgttatccgcgccgggcgcccgccagacccgctggcaagatgccgcgctccttcctggtcaagaagcatttcaacgcctccaaaaagccaaactacagcgaactggacacacatacagtgattatttccccgtatctctatgagagttactccatgcctgtcataccacaaccagagatcctcagctcaggagcatacagccccatcactgtgtggactaccgctgctccattccacgcccagctacccaatggcctctctcctctttccggatactcctcatctttggggcgagtgagtccccctcctccatctgacacctcctccaaggaccacagtggctcagaaagccccattagtgatgaagaggaaagactacagtccaagctttcagacccccatgccattgaagctgaaaagtttcagtgcaatttatgcaataagacctattcaactttttctgggctggccaaacataagcagctgcactgcgatgcccagtctagaaaatctttcagctgtaaatactgtgacaaggaatatgtgagcctgggcgccctgaagatgcatattcggacccacacattaccttgtgtttgcaagatctgcggcaaggcgttttccagaccctggttgcttcaaggacacattagaactcacacgggggagaagcctttttcttgccctcactgcaacagagcatttgcagacaggtcaaatctgagggctcatctgcagacccattctgatgtaaagaaataccagtgcaaaaactgctccaaaaccttctccagaatgtctctcctgcacaaacatgaggaatctggctgctgtgtagcacactgagtgacgcaatcaatgtttactcgaacagaatgcatttcttcactccgaagccaaatgacaaataaagtccaaaggcattttctcctgtgctgaccaaccaaataatatgtatagacacacacacatatgcacacacacacacacacacccacagagagagagctgcaagagcatggaattcatgtgtttaaagataatcctttccatgtgaagtttaaaattactatatatttgctgatggctagattgagagaataaaagacagtaacctttctcttcaaagataaaatgaaaagcacattgcatcttttcttcctaaaaaaatgcaaagatttacattgctgccaaatcatttcaactgaaaagaacagtattgctttgtaatagagtctgtaataggatttcccataggaagagatctgccagacgcgaactcaggtgccttaaaaagtattccaagtttactccattacatgtcggttgtctggttgccattgttgaactaaagcctttttttgattacctgtagtgctttaaagtatatttttaaaagggaggaaaaaaataacaagaacaaaacacaggagaatgtattaaaagtatttttgttttgttttgtttttgccaattaacagtatgtgccttgggggaggagggaaagattagctttgaacattcctggcgcatgctccattgtcttactattttaaaacattttaataatttttgaaaattaattaaagatgggaataagtgcaaaagaggattcttacaaattcattaatgtacttaaactatttcaaatgcataccacaaatgcaataatacaataccccttccaagtgcctttttaaattgtatagttgatgagtcaatgtaaatttgtgtttatttttatatgattgaatgagttctgtatgaaactgagatgttgtctatagctatgtctataaacaacctgaagacttgtgaaatcaatgtttcttttttaaaaaacaattttcaagttttttttacaataaacagttttgatttaaaatctcgtttgtatactattttcagagactttacttgcttcatgattagtaccaaaccactgtacaaagaattgtttgttaacaagaaaaaaatgaataatgcttattatgcatctgaagtgttattttatgtgttagatta

SEQ ID NO: 2 - protein, 268 aa, Homo sapiens
MPRSFLVKKHFNASKKPNYSELDTHTVIISPYLYESYSMPVIPQPEILSSGAYSPITVWTTAAPFHAQLPNGLSPLSGYSSSLGRVSPPPPSDTSSKDHSGSESPISDEEERLQSKLSDPHAIEAEKFQCNLCNKTYSTFSGLAKHKQLHCDAQSRKSFSCKYCDKEYVSLGALKMHIRTHTLPCVCKICGKAFSRPWLLQGHIRTHTGEKPFSCPHCNRAFADRSNLRAHLQTHSDVKKYQCKNCSKTFSRMSLLHKHEESGCCVAH

SEQ ID NO: 3 - DNA, 4325 bp, Homo sapiens
catccccttgaccctccaatcacctcgggctcccatttgattggaaggcggcaaaggctttaatctcccccttggtgcagctgcttttgaagtgagtttcctcgccagagccccggctggacacgcagcggctcgcatcgcagagcgcagcgccggcgcggggccgcgagaacgcagcgcaggggagcagcccgaggcggacaccgcgagccgcccggcactcccgcagtccagccggctcctctagcccggccacggctccgctgcgggccacccaggattactcgcgtctggctccaggcgccgagaaggcgcgctgggcgcccgtggccgccgcgccagctcctcctcctcccgctgctcctgctcccggggcgagcgcgcagccccgagcccgccccgcgcctcccggagccctcccccccgctgctcccatgcgcgcgggtgggtcatgagcacagcgccctcgctttctgccctaagaagcagtaagcacagcggcggcggcggcggcggaggcggaggcggcggtgcagaccctgcctggaccagcgcgctctctggaaatagctccggccccggcccaggctcgtccccggccggcagcaccaagccttttgtgcacgccgtgcccccctctgaccccctgcgccaggccaaccgcctgccaatcaaggtgctgaagatgctgacggcacgaactggccacattttgcaccccgagtacctgcagcccctgccttccacgccggtcagccccatcgaggtaaggaccctctctctggatcgcactgggaccactaccctggctgccaccctagggctttctttttctcgggatctgggcggaggtgggggggtcggagtgattcagctcccgaatgggggaagaggctactgcttccgtacctcaaaactagggcggaaaagggggaggaagtggaatggggcgtgcatgctagggagcaaggctgccaatacttgtttctcctttcgatatgaaagcccctaccccgacccaggccccttcactcggcaccgaaggcaggcggaggtctgaaatacggttccaaagtcgccgtccttcgtatccgcagaagccagtgtgtgcacacagcctctgaggcgccagccgcccgagcccttactctgaagaattaaggagtgtttgtggggagggggtacagttctgggtctaggaaccgaaaaccaaaacattttgctctttaaaaatctagttagcgctcagagagggcaggaaagatgctgctgggggtggtggttgggcggggggagcaatctgctgcctttcccaacggcgagaatgtttgtgagtgggtgttgaagagggggtgccgcctagaattgcgccttggggctgggaggatcttcgtgggctgttgcggagaggcatttgaaccccagaagccaggattctaaagggtttccacttctttctctgtgtgacgctccccccccatcgtctgaccccgcagctcgatgccaagaagagcccgctggcgctgttggcgcaaacatgttcgcagatcgggaagcccgacccctcgccctcctccaaactctcctcggttgcctccaacgggggcggcgcgggcggtgccggcggcggtgctgcgggcgacaaggacaccaaatcgggccccctgaagctgagcgacatcggcgtggaggacaagtcgagtttcaagccgtactccaaacccggctcggataagaaggagccgggaggcggcggtggaggcggtggcggtggcgggggcggcggcgggggtgtttcgtcggagaagtcgggattccgggtaccgagcgccacctgccagccattcacgcccaggacaggcagcccgagctccagcgcctcggcctgctcgccgggaggtatgctgtcctcggccgggggtgccccggagggcaaggacgacaagaaagacaccgacgtgggcggcggtggcaagggcaccgggggcgcctcggccgaagggggacccacggggctggcacacggccggattagctgcggcggcgggattaatgtggatgtgaaccagcatccggatgggggcccgggaggcaaggctctgggctcggactgcggcggttcatcgggctccagctccggctccggccccagcgcgcccacctcctcctcagtgttgggctctgggctggtggctcccgtgtcaccctacaagccgggccagacagtgttccctctgcctcccgcgggtatgacctacccaggcagcctggccggggcctacgccggctacccgccccagttcctgccacacggcgtggcacttgaccccaccaagccgggcagcctggtgggggcgcagctggcggcggccgcggccgggtctctgggctgcagtaagccggccggctccagccctttggccggagcgtctccgccgtccgtgatgacagccagtttgtgccgggacccttactgcctcagctaccactgcgctagccacctggcaggggcggcggccgccagcgcttcttgcgcacatgatccggctgctgcggctgcggcgctgaagtccggatacccgctggtgtaccccacgcacccgctgcacggtgtgcactcctcgctaacggccgccgcggctgctggcgccacaccgccctccctggccggccaccccctctacccctacggctttatgctccctaacgacccactcccccacatctgcaactgggtgtcggccaacgggccgtgcgacaagcgcttcgccacgtccgaagagctgctgagccacttgcggacccatacggcatttcccgggacagacaaactgctgtcgggctaccccagctcgtcgtctctggccagcgctgccgcggccgccatggcttgccacatgcacatccccacctcgggcgcaccgggcagccctgggacgctggcgctgcgcagcccccaccacgcgctgggactcagcagccgctaccacccctactccaagagcccgcttcccacgcctggcgcccccgtgccggtgcccgccgccaccggaccgtactactccccctacgccctctacggacagagactgaccaccgcctcggcgctggggtatcagtgagggcggccgggagggcgagcgagggagaggagggagagggggaggggaggagtccagggagaggcgggatcacggcccaggctgctgacacccgcgcgtggggaggactcgggccacgaaaggaaagaaatgtataccgtatctatctacccgacagcagcgaccgagacccggtgggacactccccttctccccactttcacctccccacccaaactttataaaagttgaaaaaatatcatttgactttttatagaaaaaaaaaggaaaaaataattgagaaagtgttcatctgaggactgcatcggtggacactggtatttatttatgttagctccaagcggaccggtggttcaaaagtgcattatttagtttgagctctgtaggtaaaaaggaggtgggaaaaattttaaaacttgagggtaaaaatgtggaaaacaaaccctcccatcccttgtagattataaataaaagcaaaaccgccacagaactagaggtcttctctttaatgttactttaaaattgctatgattgtattgtacgttatttaatgtctgattgaaacacaaatttacatgcatgtttgttacaaaaaaaatgaaaaaaaaagtcacaatttgtcagctctgatttcaaattgcaattatttttaaggtgtataccatcgaagagaatgggtatttttttgtatgtattctggaagaaaacaacaaaaaaaaaaagaaaaagaaaaaattctattccaaaacctcatttgccttattttgttctttaaaaggaacacttaactatttttaatttttaagtccacccgctgagaaggggacaaggtttacgtcatgtactaaaataatagacaatgtatcgctttaaagattaaaattccgtatatttgatgtaaaaaaaaaaaaaacaaaaaacaaaaaacacttttgtggcgcgcttgagcctggagaaaagtgttagacaacacattgcgtattggggcgcggggccccatgatggataagatgacacaggtgcacaaagcagtgtcccaagaaagacccgcggacaagacacagaggggtaggggaatccacacgaaaaaaaaagccccggggggacaaccagagggttctgagagaaccccaaagggatctcagagggcgacaagagggcgtatagacaaagggacacagaggagaagtgagaacaattgcc

SEQ ID NO: 4 - protein, 619 aa, Homo sapiens
MLLGVVVGRGEQSAAFPNGENVCEWVLKRGCRLELRLGAGRIFVGCCGEAFEPQKPGFYKGFHFFLCVTLPPHRLTPQLDAKKSPLALLAQTCSQIGKPDPSPSSKLSSVASNGGGAGGAGGGAAGDKDTKSGPLKLSDIGVEDKSSFKPYSKPGSDKKEPGGGGGGGGGGGGGGGGVSSEKSGFRVPSATCQPFTPRTGSPSSSASACSPGGMLSSAGGAPEGKDDKKDTDVGGGGKGTGGASAEGGPTGLAHGRISCGGGINVDVNQHPDGGPGGKALGSDCGGSSGSSSGSGPSAPTSSSVLGSGLVAPVSPYKPGQTVFPLPPAGMTYPGSLAGAYAGYPPQFLPHGVALDPTKPGSLVGAQLAAAAAGSLGCSKPAGSSPLAGASPPSVMTASLCRDPYCLSYHCASHLAGAAAASASCAHDPAAAAAALKSGYPLVYPTHPLHGVHSSLTAAAAAGATPPSLAGHPLYPYGFMLPNDPLPHICNWVSANGPCDKRFATSEELLSHLRTHTAFPGTDKLLSGYPSSSSLASAAAAAMACHMHIPTSGAPGSPGTLALRSPHHALGLSSRYHPYSKSPLPTPGAPVPVPAATGPYYSPYALYGQRLTTASALGYQ

SEQ ID NO: 5 - DNA, 2483 bp, Homo sapiens
gagagcagaccaggcccggtggagaattaggtgctgctgggagctcctgcctcccacaggattccagctgcagggagcctcagggactctgggccgcacggagttgggggcattccccagagagcgtcgccatggtctgcagggagcagttatcaaagaatcaggtcaagtgggtgtttgccggcattacctgtgtgtctgtggtggtcattgccgcaatagtccttgccatcaccctgcggcggccaggctgtgagctggaggcctgcagccctgatgccgacatgctggactacctgctgagcctgggccagatcagccggcgagatgccttggaggtcacctggtaccacgcagccaacagcaagaaagccatgacagctgccctgaacagcaacatcacagtcctggaggctgacgtcaatgtagaagggctcggcacagccaatgagacaggagttcccatcatggcacacccccccactatctacagtgacaacacactggagcagtggctggacgctgtgctgggctcttcccaaaagggcatcaaactggacttcaagaacatcaaggcagtgggcccctccctggacctcctgcggcagctgacagaggaaggcaaagtccggcggcccatatggatcaacgctgacatcttaaagggccccaacatgctcatctcaactgaggtcaatgccacacagttcctggccctggtccaggagaagtatcccaaggctaccctatctccaggctggaccaccttctacatgtccacgtccccaaacaggacgtacacccaagccatggtggagaagatgcacgagctggtgggaggagtgccccagagggtcaccttccctgtacggtcttccatggtgcgggctgcctggccccacttcagctggctgctgagccaatctgagaggtacagcctgacgctgtggcaggctgcctcggaccccatgtcggtggaagatctgctctacgtccgggataacactgctgtccaccaagtctactatgacatctttgagcctctcctgtcacagttcaagcagctggccttgaatgccacacggaaaccaatgtactacacgggaggcagcctgatccctcttctccagctgcctggggatgacggtctgaatgtggagtggctggttcctgacgtccagggcagcggtaaaacagcaacaatgaccctcccagacacagaaggcatgatcctgctgaacactggcctcgagggaactgtggctgaaaaccccgtgcccattgttcatactccaagtggcaacatcctgacgctggagtcctgcctgcagcagctggccacacatcccggacactggggcatccatttgcaaatagtggagcccgcagccctccggccatccctggccttgctggcacgcctctccagccttggcctcttgcattggcctgtgtgggttggggccaaaatctcccacgggagtttttcggtccccggccatgtggctggcagagagctgcttacagctgtggctgaggtcttcccccacgtgactgtggcaccaggctggcctgaggaggtgctgggcagtggctacagggaacagctgctcacagatatgctagagttgtgccaggggctctggcaacctgtgtccttccagatgcaggccatgctgctgggccacagcacagctggagccataggcaggctgctggcatcctccccccgggccaccgtcacagtggagcacaacccagctgggggcgactatgcctctgtgaggacagcattgctggcagctagggctgtggacaggacccgagtctactacaggctaccccagggctaccacaaggacttgctggctcatgttggtagaaactgagcacccaggggtggtgggccagcggacctcagggcggaggcttcccacggggaggcaggaagaaataaaggtctttggctttctccaggcactgtatgtgwgtccttggggacaggatggagtgggagtgggcatgatgtggccactgagggcatctagagggtctggaggctgggggccagatcattccggttgtccaagagaaactgctcacaagccttgaaggtggtgtagaactcagaggagaggccggccacgttggtggtcacatagttgagaacacctggggtggcctggttgtagtaggataccttggtcagctggtccccctcgcgccagaggcagaagcctgagcagagggtctctccgcgtctgtactctggcgtctctcggtgtgtgggcagcgtgaccgacctcagcgcgatgacataggggtccccattgtcacaaggcttccgcctcgaggccaggatcacgaagtcctggggctttgtgtgacctccgagggcagggctggtgacgtggtagatggcgtcgtcctcgtctacctgctgcactagctccacgctccggtagtgcttgtcccactctggc

SEQ ID NO: 6 - protein, 629 aa, Homo sapiens
AEQTRPGGELGAAGSSCLPQDSSCREPQGLWAARSWGHSPESVAMVCREQLSKNQVKWVFAGITCVSVVVIAAIVLAITLRRPGCELEACSPDADMLDYLLSLGQISRRDALEVTWYHAANSKKAMTAALNSNITVLEADVNVEGLGTANETGVPIMAHPPTIYSDNTLEQWLDAVLGSSQKGIKLDFKNIKAVGPSLDLLRQLTEEGKVRRPIWINADILKGPNMLISTEVNATQFLALVQEKYPKATLSPGWTTFYMSTSPNRTYTQAMVEKMHELVGGVPQRVTFPVRSSMVRAAWPHFSWLLSQSERYSLTLWQAASDPMSVEDLLYVRDNTAVHQVYYDIFEPLLSQFKQLALNATRKPMYYTGGSLIPLLQLPGDDGLNVEWLVPDVQGSGKTATMTLPDTEGMILLNTGLEGTVAENPVPIVHTPSGNILTLESCLQQLATHPGHWGIHLQIVEPAALRPSLALLARLSSLGLLHWPVWVGAKISHGSFSVPGHVAGRELLTAVAEVFPHVTVAPGWPEEVLGSGYREQLLTDMLELCQGLWQPVSFQMQAMLLGHSTAGAIGRLLASSPRATVTVEHNPAGGDYASVRTALLAARAVDRTRVYYRLPQGYHKDLLAHVGRN

SEQ ID NO: 7 - DNA, 3037 bp, Homo sapiens
ctggggctggattgagctgaccacaggccacaccagactcctctctgctcctgaggaagacagggcagcccggcgccacccgctcggccctcacgaagatgctccctggagcctggctgctctggacctccctcctgctcctggccaggcctgcccagccctgtcccatgggttgtgactgcttcgtccaggaggtgttctgctcagatgaggagcttgccaccgtcccgctggacatcccgccatatacgaaaaacatcatctttgtggagacctcgttcaccacattggaaaccagagcttttggcagtaaccccaacttgaccaaggtggtcttcctcaacactcagctctgccagtttaggccggatgcctttggggggctgcccaggctggaggacctggaggtcacaggcagtagcttcttgaacctcagcaccaacatcttctccaacctgacctcgctgggcaagctcaccctcaacttcaacatgctggaggctctgcccgagggtcttttccagcacctggctgccctggagtccctccacctgcaggggaaccagctccaggccctgcccaggaggctcttccagcctctgacccatctgaagacactcaacctggcccagaacctcctggcccagctcccggaggagctgttccacccactcaccagcctgcagaccctgaagctgagcaacaacgcgctctctggtctcccccagggtgtgtttggcaaactgggcagcctgcaggagctcttcctggacagcaacaacatctcggagctgccccctcaggtgttctcccagctcttctgcctagagaggctgtggctgcaacgcaacgccatcacgcacctgccgctctccatctttgcctccctgggtaatctgacctttctgagcttgcagtggaacatgcttcgggtcctgcctgccggcctctttgcccacaccccatgcctggttggcctgtctctgacccataaccagctggagactgtcgctgagggcacctttgcccacctgtccaacctgcgttccctcatgctctcatacaatgccattacccacctcccagctggcatcttcagagacctggaggagttggtcaaactctacctgggcagcaacaaccttacggcgctgcacccagccctcttccagaacctgtccaagctggagctgctcagcctctccaagaaccagctgaccacacttccggagggcatcttcgacaccaactacaacctgttcaacctggccctgcacggtaacccctggcagtgcgactgccacctggcctacctcttcaactggctgcagcagtacaccgatcggctcctgaacatccagacctactgcgctggccctgcctacctcaaaggccaggtggtgcccgccttgaatgagaagcagctggtgtgtcccgtcacccgggaccacttgggcttccaggtcacgtggccggacgaaagcaaggcagggggcagctgggatctggctgtgcaggaaagggcagcccggagccagtgcacctacagcaaccccgagggcaccgtggtgctcgcctgtgaccaggcccagtgtcgctggctgaacgtccagctctctcctcggcagggctccctgggactgcagtacaatgctagtcaggagtgggacctgaggtcgagctgcggttctctgcggctcaccgtgtctatcgaggctcgggcagcagggccctagtagcagcgcatacaggagctggggaagggggcctctggggcctgaccaggcgacaggtaggggcggaggggagctgagtctccgaagccttggcttttcacatgcaagggacagggttacatccccaaggtgagggggtggagtctggtctgctccactaaccagggtctcctcctcctcttccttcatcgcttctcctggagtgtgcggcctaacaaggccatccttatgctttgcaaagcaccctcaaaagctgcaccacagcctggagaataaaatatcctcagccctgatgcctccccattatgtaacacccaaccgctctcacctacaccctgaggtctattcactgcatcccagtgatacaaagtggaggccactgccttctgacatctggctcaaaagcccagtgtctgtttccatttatttccctggaatttcatttaaaattggtatagagaaaaaaaggatgtgacagaagcagagatgaccagaaagcacaggggcagggttctgactggcgtgtgggagaccctgtggccggcacccacctccacacgaggactaagctctgatttttttatcttgcccaaattcctacctaaggggtctagggagtcgcgccttacaaatcataaattctcatcagatgggttttatttgaccctgtatatcatgacttatttttaatctgactatggcataacattacaagacgaggcaaaaatatttaacccccaaatatatttcctttgccctaccttgaacttgccctgcagagtctcttgtgaggagaatccacatcctataaaggaagcccctttcccctttgttttccttcctttctttccagtccaggagatcatcaactaagagccaggcaccccttttaagtcgataagaaacagtttacaacctgctctctctctctctgaagtctgctgagagcttcccctgcacaataaaacttggcctccacaatcctttatcttaacctgaacattcctttccattgatcccaggtcttcagctragctcaaccaattgtcaaccagaaaatgtttaaatttacctacagcctggaagcacccacccccgctgcttcgagttgtcctgcctttctgaactcaaccaatgtatttcttaaatgtatttgattgatgcctcattcctccctaaaatgtataaaaccaagctgtacctcgaccaccttgggcacatgttcccaggccctcctgaggtctgtgtcacgggccatggccactcatatttggctcagaataaatctcttcaaawayyyy

SEQ ID NO: 8 - protein, 545 aa, Homo sapiens [sequence omitted]

SEQ ID NO: 9 - DNA, 1846 bp, Homo sapiens
gcgggccgcgcgcatcccctgcggcggcggcggcggcctcgggcgggtgggggcagcgctcccgctccgcccccggcccccgctgcccgcgtcccctgccaggcccctcctcccggcggcgcggcagagccaggccccagcgctcggccggccgcgagcccgccggccggggacgagcgtcgcagctcatgctgatcgctgtcctcctcctccccctcaggcggcgctggcggcggccctgggacccgcggaagccggcatgctggagaagctggagttcgaggacgaagcagtagaagactcagaaagtggtgtttacatgcgattcatgaggtcacacaagtgttatgacatcgttccaaccagttcaaagcttgttgtctttgatactacattacaagttaaaaaggccttctttgctttggtagccaacggtgtccgagcagcgccactgtgggagagtaaaaaacaaagttttgtaggaatgctaacaattacagatttcataaatatactacatagatactataaatcacctatggtacagatttatgaattagaggaacataaaattgaaacatggagggagctttatttacaagaaacatttaagcctttagtgaatatatctccagatgcaagcctcttcgatgctgtatactccttgatcaaaaataaaatccacagattgcccgttattgaccctatcagtgggaatgcactttatatacttacccacaaaagaatcctcaagttcctccagctttttatgtctgatatgccaaagcctgccttcatgaagcagaacctggatgagcttggaataggaacgtaccacaacattgccttcatacatccagacactcccatcatcaaagccttgaacatatttgtggaaagacgaatatcagctctgcctgttgtggatgagtcaggaaaagttgtagatatttattccaaatttgatgtaattaatcttgctgctgagaaaacatacaataacctagatatcacggtgacccaggcccttcagcaccgttcacagtattttgaaggtgttgtgaagtgcaataagctggaaatactggagaccatcgtggacagaatagtaagagctgaggtccatcggctggtggtggtaaatgaagcagatagtattgtgggtattatttccctgtcggacattctgcaagccctgatcctcacaccagcaggtgccaaacaaaaggagacagaaacggagtgaccgccgtgaatgtagacgccctaggaggagaacttgaacaaagtctctgggtcacgttttgcctcatgaacactggctgcaagtggttaagaatgtatatcagggtttaacgataggtatttcttccagtgatgttgaaattaagcttaaaaaagaaagattttatgtgcttgaagattcaggcttgcattaaaagactgttttcagacctttgtctgaaggattttaaatgctgtatgtcattaaagtgcactgtgtcctgaagttttcattatttttcatttcaaagaattcactggtatggaacaggtgatgtggcataaggtgagtgcacggtatgttcagatcacagtgccttatgtccgaatacagcaatatgtcaccgccgcagccggggcgcacgcgtgtgaaacaacaccgagcttgaatgtggaagtctttgaaccttttaccaaatcagtttgttttctttagatttgtcaaaaagttgtaatttgaatataaataattactttaaaattgtaatgacacttttacacgtaagtgttttgttctgggctgccttgggcggctgccattgggcacggctgccattggtgt

SEQ ID NO: 10 - protein, 414 aa, Homo sapiens
RAARIPCGGGGGLGRVGAALPLRPRPPLPASPARPLLPAARQSQAPALGRPRARRPGTSVAAHADRCPPPPPQAALAAALGPAEAGMLEKLEFEDEAVEDSESGVYMRFMRSHKCYDIVPTSSKLVVFDTTLQVKKAFFALVANGVRAAPLWESKKQSFVGMLTITDFINILHRYYKSPMVQIYELEEHKIETWRELYLQETFKPLVNISPDASLFDAVYSLIKNKIHRLPVIDPISGNALYILTHKRILKFLQLFMSDMPKPAFMKQNLDELGIGTYHNIAFIHPDTPIIKALNIFVERRISALPVVDESGKVVDIYSKFDVINLAAEKTYNNLDITVTQALQHRSQYFEGVVKCNKLEILETIVDRIVRAEVHRLVVVNEADSIVGIISLSDILQALILTPAGAKQKETETE

SEQ ID NO: 11 - DNA, 2813 bp, Homo sapiens
gaagccttcaggcagcctcaactcagccccaagccactgctctcccatcccagtccctggaaatccacccacttggcccagctcaccccaactccaacccactgggacccagtctccaggggcctgactgtgggcggcagccactcctgagtgagcaaaggttcctccgcggtgctctcccgtccagagccctgctgatggggaagtccgaaggcccagtggggatggtggagagcgccggccgtgcagggcagaagcgcccggggttcctggagggggggctgctgctgctgctgctgctggtgaccgctgccctggtggccttgggtgtcctctacgccgaccgcagagggatcccagaggcccaagaggtgagcgaggtctgcaccacccctggctgcgtgatagcagctgccaggatcctccagaacatggacccgaccacggaaccgtgtgacgacttctaccagtttgcatgcggaggctggctgcggcgccacgtgatccctgagaccaactcaagatacagcatctttgacgtcctccgcgacgagctggaggtcatcctcaaagcggtgctggagaattcgactgccaaggaccggccggctgtggagaaggccaggacgctgtaccgctcctgcatgaaccagagtgtgatagagaagcgaggctctcagcccctgctggacatcttggaggtggtgggaggctggccggtggcgatggacaggtggaacgagaccgtaggactcgagtgggagctggagcggcagctggcgctgatgaactcacagttcaacaggcgcgtcctcatcgacctcttcatctggaacgacgaccagaactccagccggcacatcatctacatagaccagcccaccttgggcatgccctcccgagagtactacttcaacggcggcagcaaccggaaggtgcgggaagcctacctgcagttcatggtgtcagtggccacgttgctgcgggaggatgcaaacctgcccagggacagctgcctggtgcaggaggacatggtgcaggtgctggagctggagacacagctggccaaggccacggtaccccaggaggagagacacgacgtcatcgccttgtaccaccggatgggactggaggagctgcaaagccartttggcctgaagggatttaactggactctgttcatacaaactgtgctatcctctgtcaaaatcaagctgctgccagatgaggaagtggtggtctatggcatcccctacctgcagaaccttgaaaacatcatcgacacctactcagccaggaccatacagaactacctggtctggcgcctggtgctggaccgcattggtagcctaagccagagattcaaggacacacgagtgaactaccgcaaggcgctgtttggcacaatggtggaggaggtgcgctggcgtgaatgtgtgggctacgtcaacagcaacatggagaacgccgtgggctccctctacgtcagggaggcgttccctggagacagcaagagcatggtcagagaactcattgacaaggtgcggacagtgtttgtggagacgctggacgagctgggctggatggacgaggagtccaagaagaaggcgcaggagaaggccatgagcatccgggagcagatcgggcaccctgactacatcctggaggagatgaacaggcgcctggacgaggagtactccaatctgaacttctcagaggacctgtactttgagaacagtctgcagaacctcaaggtgggcgcccagcggagcctcaggaagcttcgggaaaaggtggacccaaatctctggatcatcggggcggcggtggtcaatgcgttctactccccaaaccgaaaccagattgtattccctgccgggatcctccagccccccttcttcagcaaggagcagccacaggccttgaactttggaggcattgggatggtgatcgggcacgagatcacgcacggctttgacgacaatggccggaacttcgacaagaatggcaacatgatggattggtggagtaacttctccacccagcacttccgggagcagtcagagtgcatgatctaccagtacggcaactactcctgggacctggcagacgaacagaacgtgaacggattcaacacccttggggaaaacattgctgacaacggaggggtgcggcaagcctataaggcctacctcaagtggatggcagagggtggcaaggaccagcagctgcccggcctggatctcacccatgagcagctcttcttcatcaactatgcccaggtgtggtgcgggtcctaccggcccgagttcgccatccaatccatcaagacagacgtccacagtcccctgaagtacagggtactggggtcgctgcagaacctggccgccttcgcagacacgttccactgtgcccggggcacccccatgcaccccaaggagcgatgccgcgtgtggtagccaaggccctgccgcgctgtgcggcccacgcccacccgctgctcggaggcatctgtgcgaaggtgcagctagcggcgacccagtgtacgtcccgccccggccaaccatgccaagcctgcctgccaggcctctgcgcctggcctagggtgcagccacctgcctgacacccagggatgagcagtgtccagtgcagtacctggaccggagccccctccacagacacccgcggggctcagtgcccccgtcacagctctgtagagacaatcaactgtgtcctgcccaccctccaaggtgcattgtcttccagtatctacagcttcagacttgagctaagtaaatgcttcaaagaaatca

SEQ ID NO: 12 - protein, 818 aa, Homo sapiens
SLQAASTQPQATALPSQSLEIHPLGPAHPNSNPLGPSLQGPDCGRQPLLSEQRFLRGALPSRALLMGKSEGPVGMVESAGRAGQKRPGFLEGGLLLLLLLVTAALVALGVLYADRRGIPEAQEVSEVCTTPGCVIAAARILQNMDPTTEPCDDFYQFACGGWLRRHVIPETNSRYSIFDVLRDELEVILKAVLENSTAKDRPAVEKARTLYRSCMNQSVIEKRGSQPLLDILEVVGGWPVAMDRWNETVGLEWELERQLALMNSQFNRRVLIDLFIWNDDQNSSRHIIYIDQPTLGMPSREYYFNGGSNRKVREAYLQFMVSVATLLREDANLPRDSCLVQEDMVQVLELETQLAKATVPQEERHDVIALYHRMGLEELQSQFGLKGFNWTLFIQTVLSSVKIKLLPDEEVVVYGIPYLQNLENIIDTYSARTIQNYLVWRLVLDRIGSLSQRFKDTRVNYRKALFGTMVEEVRWRECVGYVNSNMENAVGSLYVREAFPGDSKSMVRELIDKVRTVFVETLDELGWMDEESKKKAQEKAMSIREQIGHPDYILEEMNRRLDEEYSNLNFSEDLYFENSLQNLKVGAQRSLRKLREKVDPNLWIIGAAVVNAFYSPNRNQIVFPAGILQPPFFSKEQPQALNFGGIGMVIGHEITHGFDDNGRNFDKNGNMMDWWSNFSTQHFREQSECMIYQYGNYSWDLADEQNVNGFNTLGENIADNGGVRQAYKAYLKWMAEGGKDQQLPGLDLTHEQLFFINYAQVWCGSYRPEFAIQSIKTDVHSPLKYRVLGSLQNLAAFADTFHCARGTPMHPKERCRVW

SEQ ID NO: 13 - DNA, 3662 bp, Homo sapiens
cggcgcgtcgctgcgccgcacgtgtgcgagcccggccgccggtgagtcggctggagcgcatctggtcctccgcgcggaaagcgctgcttttgcctggccgccctagccgctggctcatccaagtggccttcgccgctctcttgcgtcccaaccagagcgctggccacctcgccgcccagctcacgccgcgcccgcgctcccaggctccgggttttcttaaatgttttcttggagccttaaagatggagatgacagaaatgactggtgtgtcgctgaaacgtggggcactggttgtcgaagataatgacagtggagtcccagttgaagagacaaaaaaacagaagctgtcggaatgcagtctaaccaaaggtcaagatgggctacagaatgactttctgtccatcagtgaagacgtgcctcggcctcctgacactgtcagtactgggaaaggtggaaagaattctgaggctcagttggaagatgaggaagaagaggaggaagatggactttcagaggagtgcgaggaggaggaatcagagagttttgcagacatgatgaagcatggactcactgaggctgacgtaggcatcaccaagtttgtgagttctcatcaagggttctcgggaatcttaaaagaaagatactccgacttcgttgttcatgaaataggaaaagatggacggatcagccatttgaatgacttgtccattccagtggatgaggaggacccttcagaagacatatttacagttttgacagctgaagaaaagcagcgattggaagagctccagctgttcaaaaataaggaaaccagtgttgccattgaggttatcgaggacaccaaagagaaaagaaccatcatccatcaggctatcaaatctctgtttccaggattagagacaaaaacagaggatagggaggggaagaaatacattgtagcctaccacgcagctgggaaaaaggctttggcaagtaggtgtctgtgagcagcttcctcctgccaggcagccgtgagcggcaggccccgctcctcgttagccttgcccgttcgtgttcaagggctactatagtgctgctcagagaacaatattgatcatgtccctccttcagagaggaggtcagaactgcaagatccaagaaaacattcttggccaaaatctaggggaagttactgccacttcgtactatataaggaaaacaaagacaccatggatgctattaatgtactctccaaatacttaagagtcaagccaaatatattctcctacatgggaaccaaagataaaagggctataacagttcaagaaattgctgttctcaaaataactgcacaaagacttgcccacctgaataagtgcttgatgaactttaagctagggaatttcagctatcaaaaaaacccactgaaattgggagagcttcaaggaaaccacttcactgttgttctcagaaatataacaggaactgatgaccaagtacagcaagctatgaactctctcaaggagattggatttattaactactatggaatgcaaagatttggaaccacagctgtccctacgtatcaggttggaagagctatactacaaaattcctggacagaagtcatggatttaatattgaaaccccgctctggagctgaaaagggctacttggttaaatgcagagaagaatgggcaaagaccaaagacccaactgctgccctcagaaaactacctgtcaaaaggtgtgtggaagggcagctgcttcgaggactttcaaaatatggaatgaagaatatagtctctgcatttggcataatacccagaaataatcgcttaatgtatattcatagctaccaaagctatgtgtggaataacatggtaagcaagaggatagaagactatggactaaaacctgttccaggggacctcgttctcaaaggagccacagccacctatattgaggaagatgatgttaataattactctatccatgatgtggtaatgcccttgcctggtttcgatgttatctacccaaagcataaaattcaagaagcctacagggaaatgctcacagctgacaatcttgatattgacaacatgagacacaaaattcgagattattccttgtcaggggcctaccgaaagatcattattcgtcctcagaatgttagctgggaagtcgttgcatatgatgatcccaaaattccacttttcaacacagatgtggacaacctagaagggaagacaccaccagtttttgcttctgaaggcaaatacagggctctgaaaatggatttttctctacccccttctacttacgccaccatggccattcgagaagtgctaaaaatggataccagtatcaagaaccagacgcagctgaatacaacctggcttcgctgagcagtaccttgtccacagattagaaaacgtacacaagtgtttgcttcctggctccctgtgcatttttgtcttagttcagactcatatatggatttcaaatctttgtaataaaaattatttgtatttttaagtttttattagcttaaagaaataatttgcaatatttgtacatgtacacaaatcctgaggttcttaattttagctcagaatataaattagtcaaaatacacttcaggtgcttaaatcagagtaaaatgtcagctttacaataataaaaaaaggactttggtttaaagtagcaggtttaggttttgctacattctcaaaagacagcaggagtatttgacacatctgtgatggagtatacaacaatgcattttaagagcaaatgcaacaaaacaaatctggactatggataaataatttgagagctgccacccacaaatataaatacagtactcatgctgactgaaataataagacatctacaaatttataaacaaaaagtgattgtcattatcctgcttatgtactagattcaggcaagcattatagactttttggttgcggtggcttttgcatttatattatcaatgccttgcaggaacgttgcattgataggcccattttatttttttattttttttttcgagacaggatctcactctgtagcacaggctggattgcagtgcaatcctgcaattctcaatcttgcactgcagcctcgacctcccaggctccagtgactctcccacctcagcctcctaagtagctgggagtacaggcgcgcaccaccacgcctagctgatttttgtatttttttgtagagacgggggtttggccatgttgccgaggctaactcctgggattacaggcatgagctgtgctggccgggtttttttttcttgatgtaaacgtgtacagctgttttattagttaaggtctaatttttactctaggtgccttttatgttcagaactctttccactggactggtatttgctcaaaaataaataatggtagagaagaaaactataaaaatggacaaggctttcttctatcagtagcgtttaccctttgtcaccagtggctttggtatttccatgtctggcattgcataaacttctctggtgtgaaaggataaatatgcctttctaaagttgtatatcaaaattgtatcaatttttattttctatgatttctagaaacaaatgtaataaatatttttaaaatctcctttctactggttatgtaaataaatcaaataaatatatc

SEQ ID NO: 14 - protein, 795 aa, Homo sapiens [sequence omitted]

SEQ ID NO: 15 - DNA, 2335 bp, Homo sapiens
gggcaccagttgtcaggagttgacaggagggcatagccaggaacagctggtgtctccttactgggacatgctgtgacctgtagaatcgtgtctgaccatttctggaggatgcagtactgaattcaagtaacaaggtgtcctgtgaaccccccacagtcagatgggtgagggatggggagggtcacgcagggtgtgggcggcgtgcctggcatgggtcctgccaagttcgtcaggtaacgtgatacttctcccttgctttttggtagatggacagtgtgtcaccctcggggagcaggtgggctggcaggacaggcggccccaggccgggagaaggagacactcccaggtcggtaggctccacgacaaaagtcaacccttctgtaaatcacctgctgtggttatgatgctctgagttcaatacgtctgaacctttgctgtctatggatctgctctaaaccttatagcctgcttatgggggaaggtgacgccttctgggccccatctgtccttcctcacagcaccctcagcaccttaagccaccaccctcagccacaatttggcagaaggatggagtccaaggtctcagaaggtggcctgaatgtgaccctcaccatccgcctgctgatgcatggaaaggaagttggaagcatcatcgggaagaaaggagaaactgtgaagaagatgcgtgaggagagtggtgcaaggatcaacatctcagagggaaactgcccagagaggattgtgaccatcacaggccccacagacgccatcttcaaggcctttgccatgatcgcatacaagtttgaggaggatatcatcaactccatgagcaacagccctgccaccagcaagcccccagtgacgctgaggctggtggtgcctgccagccagtgcgggtccctgatcggcaaaggaggctccaagatcaaggagatcagggagtccacaggtgcccaggtgcaggtggctggggacatgctgcccaactccacggagcgagcggtgaccatctcggggaccccagatgccatcatccagtgcgtcaagcagatctgtgtggtcatgctggagtccccaccgaaaggtgccaccattccctaccgcccaaagcccgcctccacccctgtcatttttgcaggtggtcaggcctacacaatccagggacagtatgccatccctcacccggatcagttgaccaagctccaccagttggccatgcagcaaaccccctttcctcccctcggacagaccaaccccgctttccccggagaaaagctgcctttacactcctccgaagaagctcaaaatctgatgggccagtcatcaggtctggacgccagcccaccggccagcactcatgagctcaccattcccaatgatctaataggctgcataattggacgccaagggaccaaaatcaatgaaattcgacagatgtctggagctcagatcaaaatcgccaacgccacggaagggtcctcagagcgtcagatcaccatcacggggaccccggccaacatcagccttgcccagtatctcatcaacgccaggctgacgtccgaggtcaccgggatgggcacgctgtaatcctacccagcacccttcccccgcgtcacccacctgccagagcctaaggcccccggctctcgcactctgtacagcccaccttccctgcctcacagataccaatagagaggttttcttaattaacaaaaggacgtatgccatggagaaacacacccgcgcacacagctgctctctacagaggctgcaggctccgccgagtcccccctcagtgttattttatttatgacttacgctcccgtctgcccatgcaccggcatgcagtggtaattattttagaaatattgttccttggtgtcagcgtagctgtctgtcttaggagctgggtcggcgttccgacagcacttcctgtccgcccttctcctctgccatccagaaccgtccagaactgttgcctgagacccctcctctctcacacagccctgccatgctgactcggtttcccctcagagccattgttgtctgggctcgagtttctgccccaggttgtgtgctggaatcggggggtggctctcctgccacccatggggagcgccaggagaggagggtcatggaggatgttggggctctgaccccaggagtggggtggagggcggagcctgctggaggccctgccttcacagagatgccgcgtgctgggaaggctcttggggtcccctgagcgtcttccagggtggctggagagcacagacgcgccagggagccccctctgtgctcctcagagttcaataaatgtcgtggcccctcctca

SEQ ID NO: 16 - protein, 371 aa, Homo sapiens
MGEGDAFWAPSVLPHSTLSTLSHHPQPQFGRRMESKVSEGGLNVTLTIRLLMHGKEVGSIIGKKGETVKKMREESGARINISEGNCPERIVTITGPTDAIFKAFAMIAYKFEEDIINSMSNSPATSKPPVTLRLVVPASQCGSLIGKGGSKIKEIRESTGAQVQVAGDMLPNSTERAVTISGTPDAIIQCVKQICVVMLESPPKGATIPYRPKPASTPVIFAGGQAYTIQGQYAIPHPDQLTKLHQLAMQQTPFPPLGQTNPAFPGEKLPLHSSEEAQNLMGQSSGLDASPPASTHELTIPNDLIGCIIGRQGTKINEIRQMSGAQIKIANATEGSSERQITITGTPANISLAQYLINARLTSEVTGMGTL

SEQ ID NO: 17 - DNA, 5184 bp, Homo sapiens
ggatccggattcggattagcagcccgggaagagtgccgtggcacaggcgccggagggagcgcgaccctcggaccccgcctggcccgcggggctgggacccggccccggcctgcccgatggggcgcgcggccccggagatgcgccctcgcccggccccgcgcccccggccccgcgcccccggcccgcccgccccggcccgcgcctccgcctgagtcccccgcgccttggcccgccaccccccgccccgcgcccccggcccgcctgcgccatggagcccggccgcggcggcacagagaccgtgggcaagttcgagttctcccgcaaggacctgatcggccacggcgccttcgcggtggtcttcaagggccgccaccgcgagaagcacgatttggaggtcgccgtcaagtgcattaacaagaagaacctcgccaagtctcagacgctgctggggaaggaaatcaaaatcctgaaggaactgaaacatgaaaacatcgtggccctgtacgacttccaggaaatggctaattctgtctacctggttatggagtactgcaacggtggggacctggccgactacctgcacgccatgcgcacgctgagcgaggacaccatcaggctcttcctgcagcagatcgcgggcgccatgcggcttctgcacagcaaaggcatcatccaccgcgacctgaaaccgcagaacatcctgctgtccaaccccgccggccgccgcgccaaccccaacagcatccgcgtcaagatcgctgacttcggcttcgcgcggtacctccagagcaacatgatggcggccacactctgcggctcccccatgtacatggcccccgaggtcatcatgtcccagcactacgacgggaaggcggacctgtggagcatcggcaccatcgtctaccagtgcctgacggggaaggcgcccttccaggccagcagcccccaggacctgcgcctgttctacgagaagaacaagacgttggtccccaccatcccccgggagacctcggccccgctgcggcagctgctcctggccctactgcaacgcaaccacaaggaccgcatggacttcgatgagttttttcatcaccctttcctcgatgccagcccctcggtcaggaaatccccacccgtgcctgtgccctcgtacccaagctcggggtccggcagcagctccagcagcagctccacctcccacctggcctccccgccgtccctgggcgagatgcagcagctgcagaagaccctggcctccccggctgacaccgctggcttcctgcacagctcccgggactctggtggcagcaaggactcttcctgtgacacagatgacttcgtcatggtccccgcgcagtttccaggtgacctggtggctgaggcgcccagtgccaaacccccgccagacagcctgatgtgcagtgggagctcactggtggcctctgcgggcttggagagccacggccggaccccatctccatccccaccctgcagcagctcccccagtccctcaggccgggctggcccgttctccagcagcaggtgcggcgcctctgtccccatcccagtccccacgcaggtgcagaactaccagcgcattgagcgaaacctgcagtcacccacccagttccaaacacctcggtcctctgccatccgcaggtcaggcagcaccagccccctgggctttgcaagggccagcccctcgccccctgcccacgctgagcatggaggcgtcctggccaggaagatgtctctgggtggaggccggccctacacgccatctcctcaagttggaaccatccctgagcggccaggctggagcgggacgccctccccacagggagctgagatgcggggtggcaggtcccctcgtccaggctcctctgcacccgagcactctccccgcacttccgggctgggctgccgcctgcacagcgcccccaacctgtctgacttgcacgtcgtccgccccaagctgcccaaaccccccacggaccccctgggagctgtgttcagcccaccacaggccagccctccccagccgtcccacggcctgcagtcctgccggaacctgcggggctcacccaagctgcccgacttcctgcagcgaaaccccctgccccccatcctgggctcccccaccaaggctgtgccctcctttgacttcccgaagacccccagctcccagaacctgctggccctcctagcccggcagggcgtggtgatgacgccccctcgaaaccggacgctgcccgacctctcggaggtgggacccttccatggtcagccgttgggccctggcctgcggccaggcgaggaccccaagggcccctttggccggtctttcagcaccagccgcctcactgacctgctccttaaggcggcgtttgggacacaagccccggacccgggcagcacggagagcctgcaggagaagcccatggagatcgcaccctcagctggctttggagggagcctgcacccaggagcccgtgctgggggcaccagcagcccttccccggtggtcttcaccgtgggctctcccccgagcgggagcacgcccccccagggcccccgcaccaggatgttctcagcgggccccactggctctgccagctcttctgcccgccacctggtgcctgggccctgcagcgaggccccagcccctgagctccctgctccaggacacggctgcagctttgccgaccccattgctgcgaacctggagggggctgtgaccttcgaggcccccgacctccctgaggagaccctcatggagcaagagcacacggagatcctgcgtggcctgcgcttcacgctgctgttcgtgcagcacgtcctggagatcgcagccctgaagggcagcgccagtgaggcggcggggggccctgagtaccagctgcaggagagtgtggtggccgaccagatcagcctgctgagccgagaatggggcttcgcggaacagctggtgctgtacctgaaggtggccgagctactgtcctccggcctgcaaagtgccatcgaccagatccgggccggcaagctctgcctgtcgtccactgtgaagcaggtggtgcgcaggctgaatgagctgtacaaggccagcgtggtgtcctgccagggcctgagcctgcggctgcagcgcttcttcctggacaagcagcggctcctggaccgcattcacagcatcactgccgagaggctcatcttcagccacgctgtgcagatggtgcagtcggctgccctggacgagatgttccagcaccgtgagggctgcgtcccacgctaccacaaggccctgctgctcctggaggggctgcagcacatgctctcggaccaggccgacatcgagaacgtcaccaagtgcaagctgtgcattgagcggagactctcggcgctgctgactggcatctgtgcctgacctttctggcctggctgggccccccgtcctgccgagccctgcagagtgggctctgtgtgctggctggactcctcgggacaagcccatggcgctgatcgctggtgctgagccctgccctgggccccacggacagtcagcctgccggcctccctgcagctcacggggcagaaccagcacatctggagccacacagcttggggggtgtctcccatcttttacaggtggggatcacagaatttctgcccctccagctgcctggctcagcaggcgtgggtgccaccaccctctagccccagggcagccccggaggacaggcaagggcctgagaccactgccgactcaaagccaaagcgagctcctgcttagggcaggtcagcaggcactgtgcccaggaagagcctgcggcctcggcgtcccccagtctccaggagcctctccctccgagatacccacccagctttgtcaatcacccaagcactttatgcatatagagacagaacctggacctcaccagggactgctgggcagcgattcctggcagtggcctggtgtttgtacatacacatatgcagacacatgccagggccccccaagcccgagcaccggaccacgttgctgcccaggtctggacctcagcgggagaactggctccggggggagtggggccctgcgctagaggcagaggcagttctttgttcaagcgttcctctggggaccggcagcagaggcaccgtgttctctcagccctggatacgtcttgtaatctttcacactttattcctaaaacgtgtcttatttttatgcagctcattttttctttaaaggagaaaacttgtaggtgtttaagaattggttttgggagggcgaggactgggccaggttagaggcagatggcacaggggcgtgtggcgggcgggtgaggctgctttgcacacctgtgttggtggctgtcccctgccgcccctccctgtggcagcagcaggacaggtgtgtgcccagcaccctccctacctgggcctggaagcagatgaggggaatacttcatgcaaagaaaaaagtaacatgtgcaaaagctccccgtccagctttgacagtcagttttgatgtcagctcctcggcagggtaggcctgatgacagccctgtccctccctgcctctgccttgcccaaggccacggagggcgtctgcagagaggcctgcctcccttggtctgcccagccctcggttagccctgcctgaatcagtagatacttgaacgagtccccagtctgcgggaggcagtggtggggccatggacccatgcggggggttccagggtcacacgccacataacagacaaaaatacacacacgtgtgtttttctttgcaatacttgaaatattgccactgtgcttggacttagaagaagaaaatccccgtgacttcttcctcatcaccttgatggctttattctcaccttgtggggcatgtttgtatttattgcttcatggccgactggaatcctgagtcctgggaagctggcactgcggggatcttgcccggtgtcctggtcctcttgcttccgtcgcggccgcatgtgcgtgtgtccaagcaggtcctgggcgcctcaactgctgcccctggttgaatgttctcttgatagtgctggaccctttgtctattttaaagcgaattttgtgtgatttcctgccctttgcgttatattgtataataccaacgtaaggaaataaacctttggaattgttgggctggtgtcaccactt

SEQ ID NO: 18 - protein, 1050 aa, Homo sapiens
MEPGRGGTETVGKFEFSRKDLIGHGAFAVVFKGRHREKHDLEVAVKCINKKNLAKSQTLLGKEIKILKELKHENIVALYDFQEMANSVYLVMEYCNGGDLADYLHAMRTLSEDTIRLFLQQIAGAMRLLHSKGIIHRDLKPQNILLSNPAGRRANPNSIRVKIADFGFARYLQSNMMAATLCGSPMYMAPEVIMSQHYDGKADLWSIGTIVYQCLTGKAPFQASSPQDLRLFYEKNKTLVPTIPRETSAPLRQLLLALLQRNHKDRMDFDEFFHHPFLDASPSVRKSPPVPVPSYPSSGSGSSSSSSSTSHLASPPSLGEMQQLQKTLASPADTAGFLHSSRDSGGSKDSSCDTDDFVMVPAQFPGDLVAEAPSAKPPPDSLMCSGSSLVASAGLESHGRTPSPSPPCSSSPSPSGRAGPFSSSRCGASVPIPVPTQVQNYQRIERNLQSPTQFQTPRSSAIRRSGSTSPLGFARASPSPPAHAEHGGVLARKMSLGGGRPYTPSPQVGTIPERPGWSGTPSPQGAEMRGGRSPRPGSSAPEHSPRTSGLGCRLHSAPNLSDLHVVRPKLPKPPTDPLGAVFSPPQASPPQPSHGLQSCRNLRGSPKLPDFLQRNPLPPILGSPTKAVPSFDFPKTPSSQNLLALLARQGVVMTPPRNRTLPDLSEVGPFHGQPLGPGLRPGEDPKGPFGRSFSTSRLTDLLLKAAFGTQAPDPGSTESLQEKPMEIAPSAGFGGSLHPGARAGGTSSPSPVVFTVGSPPSGSTPPQGPRTRMFSAGPTGSASSSARHLVPGPCSEAPAPELPAPGHGCSFADPIAANLEGAVTFEAPDLPEETLMEQEHTEILRGLRFTLLFVQHVLEIAALKGSASEAAGGPEYQLQESVVADQISLLSREWGFAEQLVLYLKVAELLSSGLQSAIDQIRAGKLCLSSTVKQVVRRLNELYKASVVSCQGLSLRLQRFFLDKQRLLDRIHSITAERLIFSHAVQMVQSAALDEMFQHREGCVPRYHKALLLLEGLQHMLSDQADIENVTKCKLCIERRLSALLTGICA

SEQ ID NO: 19 - DNA, 4154 bp, Homo sapiens
cacagcggcggccgggtggcgggggtgagtggggccagcggggctggacagcagcgggccccgggcgccgccgccgcgatccctccccgcgcccgccgagcacatcgccgccgccgagatgggccctccgcggcacccccaggccggcgagatagaagcgggcggtgcgggcggcgggcggcggctacaggtggaaatgagttctcaacagtttcctcggttaggagccccttctaccgggctgagccaggccccttctcagattgcaaacagtggttctgctggattgataaacccagctgctacagtcaatgatgaatctggtcgagattctgaagtcagtgccagggagcacatgagttccagcagctccctccagtcccgggaggagaagcaagagcctgttgtggtaaggccctatccacaggtgcagatgttgtcgacacaccatgctgtcgcatcagccacacctgttgcagtgacagccccgccagcacacctgacgccagcagtgccactttcattttcggagggacttatgaagccgcccccgaagcccaccatgcctagccgtcccattgctcctgctccaccttctaccctgtcacttccccccaaggttccagggcaggttaccgttaccatggagagtagcatccctcaagcttcagccattcctgtggcaacaatcagtggacaacagggccatcccagtaacctgcatcacatcatgactacaaatgtgcaaatgtctatcatccgcagcaatgctcctgggccccctcttcacattggagcttctcatttacctcgaggtgcagctgctgctgctgtgatgtccagttctaaagtaaccacagtcctgaggccgacctcacagctgccaaatgctgctactgctcagccagcagtacagcacatcattcaccaaccaatccagtctcggccacctgtgaccacctccaatgccatccctcctgctgtggtagcaactgtctcagccaccagagctcagtctccagtcatcactacgacagcggcgcatgctactgattcagcacttagtaggccaaccttgtctatccagcatcctccatctgcagcaatcagtattcagcgtcctgcccagtcacgagatgtcacaacaagaatcacactaccatctcaccctgcattagggacgccaaaacagcagcttcatacaatggctcagaaaacaatcttcagtactggcacgccagtggctgcagccacagtagcacctattttggcaaccaacaccattccttcagcgaccacagctggatctgtgtcacacacgcaagctcccacaagtaccattgttaccatgacagtaccctcccattcctcccatgctactgctgtgaccacctcaaacatcccagtcgccaaggtggtgccccagcagatcacgcacacttctcctcggatccagccagactaccctgccgagaggagtagcctgattcccatctccggacatcgggcctctcccaatcctgtggccatggaaacccgaagtgacaacagaccgtctgttcccgttcagttccaatattttttgccaacttaccccccttctgcatacccactggcggcacatacctacaccccaatcaccagttccgtgtccactatccgacagtatccagtttcagctcaggctccaaactctgccatcacagctcagactggtgttggggtagcgtctaccgtccacctaaaccccatgcagttgatgacagtggatgcatcgcatgctcgacatattcaagggatccagccagcacccatcagtacccagggtatccagccggcccccattgggaccccagggatacagcctgcaccacttggcacacagggaattcactcagcaaccccaatcaacacacaagggcttcagcctgcacctatgggtactcagcagcctcagcctgaaggaaagacttcagcagtggtgttggcagatggagccacaattgtggccaaccctattagcaatccattcagtgctgctccagcagcaacaaccgtggtgcagacccacagccagagtgctagcaccaacgctcccgcccagggctcatcgccacggccaagcatactccggaagaaacctgccacagatggaatggcagttcggaaaaccctcattcctcctcagcctcctgatgttgctagtcctcgagtggaaagctctatgcggagtacgtctgggtcacctaggcctgcaggtgccaaacccaagtctgaaatccacgtgtctatggccactccggtcactgtgtccatggagactgtatccaatcaaaataatgatcagcctaccattgccgtccctccaactgcccagcagcccccaccgaccattccaactatgattgcagcagccagtcccccgtcacaaccagccgttgccctttcaaccattcctggagcggtccccatcactccacccatcaccaccattgcagctgcaccacctccatcagtcactgtgggtggcagtctttcctccgtcttgggccctcccgttcctgaaattaaagtgaaagaagaagtagaaccaatggatatcatgaggccagtttctgcagttcctccactggctaccaacactgtgtctccatctcttgcattgctggcaaacaacttgtccatgcctacaagtgacctaccacctggtgcctccccaaggaaaaagcctcgaaagcaacagcatgtgatctcaacagaagaaggtgacatgatggagacaaacagcactgatgatgagaagtccactgccaagagtcttctggtgaaggctgagaagcgcaagtctcctcccaaggagtatattgatgaggaaggtgtgagatatgtcccagtgcgtccaagaccccccattactttgcttcgtcactatcggaacccctggaaagctgcttaccaccactttcagaggtacagtgacgtccgggtcaaagaggagaagaaagctatgctgcaggaaatagctaatcagaaaggagtatcctgtcgtgctcaaggctggaaagtccacctctgtgctgcccagttactacagctgacgaatctagaacatgatgtctatgaaagacttactaacctgcaggaagggattatcccaaagaaaaaagcagcaacagatgatgatctccaccgaataaacgaactgatacagggaaatatgcagaggtgtaaacttgtgatggatcaaatcagtgaagccagagactccatgcttaaggttttagatcataaagaccgtgtcctgaagctgcttaacaagaacgggactgtcaaaaaagtgtccaaattgaagcgaaaggaaaaagtctagacccagaacaatcaggagattggaagcaaatttatgaagaatgatggtgggggtggggggagggttttggttttttccaaagtggaacattgaaataaaggaagtgttccttagttcccgtgtgaaagcagaggaacccatgacatccaagggcgtgaaaggatcagagctgactggacatagtgagctgccttcttgcgttcgggtgcacccctgttaaacctgatctgtgtcataagtgactccggatgcatcagtgtccaccagttggaagcaatgacaaggatggctggctggtgtttttcagccttccggtttatagactgtatttatctagtggattcctgcaggccccatactgagcctggactgaaagtatccactcggaccatctgttatctctctacactgaaaataaaacctcttccacccaccccattcggttcttctgcctgaccttcaaatgcccatgttggccttttacagcagtgccacggcaccaagcgagctgccacatctcacactctaaagggtttgaactattagttcttgtcattttttaaaaaaaaccattcccaagtgaaattgttatatcgtctgtcttgcgtgtgtcagaactgggtttttgtggaggttcagagcaggcaacaccataagttgctctcagatccttgttctgaagtacattcttggttatctgtacttctgtagctggtgtgatgctgttaattgtatgtaccacacatctccagacgttaataaaggactcaaagaggtttttgta

SEQ ID NO: 20 - protein, 1083 aa, Homo sapiens
MGPPRHPQAGEIEAGGAGGGRRLQVEMSSQQFPRLGAPSTGLSQAPSQIANSGSAGLINPAATVNDESGRDSEVSAREHMSSSSSLQSREEKQEPVVVRPYPQVQMLSTHHAVASATPVAVTAPPAHLTPAVPLSFSEGLMKPPPKPTMPSRPIAPAPPSTLSLPPKVPGQVTVTMESSIPQASAIPVATISGQQGHPSNLHHIMTTNVQMSIIRSNAPGPPLHIGASHLPRGAAAAAVMSSSKVTTVLRPTSQLPNAATAQPAVQHIIHQPIQSRPPVTTSNAIPPAVVATVSATRAQSPVITTTAAHATDSALSRPTLSIQHPPSAAISIQRPAQSRDVTTRITLPSHPALGTPKQQLHTMAQKTIFSTGTPVAAATVAPILATNTIPSATTAGSVSHTQAPTSTIVTMTVPSHSSHATAVTTSNIPVAKVVPQQITHTSPRIQPDYPAERSSLIPISGHRASPNPVAMETRSDNRPSVPVQFQYFLPTYPPSAYPLAAHTYTPITSSVSTIRQYPVSAQAPNSAITAQTGVGVASTVHLNPMQLMTVDASHARHIQGIQPAPISTQGIQPAPIGTPGIQPAPLGTQGIHSATPINTQGLQPAPMGTQQPQPEGKTSAVVLADGATIVANPISNPFSAAPAATTVVQTHSQSASTNAPAQGSSPRPSILRKKPATDGMAVRKTLIPPQPPDVASPRVESSMRSTSGSPRPAGAKPKSEIHVSMATPVTVSMETVSNQNNDQPTIAVPPTAQQPPPTIPTMIAAASPPSQPAVALSTIPGAVPITPPITTIAAAPPPSVTVGGSLSSVLGPPVPEIKVKEEVEPMDIMRPVSAVPPLATNTVSPSLALLANNLSMPTSDLPPGASPRKKPRKQQHVISTEEGDMMETNSTDDEKSTAKSLLVKAEKRKSPPKEYIDEEGVRYVPVRPRPPITLLRHYRNPWKAAYHHFQRYSDVRVKEEKKAMLQEIANQKGVSCRAQGWKVHLCAAQLLQLTNLEHDVYERLTNLQEGIIPKKKAATDDDLHRINELIQGNMQRCKLVMDQISEARDSMLKVLDHKDRVLKLLNKNGTVKKVSKLKRKEKV

SEQ ID NO: 21 - DNA, 2460 bp, Homo sapiens
  misc_feature (13)..(13): n is a, c, g, or t
ggaaagttgtgantgtcgancttgatcctggcgngccgcggagttcaccctgnggcccnggagcaaagtactagctctgtgattagattgaattctccaacaacaacatctcagattatggcaagaaagaaaaggagagggattatagagaaaaggcgtcgggatcggataaataacagtttatctgagttgagaagacttgtgccaactgcttttgaaaaacaaggatctgcaaagttagaaaaagctgaaatattgcaaatgacagtggatcatttgaagatgcttcaggcaacagggggtaaaggctactttgacgcacacgctcttgccatggacttcatgagcataggattccgagagtgcctaacagaagttgcgcggtacctgagctccgtggaaggcctggactcctcggatccgctgcgggtgcggcttgtgtctcatctcagcacttgcgccacccagcgggaggcggcggccatgacatcctccatggcccaccaccatcatccgctccacccgcatcactgggccgccgccttccaccacctgcccgcagccctgctccagcccaacggcctccatgcctcagagtcaaccccttgtcgcctctccacaacttcagaagtgcctcctgcccacggctctgctctcctcacggccacgtttgcccatgcggattcagccctccgaatgccatccacgggcagcgtcgccccctgcgtgccacctctctccacctctctcttgtccctctctgccaccgtccacgccgcagccgcagcagccaccgcggctgcacacagcttccctctgtccttcgcgggggcattccccatgcttcccccaaacgcagcagcagcagtggccgcggccacagccatcagcccgcccttgtcagtatcagccacgtccagtcctcagcagaccagcagtggaacaaacaataaaccttaccgaccctgggggacagaagttggagctttttaaatttttcttgaacttcttgcaatagtaactgaatgtcctccatttcagagtcagcttaaaacctctgcaccctgaaggtagccatacagatgccgacagatccacaaaggaacaataaagctatttgagacacaaacctcacgagtggaaatgtggtattctcttttttttctctcccttttttgtttggttcaaggcagctcggtaactgacatcagcaacttttgaaaacttcacacttgttaccatttagaagtttcctggaaaatatatggaccgtaccatccagcagtgcatcagtatgtctgaattggggaagtaaaatgccctgactgaattctcttgagactagatgggacatacatatatagagagagagtgagagagtcgtgtttcgtaagtgcctgagcttaggaagttttcttctggatatataacattgcacaagggaagacgagtgtggaggataggttaagaaaggaaagggacagaagtcttgcaataggctgcagacattttaataccatgccagagaagagtattctgctgaaaccaacaggttttactggtcaaaatgactgctgaaaataattttcaagttgaaagatctagttttatcttagtttgccttctttgtacagacatgccaagaggtgacatttagcagtgcattggtataagcaattatttcatcagttctcagattaacaagcatttctgctctgcctgcaggcccccaggcacttttttttttggatggctcaaaatatggtgcttctttatataaaccttacatttatatagtgcacctatgagcagttgcctaccatgtgtccaccagaggctatttaattcatgccaacttgaaaactctccagtttgtaggagtttggtttaatttattcagtttcattaggactatttttatatatttatcctcttcattttctcctaatgatgcaacatctattcttgtcaccctttgggagaagttacatttctggaggtgatgaagcaaggagggagcactaggaagagaaaagctacaatttttaaagctctttgtcaagttagtgattgcatttgatcccaaaacaagatgaatgtatgcaatgggatgtacataagttatttttgcccatgcctaaactagtgctatgtaatggggttgtggttttgtttttttcgatttcgtttaatgacaaaataatctcttaatatgctgaaatcaagcacgtgagagtttttgtttaaaagataagagacacagcatgtattatgcacttcatttctctactgtgtggagaaagcaataaacattatgagaatgttaaacgttatgcaaaattatacttttaaatatttgttttgaaattactgtacctagtcttttttgcattactttgtaacctttttctatgcaagagtctttacataccactaattaaatgaagtcctttttgacta

SEQ ID NO: 22 - protein, 328 aa, Homo sapiens
  misc_feature (3)..(3): Xaa can be any naturally occurring amino acid
VVXVXLILAXRGVHPXAXEQSTSSVIRLNSPTTTSQIMARKKRRGIIEKRRRDRINNSLSELRRLVPTAFEKQGSAKLEKAEILQMTVDHLKMLQATGGKGYFDAHALAMDFMSIGFRECLTEVARYLSSVEGLDSSDPLRVRLVSHLSTCATQREAAAMTSSMAHHHHPLHPHHWAAAFHHLPAALLQPNGLHASESTPCRLSTTSEVPPAHGSALLTATFAHADSALRMPSTGSVAPCVPPLSTSLLSLSATVHAAAAAATAAAHSFPLSFAGAFPMLPPNAAAAVAAATAISPPLSVSATSSPQQTSSGTNNKPYRPWGTEVGAF

SEQ ID NO: 23 - DNA, 1935 bp, Homo sapiens
agctaatcgttgccagcggggtgtggacttcgccgctgaccccacctccgccgctttgggtaatttagagccgcgcgccgggcgggaatgtaagatggcggagtagcaacgcaaagcgcttggtattgagtctgtggccgacttcggttccggtctctgcagcagccgtgatcgcttagtggagtgcttagggtagttggccaggatgccgaatatcaaaatcttcagcggcagctcccaccaggacttatctcagaaaattgctgaccgcctgggcctggagctaggcaaggtggtgactaagaaattcagcaaccaggagacctgtgtggaaattggtgaaagtgtacgtggagaggatgtctacattgttcagagtggttgtggcgaaatcaatgacaatttaatggagcttttgatcatgattaatgcctgcaagattgcttcagccagccgggttactgcagtcatcccatgcttcccttatgcccggcaggataagaaagataagagccgggcgccaatctcagccaagcttgttgcaaatatgctatctgtagcaggtgcagatcatattatcaccatggacctacatgcttctcaaattcagggcttttttgatatcccagtagacaatttgtatgcagagccggctgtcctaaagtggataagggagaatatctctgagtggaggaactgcactattgtctcacctgatgctggtggagctaagagagtgacctccattgcagacaggctgaatgtggactttgccttgattcacaaagaacggaagaaggccaatgaagtggaccgcatggtgcttgtgggagatgtgaaggatcgggtggccatccttgtggatgacatggctgacacttgtggcacaatctgccatgcagctgacaaacttctctcagctggcgccaccagagtttatgccatcttgactcatggaatcttctccggtcctgctatttctcgcatcaacaacgcatgctttgaggcagtagtagtcaccaataccatacctcaggaggacaagatgaagcattgctccaaaatacaggtgattgacatctctatgatccttgcagaagccatcaggagaactcacaatggagaatccgtttcttacctattcagccatgtccctttataatagagtaacttctgaggctttttgagaataaaatccaccccacccttgtttccccttggtatttgatgacaaattcagcagaagacccggcttgctccagtgtagctttctacatcccacatcaggtatattagagcttatccgaactggggaaagacggattgagattaactgctgggacctcctacctgcattatctcattctggcttccttgataattctgtgggccttgcagctttaactatagctcagctgctgcaagatttcagacttttgaggatgttgtggaagaggccagaaaatggaagagacgcccctgtaacgggaatgctaatgaggaacatggggagcaggaggctgacaatgaggtagacgacgcagaggaagaaggtgcggcggcaagacggacgcggaaggaaacgaacggtgcctggtgaccccacgaaggctggaccgatggacgatgacggaagcctgagtctagctaccgggcaagccggcgcctgcagaatgattgaggctcacgacgtcgctccagaagctgagaccgacagcgctgctcaccgccaacacggctctgttctcttaacaacaaggcgcgtgactcatagcctcatccgtctagcactacggtccttgagtacaggccccgccacggactgccccgtagcggtctcccacctcccacctgcgtggaacacaccgggtcacagcaaacgcgcgccctaccgtttgtcaacgaaccgccctattgtacccacccccccttttt

SEQ ID NO: 24 - protein, 317 aa, Homo sapiens
PNIKIFSGSSHQDLSQKIADRLGLELGKVVTKKFSNQETCVEIGESVRGEDVYIVQSGCGEINDNLMELLIMINACKIASASRVTAVIPCFPYARQDKKDKSRAPISAKLVANMLSVAGADHIITMDLHASQIQGFFDIPVDNLYAEPAVLKWIRENISEWRNCTIVSPDAGGAKRVTSIADRLNVDFALIHKERKKANEVDRMVLVGDVKDRVAILVDDMADTCGTICHAADKLLSAGATRVYAILTHGIFSGPAISRINNACFEAVVVTNTIPQEDKMKHCSKIQVIDISMILAEAIRRTHNGESVSYLFSHVPL

SEQ ID NO: 25 - DNA, 1054 bp, Homo sapiens
atgcgccgacccggcgccattttggtggccgggcgcggaggtgattccacactgaggcgagcgcggcggccggggtggtagtggcagtgttcgtgtgctcaggtctgaatcgccgagggaggaggcggtggaggaagaggtggcggcggtggcggtggtcgtagcggtggcggaggaggcgggtacgaatcagctgcgggcggagacatggccaacatcgcggtgcagcgaatcaagcgggagttcaaggaggtgctgaagagcgaggagacgagcaaaaatcaaattaaagtagatcttgtagatgagaattttacagaattaagaggagaaatagcaggacctccagacacaccatatgaaggaggaagataccaactagagataaaaataccagaaacatacccatttaatccccctaaggtccggtttatcactaaaatatggcatcctaatattagttccgtcacaggggctatttgtttggatatcctgaaagatcaatgggcagctgcaatgactctccgcacggtattattgtcattgcaagcactattggcagctgcagagccagatgatccacaggatgctgtagtagcaaatcagtacaaacaaaatcccgaaatgttcaaacagacagctcgactttgggcacatgtgtatgctggagcaccagtttctagtccagaatacaccaaaaaaatagaaaacctatgtgctatgggctttgataggaatgcagtaatagtggccttgtcttcaaaatcatgggatgtagagactgcaacagaattgcttctgagtaactgaggcatagagagctgctgatatagtcaagcttgcctcttcttgaggagcaccaacatctgttatttttaggattctgcatagatttcttttaaactggcattcttgcctaatgatgttatctaggcaccattggagactgaactatctaaagagtgacagccccgcgatacacccgggagcattgtgtgactcacgaacaacgtcccgtgtctatgggggccctggtgtaacattgatcggttgt

SEQ ID NO: 26 - protein, 200 aa, Homo sapiens
MANIAVQRIKREFKEVLKSEETSKNQIKVDLVDENFTELRGEIAGPPDTPYEGGRYQLEIKIPETYPFNPPKVRFITKIWHPNISSVTGAICLDILKDQWAAAMTLRTVLLSLQALLAAAEPDDPQDAVVANQYKQNPEMFKQTARLWAHVYAGAPVSSPEYTKKIENLCAMGFDRNAVIVALSSKSWDVETATELLLSN

SEQ ID NO: 27 - DNA, 5719 bp, Homo sapiens
tgcgcagggccggctcgaagcgcaagcaggaagcgtttgcggtgatcccggcgactgcgctggctaatgcggtaccggctagcgtggcttctgcaccccgcactgcccagcaccttccgctcagtcctcggcgcccgcctgccgcctccggagcgcctgtgtgggaactaccaggttgtttttcaaagcagcggcactattttgcatttccaccagcagtgtgtgaaaattccaatttctctatatccttgacaacacttgctatatgtacttttgttataagccatcctggtgggtatgaagtggtgtctcattggaattttgactttcattttcctgatgaccattgtaagagttttaaaatatattctctaggccgggcgtgttggctcatgcctgtaatcccagcactttgggaggatgaggcaggtggatcacctgcagtcaggagttcaggaccagcctgaccaacaagctcctgcatgtggcatctgaaagctatctgtgaagaggttcaagcaattcttctgcctcagcctcccgagtagctgggattacagtttccaaaaaaagacttacagcaaaatgaataatccagccatcaagagaataggaaatcacattaccaagtctcctgaagacaagcgagaatatcgagggctagagctggccaatggtatcaaagtacttcttatcagtgatcccaccacggataagtcatcagcagcacttgatgtgcacataggttcattgtcggatcctccaaatattgctggcttaagtcatttttgtgaacatatgctttttttgggaacaaagaaataccctaaagaaaatgaatacagccagtttctcagtgagcatgcaggaagttcaaatgcctttactagtggagagcataccaattactattttgatgtttctcatgaacacctagaaggtgccctagacaggtttgcacagttttttctgtgccccttgttcgatgaaagttgcaaagacagagaggtgaatgcagttgattcagaacatgagaagaatgtgatgaatgatgcctggagactctttcaattggaaaaagctacagggaatcctaaacaccccttcagtaaatttgggacaggtaacaaatatactctggagactagaccaaaccaagaaggcattgatgtaagacaagagctactgaaattccattctgcttactattcatccaacttaatggctgtttgtgttttaggtcgagaatctttagatgacttgactaatctggtggtaaagttattttctgaagtagagaacaaaaatgttccattgccagaatttcctgaacaccctttccaagaagaacatcttaaacaactttacaaaatagtacccattaaagatattaggaatctctatgtgacatttcccatacctgaccttcagaaatactacaaatcaaatcctggtcattatcttggtcatctcattgggcatgaaggtcctggaagtctgttatcagaacttaagtcaaagggctgggttaatactcttgttggtgggcagaaggaaggagcccgaggttttatgttttttatcattaatgtggacttgaccgaggaaggattattacatgttgaagatataattttgcacatgtttcaatacattcagaagttacgtgcagaaggacctcaagaatgggttttccaagagtgcaaggacttgaatgctgttgcttttaggtttaaagacaaagagaggccacggggctatacatctaagattgcaggaatattgcattattatcccctagaagaggtgctcacagcggaatatttactggaagaatttagacctgacttaatagagatggttctcgataaactcagaccagaaaatgtccgggttgccatagtttctaaatcttttgaaggaaaaactgatcgcacagaagagtggtatggaacccagtacaaacaagaagctataccggatgaagtcatcaagaaatggcaaaatgctgacctgaatgggaaatttaaacttcctacaaagaatgaatttattcctacgaattttgagattttaccgttagaaaaagaggcgacaccataccctgctcttattaaggatacagctatgagcaaactttggttcaaacaagatgataagttttttttgccgaaggcttgtctcaactttgaatttttcagcccatttgcttatgtggaccccttgcactgtaacatggcctatttgtaccttgagctcctcaaagactcactcaacgagtatgcatatgcagcagagctagcaggcttgagctatgatctccaaaataccatctatgggatgtatctttcagtgaaaggttacaatgacaagcagccaattttactaaagaagattattgagaaaatggctacctttgagattgatgaaaaaagatttgaaattatcaaagaagcatatatgcgatctcttaacaatttccgggctgaacagcctcaccagcatgccatgtactacctccgcttgctgatgactgaagtggcctggactaaagatgagttaaaagaagctctggatgatgtaacccttcctcgccttaaggccttcatacctcagctcctgtcacggctgcacattgaagcccttctccatggaaacataacaaagcaggctgcattaggaattatgcagatggttgaagacaccctcattgaacatgctcataccaaacctctccttccaagtcagctggttcggtatagagaagttcagctccctgacagaggatggtttgtttatcagcagagaaatgaagttcacaataactgtggcatcgagatatactaccaaacagacatgcaaagcacctcagagaatatgtttctggagctcttctgtcagattatctcggaaccttgcttcaacaccctgcgcaccaaggagcagttgggctatatcgtcttcagcgggccacgtcgagctaatggcatacagggcttgagattcatcatccagtcagaaaagccacctcactacctagaaagcagagtggaagctttcttaattaccatggaaaagtccatagaggacatgacagaagaggccttccaaaaacacattcaggcattagcaattcgtcgactagacaaaccaaagaagctatctgctgagtgtgctaaatactggggagaaatcatctcccagcaatataattttgacagagataacactgaggttgcatatttaaagacacttaccaaggaagatatcatcaaattctacaaggaaatgttggcagtagatgctccaaggagacataaggtatccgtccatgttcttgccagggaaatggattcttgtcctgttgttggagagttcccatgtcaaaatgacataaatttgtcacaagcaccagccttgccacaacctgaagtgattcagaacatgaccgaattcaagcgtggtctgccactgtttccccttgtgaaaccacatattaacttcatggctgcaaaactctgaagattccccatgcatgggaaagtgcaagtggatgcattcctgagtcttccagagcctaagaaaatcatcttggccactttaatagtttctgattcactattagagaaacaaacaaaaaattgtcaaatgtcattatgtagaaatattataaatccaaagtaaattacaaaatcttatagatgtagaatattttttaaatacatgcctcttaaatattttaaaatttttcttttgattactgagagaaatttccccaatataacaatgcttaaaatgaatgatattcctatagaatcttccttccctattctgtaaaatagtcacttgtccgaagaaagttaaaagttagctcttttctaaaagcctcctagcttgacatagaaggcttcacaacatttagaaaggtaataactttttaaaaattgatcctcaaatttgctttctacttgatggtttcatgtaaatcagtggaaaacattacatttggcagatgataaagcaatgtcatcttttattagtgaaatgctggttatataaggcatggttttaatctttttataaaatttgaacatgttttttatgccaactcgtaaaatgctagaaaaccctacttatttacaatgctagaaatacagacttaccttacatcaattttgtcctaaaccgaatttctcaggattactgtggtttctttcattctgattgaattatattgacctacttcttcatagttggtttgcagtgttccatgagttttacttttcctcatcaacatattgctttaacacaacatatttatttaacacgtacaaatagggtcaacttcagatcctactgagtgtgtgacatgcttttccaacatcagctttttgtaaccacctgtataactttttattacagtgaaattgcagtcagtatgtgaaccaaaatatcttgcccctttatgaatttaaaaggcagccaatacaaagccacctttttggaaatataaaaagtaaagccttgcattcttatatagcaggtcttcataaaactctaaaatcccttgttgctaccagtctaatcttgccttaaatgttaagttattttttgaatatataaatataaacatataaacacagatgatgactggagtagacttttaaaaaaatatttttttcatgagatactattttaggtgaaattgttactgtagatttaacagctgttttgaaatatttactgttattaaaacttgcttcaagagaaattgtgaatatatttccatatacaagcactagtaacagtaagtggccctgtcatccactaactcaggcaaagtaaagaatggcatttttgaaggacattttacctccccatatgatttgattggctaggactttcttctgtaaagtcataccttttcacatcttaagtttttacatttgccattttccaaatctcaattttgggcaagaacgatatagtcacaactatggggctgctttcaaaagcggggctccatttctactgtcagatcaatgtggtgctgtaaccatctttttatccctaccttcaagaacctccttatatgaagcctgtctttatccatcagaaggtgtgtgaaatcatcacttccttctggttttatgtatttgtagactatgcagcttttcattaaactgcaagtatatacaagacagatctgaaattaggcctgagtgttccgatccaccactgtactagtaaataaaaatccacctaccttttatgtggaaaattatgtgctattgagtaacttttagctcttttttaaaaaatgggtgaaatttaagtgtcttttttatgagaatgacacatgaagagatctgagagcaatctcatgtagtcttccatgaacctgcaattgtttggtatgcgtcagcattttccaatttccaggttggatctagagctgctgttgatcactcaggcatactaatggattcatttagatgggtccaagctgcagtccatgagcaataacagactaccccagatactgcagtttacgcagtgcttagtaaatgagatttgtggaactaagttattagttacctgaggcttcttaagaaagtcttcttttttgaccagttgatgtgaaagagggagcatgtgacacagccagtatggtggagtgctagggttatcctgtttacaataaatcgcctgaatttca

SEQ ID NO: 28 - protein, 1173 aa, Homo sapiens [sequence omitted]

SEQ ID NO: 29 - DNA, 4768 bp, Homo sapiens
gcacgaggggaaagttgaaaagaactttgaagagagagacaaggagaagaagaaggagaaggagaagaaggctgaggacctggacttctggctgtctaccaccccaccgcctgcccccgcccccgcccccgcccccgttccatccacgggggagctcagtgtgaacactgtcactaccccgaaggacgagtgtgaggacgccaagacggaggcgcagggcgaggaggacgatgccgaggggcaagaccaggacaagaaatctcccaagcctaagaagaagaagcacaggaaggagaaggaggagcggaccaaaggcaagaagaagtccaagaagcagcctccaggcaggaggaggagcagctcccgcctgagtccagctactccctcctcgctgaaaattcctatgttaaaatgacctgtgacatccggggcagtctgcaggaggacagccaggtcactgtggccatcgtgctggagaacaggagcagcagcatcctcaagggcatggagctcagcgtgctggactcactcaatgccaggatggcccggccgcagggctcctccgtccacgatggcgtccccgtgcctttccagctgcccccaggcgtctccaacgaagcccagtatgtgttcaccatccagagcatcgtcatggcgcagaagctcaaggggaccctgtccttcattgccaagaatgacgagggtgcgacccacgagaagctggacttcaggctgcacttcagctgcagctcctacttgatcaccactccctgctacagtgacgcctttgctaagttgctggagtctggggacttgagcatgagctcaatcaaagtcgatggcattcggatgtccttccagaatcttctggcgaagatctgttttcaccaccatttttccgttgtggagcgagtggactcctgcgcctccatgtacagccgctccatccagggccaccatgtctgcctcctggtgaaaaagggtgagaactctgtctcagtcgacgggaagtgcagtgactccacgctactgagcaacttgttagaagagatgaaggcgacgctggccaagtgttgagagctgcctgcgagccccgcaccaccccgcggagcacgtacccagggaccgcagccctgacgtgtctcgcctctcctcagtcgtgtgtactgtacccaagcctgagtgttaatttaactctatgttgtccgccgtgtagacatccgaggtcatttgttgcgttgaattatctgaccatccttttttactgtgactcttcccattctctttggcaagaagtccccttctcgcccccaaaccagcaagggactcccccacctgggtctgtgccctgccccgcgctgggggccgagtccttgaatgtggcttcaggggctcctgtcctgacggttgcgtccgggggaggggaaggaagggccgctgtcgccaaggttttctctcccagaaccccgtgggccgccgcgatggccctcaagatggtgaagggcagcatcgaccgcatgttcgacaagaatctgcaggacttggtccgcggcatccgtaaccacaaggaggacgaggcaaaatacatatctcagtgcattgatgagatcaagcaggagctgaagcaggacaacatcgcggtgaaggcgaacgcggtctgcaagctgacgtatttacagatgttgggatacgacatcagctgggccgccttcaacatcatagaagtgatgagtgcctccaagttcaccttcaagcgaattggctacctcgctgcttcccagagctttcacgaaggcaccgacgtcatcatgctgaccaccaatcagatccgtaaggacttgagcagccccagccagtacgacacaggtgttgcactgacgggtctgtcctgcttcgtcaccccagaccttgccagagacctggcaaatgacatcatgacactgatgtcacacaccaagccctacatcaggaagaaggctgtgctgatcatgtacaaggtgttcctgaagtaccccgagtcgctgcgccctgcctttccccggctgaaggagaagctggaggaccccgaccccggggttcagtcggctgccgtcaatgtcatctgcgagctggccagacgcaaccctaagaactacctgtccctggccccgctctttttcaagctgatgacgtcctccaccaacaactgggtcctcatcaagatcatcaagctgttcggtgctcttactcctttggaaccgcggctgggcaagaagctgatcgagcccctcaccaatctcatccacagcacgtctgccatgtctctcctctatgaatgtgtgaacaccgtgattgcagtgctcatctcgctgtcctccggcatgcccaaccacagcgccagcatccagctttgtgttcagaaattaaggatattgatcgaggactccgatcagaacttgaagtacctggggctgctggcaatgtccaagatcctgaagacccaccccaagtccgtgcagtcccacaaggacctcatcctgcagtgcctggacgacaaggacgagtccatccggctgcgggccctggacctgctctatgggatggtgtccaagaagaacctgatggagatcgtgaagaagctgatgacccacgtagacaaggcagagggtaccacctaccgtgacgagctgctcaccaagatcattgacatctgcagccagtccaactaccagtacatcaccaacttcgagtggtacatcagcatcctggtggagctgacccggctggagggcacacggcacggccacctcatcgccgcccaaatgctggacgtggccatccgcgtgaaggccatccgcaagttcgccgtgtcccagatgtctgcgctgcttgacagtgcacacctgctggccagcagcacccagcggaacgggatctgtgaggtgctgtacgctgccgcctggatctgcggggagttctcagagcatctgcaggaaccacaccacactttggaggccatgctgcggcccagagtcaccacgctgccaggccacatccaggccgtgtatgtgcagaacgtggtcaagctctacgcctccatcctgcagcagaaggagcaggccggggaggcagagggcgctcaggccgtcacccagctcatggtggaccggctgccccagtttgtgcagagcgcagacctggaggtgcaggagcgggcgtcctgcatcctgcagctggtcaagcacatccagaagcttcaggccaaggacgtgcctgtggcagaggaggtcagcgctctctttgctggggagctgaacccagtggcccccaaggcccagaagaaggttccagtccccgaaggcctggacctggacgcctggatcaatgagccactctcggacagcgagtcagaggacgagaggcccagggccgtcttccacgaggaggagcagcggcgtcccaagcaccggccgtcggaggcggacgaggaagagctggctcggcgccgagaggcccggaagcaggagcaggccaacaaccccttctacatcaagagctcgccatcgccacagaagcggtaccaggacaccccgggcgtggagcacattcccgtggtgcagattgacctctccgtccccttgaaggttccagggctgcctatgtcagatcagtatgtgaagctggaggaggagcggcggcaccggcagaagctggagaaggacaagaggaggaaaaagaggaaggagaaggagaagaagggcaagcgccgccacagctcgctgcccacggagagcgacgaggacatcgcccctgcccagcaggtggacatcgtcacagaggagatgcctgagaatgctctgcccagcgacgaggatgacaaagaccccaacgacccctacagggctctggatattgacctggataagcccttagccgacagcgagaaactgcctattcagaaacacagaaacaccgagacctcaaaatcccctgagaaggacgttcccatggtagaaaagaagagcaagaaacccaagaagaaagagaaaaaacacaaagagaaagagagagacaaggagaagaagaaggagaaggagaagaagctgaggacctggacttctggctgtctaccaccctgggattcctgaggcccgatggacaagggacctcaaagatggaaaagagatcgacgcgctcgacaagagcactgagagagagcacggcgtaaggaacgaaggctctcacggcagcaggagaccgctgaatgacctggacgtctctgaacttgggcaacaccggcgctagcgacaatgacgacatcatagagtggaaacccaagccacacaacaaagaaccgaacggatgggggacacacacgggatcggggaatatgcctacacggcagccctcatgccgcagacgcgagcagccaaccacacaagaaagggaaagacaaaaacgagaggccaactagaaacgtcaggcacaagcgcgcccggaacagaggagaccacacacacacatgcccgagataggcgagaatagaaagcaccaagatcccgagaaaacgcaagccagtcggaggaaggagggcagcacgacaaggcagacgcgcgggcaactcacacgttacatcatccgccaagagagacaagggtggcgcaagctccatgatagagcacgaacgaccgcacgaagagtacgcagcaacaccagggacagtagccgccgagtacgccataagagtggaggcaggacagagatatagtcgagaaaccagcgagggcga

SEQ ID NO: 30 - protein, 1589 aa, Homo sapiens [sequence omitted]

SEQ ID NO: 31 - DNA, 3712 bp, Homo sapiens
tggttagatccctcaacgctttatacttcctcatctgcctccaaaaataactaaacatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaataagttggttctagcgcagttttttttttcttgtctataaagcatttaacccccctgtacacaactcactccttttaaagaaaaaaattgaaatgtaaggctgtgtaagatttgtttttaaactgtacagtgtctttttttgtatagttaacacactaccgaatgtgtctttagatagccctgtcctggtggtattttcaatagccactaaccttgcctggtacagtatgggggttgtaaattggcatggaaatttaaagcaggttcttgttggtgcacagcacaaattagttatatatggggatggtagttttttcatcttcagttgtctctgatgcagcttatacgaaataattgttgttctgttaactgaataccactctgtaattgcaaaaaaaaaaaaaagttgcagctgttttgttgacattctgaatgcttctaagtaaatacaattttttttattagtattgttgtccttttcataggtctgaaatttttcttcttgaggggaagctagtcttttgcttttgcccattttgaatcacatgaattattacagtgtttatcctttcatatagttagctaataaaaagcttttgtctacacaccctgcatatcataatgggggtaaagttaagttgagatagttttcatccataactgaacatccaaaatcttgatcagttaagaaatttcacatagcccacttacatttacaaactgaagagtaatcaatctactcaaagcatgggattattagaatcaaacattttgaaagtctgtccttgaaggactaatagaaaagtatgttctaacctttacatgaggactctattctttaactcccattaccatgtaatggcagttatattttgcagttcccacattaaagaagacctgagaatgtatccccaaaagcgtgagcttaaaatacaagactgccatattaaattttttgttgacattagtctcagtgaagactatgaaaatgctggctatagatgtcttttcccatttatctaaatatggactgctcaggaaacgagactttccattacaagtatttttaattaattgggccagcttttcaaacaaagatgccacattcaaaatagggtatattttcctatattacggtttgcccctttataaatccaagtagataggaagaaagaagacaaactttgcatctcagtatgaattattcaatttatttgaatgatttttctttacaaaacaaactcattcattagtcatgtttatctgcttaggagtttagggaacaatttggcaattttgtggttttcgagattatcgttttcttaaagtgccagtattttaaaatagcgttcttgtaattttacacgcttttgtgatggagtgctgttttgttatataatttagacttggattctttccatttgcatttgtttatgtaatttcaggaggaatactgaacatctgagtcctggatgatactaataaactaataattgcagaggttttaaatactagttaaatggctttcacttaagaacttaagattttgttacatatttttaaatcttgtttctaataatacctcttagcagtaccttttaaataagtataagggatggcaaagtttttccctttaaaaatactcactttatgcttataaataggttaatgggctgataaaaggttttgtcaaacattgcaagtattcggtgctatatataaaggaggaaaaactagttttactttcagaatgatttaaacaagatttttaaaaacaagatacatgcaagcgaacagcagggttagtgataggctgcaattgtgtcgaacatcagattttttgttaagaggagcaaatgactcaatctgatttagatggaagtttctactgtatagaaatcaccattaatcaccaacattaataattctgatccatttaaaatgaattctggctcaaggagaatttgtaactttagtaggtacgtcatgacaactaccatttttttaagatgttgagaatgggaacagtttttttagggtttattcttgaccacagatcttaagaaaatggacaaaacccctcttcaatctgaagattagtatggtttggtgttctaacagtatcccctagaagttggatgtctaaaactcaagtaaatggaagtgggaggcaatttagataagtgtaaagccttgtaactgaagatgattttttttagaaagtgtatagaaactattttaatgccaagatagttacagtgctgtggggtttaaagactttgttgacatcaagaaaagactaaatctataattaattgggccaacttttaaaatgaagatgctttttaaaactaatgaactaagatgtataaatcttagtttttttgtattttaaagataggcatatggcatattgattaacgagtcaaatttcctaactttgctgtgcaaaggttgagagctattgctgattagttaccacagttctgatgatcgtcccatcacagtgttgttaatgtttgctgtatttattaattttcttaaagtgaaatctgaaaaatgaaatttgtgtgtcctgtgtacccgaggggtaatgattaaatgataaagataagaaaagcgcccatgtaacacaaactgccattcaacaggtatttcccttactacctaaggaattgtaaccattgctcagacattgtaggatttaactatgttgaaaactacaggagaggccgggcgcagtggctcacgcctgtaatcccagcactttgggaggccaaggcgggcagatcacgaggtcaggagattgagaccatcctggctaacgtggtgaaaccccgcctctactaaaaatacaaaaaattagccaagcgtggtgctgggcgcctgtagtcccagtaactcaggaggctgaggcaggagaatggcgtgaacccgggaggcggaggttgcagtgagccgagattgtgccactgcactccagcctgggtgacagagcaagactccatctc

SEQ ID NO: 32 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 33 - DNA, 1678 bp, Homo sapiens
ggggggggcctgtcccggaggtggcggcggcgccatcttggcgaaggggggatcaggaagtgcggaccgcggcggcggcggcggcggcggcggcggcggaccggagcgcaggccggaggtcccggcccgccggccccggagcggaggaacggaaggatgcagcagccgcagccgcaggggcagcagcagccggggccggggcagcagctggggggccagggggcggcgccgggggccgggggcggcccaggggggggcccggggccggggccctgcctgaggcgagagctgaagctgctcgagtccatcttccaccgcggccacgagcgcttccgcattgccagcgcctgcctggacgagctgagctgcgagttcctgctggctggggccggaggggccggggcgggggccgcgcccggaccgcatctccccccacgggggtcggtgcctggggatcctgtccgcatccactgcaacatcacggagtcataccctgctgtgccccccatctggtcggtggagtctgatgaccctaacttggctgctgtcttggagaggctggtggacataaagaaagggaatactctgctattgcagcatctgaagaggatcatctccgacctgtgtaaactctataacctccctcagcatccagatgtggagatgctggatcaacccttgccagcagagcagtgcacacaggaagacgtgtcttcagaagatgaagatgaggagatgcctgaggacacagaagacttagatcactatgaaatgaaagaggaagagccagctgagggcaagaaatctgaagatgatggcattggaaaagaaaacttggccatcctagagaaaattaaaaagaaccagaggcaagattacttaaatggtgcagtgtctggctcggtgcaggccactgaccggctgatgaaggagctcagggatatataccgatcacagagtttcaaaggcggaaactatgcagtcgaactcgtgaatgacagtctgtatgattggaatgtcaaactcctcaaagttgaccaggacagcgctttgcacaacgatctccagatcctcaaagagaaagaaggagccgacttcattctacttaacttttcctttaaagataactttccctttgacccaccatttgtcagggttgtgtctccagtcctctctggagggtatgttctgggcggaggggccatctgcatggaacttctcaccaaacagggctggagcagtgcctactccatagagtcagtgatcatgcagatcagtgccacactggtgaaggggaaagcacgagtgcagtttggagccaacaaatctcaatacagtctgacaagagcacagcagtcctacaagtccttggtgcagatccacgaaaaaaacggctggtacacacccccaaaagaagacggctaaccctgggcagagcacagcatcgtcgggaccagactcgtctcaggccagttgcagccttctcagccaaacgccgaccaaggacaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaacctgacccatctcagaagcgcgcgg

SEQ ID NO: 34 - protein, 474 aa, Homo sapiens
GGGLSRRWRRRHLGEGGIRKCGPRRRRRRRRRRTGAQAGGPGPPAPERRNGRMQQPQPQGQQQPGPGQQLGGQGAAPGAGGGPGGGPGPGPCLRRELKLLESIFHRGHERFRIASACLDELSCEFLLAGAGGAGAGAAPGPHLPPRGSVPGDPVRIHCNITESYPAVPPIWSVESDDPNLAAVLERLVDIKKGNTLLLQHLKRIISDLCKLYNLPQHPDVEMLDQPLPAEQCTQEDVSSEDEDEEMPEDTEDLDHYEMKEEEPAEGKKSEDDGIGKENLAILEKIKKNQRQDYLNGAVSGSVQATDRLMKELRDIYRSQSFKGGNYAVELVNDSLYDWNVKLLKVDQDSALHNDLQILKEKEGADFILLNFSFKDNFPFDPPFVRVVSPVLSGGYVLGGGAICMELLTKQGWSSAYSIESVIMQISATLVKGKARVQFGANKSQYSLTRAQQSYKSLVQIHEKNGWYTPPKEDG

SEQ ID NO: 35 - DNA, 7588 bp, Homo sapiens
cggtctctttgcaaatgtcaatgtatttttttccttcaatatactgtatctattcctctattattaagttctgagcttgctttaccttgtccgctatttaaaacattctgatttgaaagtatcaccaacagcttatgtgattttccaaatttctcaaaatacagttgtcaccattagtcacatgaagtctgctgccgccgccgcagccgcagctactgtgacttctccgattgtgtgagctttgttggagcctgcgtacgtggatttatcgctgccacggtctgcgtagctccagaggtttaaccataggatagagaaaccaggaatgtaatgaggaaatcaaaatggatccaagtatgggtgtgaattctgttaccatttctgttgagggtatgacttgcaattcctgtgtttggaccattgagcagcagattggaaaagtgaatggtgtgcatcacattaaggtatcactggaagaaaaaaatgcaactattatttatgaccctaaactacagactccaaagaccctacaggaagctattgatgacatgggctttgatgctgttatccataatcctgaccctctccctgttttaactgacaccttgtttctgactgttacggcgtcactgactttgccatgggaccatatccaaagcacattgctgaagaccaagggtgtgacagacattaaaatttaccctcagaaaagaactgtagcagtgacaataatcccttctatagtgaatgccaatcagataaaagagctggttccagaactcagtttagatactgggacactggagaaaaagtcaggagcttgtgaagatcatagtatggctcaagctggtgaagtcgtgctgaagatgaaagtggaagggatgacctgccattcatgtactagcactattgaaggaaaaattgggaaactgcaaggtgttcagcgaattaaagtctccctggacaatcaagaagctactattgtttatcaacctcatcttatctcagtagaggaaatgaaaaagcagattgaagctatgggctttccagcatttgtcaaaaagcagcccaagtacctcaaattgggagctattgatgtagaacgtctaaagaacacaccagttaaatcctcagaagggtcacagcaaaggagtccatcatataccaatgattcaacagccactttcatcattgatggcatgcattgtaaatcatgtgtgtcaaatattgaaagtactttatctgcactccaatatgtaagcagcatagtagtttctttagagaataggtctgccattgtgaagtataatgcaagctcagtcactccagaatccctgagaaaagcaatagtggctgtatcaccggggctatatagagttagtatcacaagtgaagttgagagtacctcaaactctccctccagctcatctcttcagaagattcctttgaatgtagttagccagcctctgacacaagaaactgtgataaacattgatggcatgacttgtaattcctgtgtgcagtctattgagggtgtcatatcaaaaaagccaggtgtaaaatccatacgagtctcccttgcaaatagcaatgggactgttgagtatgatcctctactaacctctccagaaacgttgagaggagcaatagaagacatgggatttgatgctaccttgtcagacacgaatgagccgttggtagtaatagctcagccttcatcggaaatgccgcttctgacttcaactaatgaattttatactaaagggatgacaccagttcaagacaaggaggaaggaaagaattcatctaagtgttacatacaggtcactggcatgacttgcgcttcctgtgtagcaaacattgaacggaatttaaggcgggaagaaggaatatattctatacttgtggccctgatggctggcaaggcagaagtaaggtataatcctgctgttatacaacccccaatgatagcagagttcatccgagaacttggatttggagccactgtgatagaaaatgctgatgaaggagatggtgttttggaacttgttgtgaggggaatgacgtgtgcctcctgcgtacataaaatagagtctagtctcacaaaacacagagggatcctatactgctccgtggccctggcaaccaacaaagcacatattaaatatgacccagaaattattggtcctagagatattatccatacaattgaaagcttaggttttgaagcttctttggtcaagaaggatcggtcagcaagtcacttagatcataaacgagaaataagacaatggagacggtcttttcttgtgagtctgtttttctgtattcctgtaatggggctgatgacatatatgatggttatggaccaccactttgcaactcttcaccataatcaaaacatgagtaaagaagaaatgatcaaccttcattcttctatgttcctggagcgccagattcttccaggattgtctgttatgaatttgctgtcctttttattgtgtgtacctgtacagtttttcggaggctggtacttctacattcaggcttataaagcactgaagcataagacagcaaatatggacgtactgattgtgctggcaaccaccattgcatttgcctactctttgattattcttctagttgcaatgtatgagagagccaaagtgaaccctattactttctttgacacaccccctatgctgtttgtgtttattgcactaggccgatggctggaacatatagcaaagggcaaaacatcagaggctcttgcaaagttaatttcactacaagctacagaagcaactattgtaactcttgattctgataatatcctcctcagtgaagaacaagtggatgtggaacttgtacaacgtggagatatcattaaagtagttccaggaggcaaatttccagtggatggtcgtgttattgaaggacattctatggtagatgagtccctcatcacaggggaggcaatgcctgtggctaagaaacctggcagcacagtgattgctggttccattaaccagaacgggtcactgcttatctgcgcaacacatgttggagcagacacaaccctttctcaaattgtcaaacttgtggaagaggcacaaacatcaaaggctcctatccagcagtttgcagacaaactcagtggctattttgttccttttattgtttttgtttccattgccaccctcttggtatggattgtaattggatttctgaattttgaaattgtggaaacctactttcctggctacaatagaagtatctcccgaacagaaacgataatacgatttgctttccaagcctctatcacagttctgtgtattgcatgtccctgttcactgggactggccactccaactgctgtgatggtgggtacaggagtaggtgctcaaaatggcatactaataaaaggtggagagccattggagatggctcataaggtaaaggtagtggtatttgataagactggaaccattactcacggaaccccagtggtgaatcaagtaaaggttctaactgaaagtaacagaatatcacaccataaaatcttggccattgtgggaactgctgaaagtaacagtgaacaccctctaggaacagccataaccaaatattgcaaacaggagctggacactgaaaccttgggtacctgcatagatttccaggttgtgccaggctgtggtattagctgtaaagtcaccaatattgaaggcttgctacataagaataactggaatatagaggacaataatattaaaaatgcatccctggttcaaattgatgccagtaatgaacagtcatcaacttcgtcttccatgattattgatgcccagatctcaaatgctcttaatgctcagcagtataaagtcctcattggtaaccgggagtggatgattagaaatggtcttgtcattaataacgatgtaaatgatttcatgactgaacatgagagaaaaggtcggactgctgtattagtagcagttgatgatgagctgtgtggcttgatagccattgcagacacagtgaagcctgaagcagaactggctatccatattctgaaatctatgggcttagaagtagttctgatgactggagacaacagtaaaacagctagatctattgcttctcaggttggcattactaaggtgtttgctgaagttctaccttctcacaaggttgctaaagtgaagcaacttcaagaggaggggaaacgggtagcaatggtgggagatggaatcaatgactccccagctctggcaatggctaatgtgggaattgctattggcacaggcacagatgtagccattgaagcagctgatgtggttttgataaggaatgatcttctggatgtagtggcaagtattgacttatcaagaaagacagtcaagaggattcggataaattttgtctttgctctaatttataatctggttggaattcccatagctgctggagtttttatgcccattggtttggttttgcagccctggatgggatctgcagcaatggctgcttcatctgtttctgtagtactttcttctctcttccttaaactttacaggaaaccaacttacgagagttatgaactgcctgcccggagccagataggacagaagagtccttcagaaatcagcgttcatgttggaatagatgatacctcaaggaattctcctaaactgggtttgctggaccggattgttaattatagcagagcctctataaactcactactgtctgataaacgctccctaaacagtgttgttaccagtgaacctgacaagcactcactcctggtgggagacttcagggaagatgatgacactgcattataaaaggccatggagagtgctgccagtttaacttgtcatgcactgacacagcattcatgatgttaccttcacttttcaaaatattgtagaaggatttttctcatgctcttatattagggattctatttgagttgcgtttatctgttggcaaaaatatctttttcaaggcatcagctctgaacctagctttatttaaactgaatttccagtatatttttgttttcactaacaacagataaggtagagcagtgaggtttacaacaagccctacaattagagattgctgaactgctgctaaagtgattttttttttatttgaccaaaaaaaaaaaggcccaagaagaagaaaatgaaaaatttgaagatttgagagcatgaagatattcatgcttttgaactcaaaatattgaagatactctcaagcctgtatccctgccccactggggagcaatgactttcaaagcactgtgtataaaacatctagttttagaagggaaacagttgaaactgtttaaaaatagatgtgccttatttattgcaggctttctttcccccattctccctgcatccttgtccttgcaggtgcttttttagatgctccaatatgtcttcttttgttattttctttcgagctaaccagtttaggtggtttttcattgattaaaaataactgacaactgttctaatattttgctcctttttaaattttgtagctcaaaagaccttaaaggtctgtagggttccctgcctcccatctttccactgttgtaaaaagtatatcaaattattccttcaagtttcctagctctgtgctcagtttcagttcactcctgccaagttggactctaagttattcttcatgtagtctgctgatctcagtctggaaacttaacattatgagccttttctgctcaaaaaattttcaaagattaaaactattatacatatacaggtcatataaaattacctggattcactaaatttgtttgttgttgttgttgttgttgttgttgttgttgagacagagtcttgttttgcagcccaggctggagtgcagtggcaccatcttggctcactgcaacctctgcctaccggattcaaggaattctccctgcctcagcctcctgagtagctaggattacaggtgcctgccaccacacccggctaattttcatatttttcagtagagacggggtttcgccatgttggctagcctggtcttaaactcctaacctcaggtgatccacccgccttggcctcccaaagtgctgggattacaggtgtgagcctccatgcccagcctaaatttgtattttttgaattgagtataatcactttgctagtatatataatttaatagattttatttatcttttagtgtttcagataacctctcaaaaagactttaaaataatgctattacacaaagctgcatttaccaaaaaatacagtaaaatcataatacagaaactaaaatttccctaggttatgacgctttttagctaaatatatactcttctctagtttaaaacatttgaacttgcctagttagtgtggttggcaaatttaggagcttgttcccattgccaaatggatttagaaattcccttgtgagtgcctggtagctaatacactggtcagagatctggtacttgtaagactatttaaatttctttgttagttgcaagatggatttcatatgcagaatatgtaaatgaagaggactcataagtaaattcctaacattttgttcccattaccagaagcaaagctgctgctaacccaacatctggcacataggatttgtactcggtaaatgttagttcttttctccccttgaggtcagtaataaatacaaaaaaatcatttttctagagcagagtcttaaaatcaggtgggggtaggggatggagttcttcctttcctaccccttttctcttttatcctttcatatatacacatgcaaagtttacaaccttattccatgctgtctttcagatttagaaaagatctaatttctgtctcagctgtcttaaagagagaactgaagcttttgatgaaggtgctattaatctagaaaggcaaacccatttcactgaaatatcaatgggtttgcatatctaggccctttttttagaccaatgcctatgccatcctccatgctttcagtttgagttttattatttattattttaattccagtggcccatcttataatacaacttgtttcttctagaagacagagctgatagggtaaatgttgaaaaaagaagcatgcctccttctccctcccacccacctcaagcagttgaacacagccagttattcttccattattatgtgtaccttggagtcatcctcttggtcttgtattcatattgtgggacagtgggaatagcagcttgtagtattgaaataatcaaagagataatttcagctcttacaacaagaacagaaaacatgctaattagaaaaagtcctgctttaagtaatatgtagccacatttgaatcctctaccacaagtcttttttgcaatcttgaactttcattagcttcaaagagaagctgtatttacaggagaaagaggtgattatggagggaatcaaaaatactgctttcagttagtagctagctttaagtcaggagttagtaatgagaaattttataaatgtgtatttctgtgtattcaccatcctattttttatcccccccatttttttgtccattccccttacattccttt

SEQ ID NO: 36 - protein, 1500 aa, Homo sapiens
MDPSMGVNSVTISVEGMTCNSCVWTIEQQIGKVNGVHHIKVSLEEKNATIIYDPKLQTPKTLQEAIDDMGFDAVIHNPDPLPVLTDTLFLTVTASLTLPWDHIQSTLLKTKGVTDIKIYPQKRTVAVTIIPSIVNANQIKELVPELSLDTGTLEKKSGACEDHSMAQAGEVVLKMKVEGMTCHSCTSTIEGKIGKLQGVQRIKVSLDNQEATIVYQPHLISVEEMKKQIEAMGFPAFVKKQPKYLKLGAIDVERLKNTPVKSSEGSQQRSPSYTNDSTATFIIDGMHCKSCVSNIESTLSALQYVSSIVVSLENRSAIVKYNASSVTPESLRKAIVAVSPGLYRVSITSEVESTSNSPSSSSLQKIPLNVVSQPLTQETVINIDGMTCNSCVQSIEGVISKKPGVKSIRVSLANSNGTVEYDPLLTSPETLRGAIEDMGFDATLSDTNEPLVVIAQPSSEMPLLTSTNEFYTKGMTPVQDKEEGKNSSKCYIQVTGMTCASCVANIERNLRREEGIYSILVALMAGKAEVRYNPAVIQPPMIAEFIRELGFGATVIENADEGDGVLELVVRGMTCASCVHKIESSLTKHRGILYCSVALATNKAHIKYDPEIIGPRDIIHTIESLGFEASLVKKDRSASHLDHKREIRQWRRSFLVSLFFCIPVMGLMTYMMVMDHHFATLHHNQNMSKEEMINLHSSMFLERQILPGLSVMNLLSFLLCVPVQFFGGWYFYIQAYKALKHKTANMDVLIVLATTIAFAYSLIILLVAMYERAKVNPITFFDTPPMLFVFIALGRWLEHIAKGKTSEALAKLISLQATEATIVTLDSDNILLSEEQVDVELVQRGDIIKVVPGGKFPVDGRVIEGHSMVDESLITGEAMPVAKKPGSTVIAGSINQNGSLLICATHVGADTTLSQIVKLVEEAQTSKAPIQQFADKLSGYFVPFIVFVSIATLLVWIVIGFLNFEIVETYFPGYNRSISRTETIIRFAFQASITVLCIACPCSLGLATPTAVMVGTGVGAQNGILIKGGEPLEMAHKVKVVVFDKTGTITHGTPVVNQVKVLTESNRISHHKILAIVGTAESNSEHPLGTAITKYCKQELDTETLGTCIDFQVVPGCGISCKVTNIEGLLHKNNWNIEDNNIKNASLVQIDASNEQSSTSSSMIIDAQISNALNAQQYKVLIGNREWMIRNGLVINNDVNDFMTEHERKGRTAVLVAVDDELCGLIAIADTVKPEAELAIHILKSMGLEVVLMTGDNSKTARSIASQVGITKVFAEVLPSHKVAKVKQLQEEGKRVAMVGDGINDSPALAMANVGIAIGTGTDVAIEAADVVLIRNDLLDVVASIDLSRKTVKRIRINFVFALIYNLVGIPIAAGVFMPIGLVLQPWMGSAAMAASSVSVVLSSLFLKLYRKPTYESYELPARSQIGQKSPSEISVHVGIDDTSRNSPKLGLLDRIVNYSRASINSLLSDKRSLNSVVTSEPDKHSLLVGDFREDDDTAL

SEQ ID NO: 37 - DNA, 2660 bp, Homo sapiens
caggagaactacatagcccagcatgcaacagtctcaactttttcacggacactacatttcccagaaggcagttcgcaccgcagtgttttctgggatgggaaccacgccgcttcccagtctctgtgcgaggcgtgaagcgcggacctttcaacaagggctttattaattctcacgctgcggccctggaaagcgatggaggtggcggctaattgctccctacgggtgaagagacctctgttggatccccgcttcgagggttacaagctctctcttgagccgctgccttgttaccagctggagcttgacgcagctgtggcagaggtaaaacttcgagatgatcaatatacactggaacacatgcatgcttttggaatgtataattacctgcactgtgattcatggtatcaagacagtgtctactatattgatacccttggaagaattatgaatttaacagtaatgctggacactgccttaggaaaaccacgagaggtgtttcgacttcctacagatttgacagcatgtgacaaccgtctttgtgcatctatccatttctcatcttctacctgggttaccttgtcagatggaactggaagattgtatgtcattggaacaggtgaacgtggaaatagcgcttctgaaaaatgggagattatgtttaatgaagaacttggggatccttttattataattcacagtatctcactgctaaatgctgaagaacattctatagctaccctacttcttcgaatagagaaagaggaattggatatgaaaggaagtggtttctatgtttctctggagtgggtcactatcagtaagaaaaatcaagataataaaaaatatgaaattattaagcgtgatattctccgtggaaagtcagtgccacattatgctgctattgagcctgatggaaatggtctaatgattgtatcctacaagtctttcacatttgttcaggctggtcaagatcttgaagaaaatatggatgaagacatatcagagaaaatcaaagaacctctgtattactggcaacagactgaagatgatttgacagtaaccatacggcttccagaagacagtactaaggaggacattcaaatacagtttttgcctgatcacatcaacattgtactgaaggatcaccagtttttagaaggaaaactctattcatctattgatcatgaaagcagtacatggataattaaagagagtaatagcttggagatttccttgattaagaagaatgaaggactgacctggccagagctagtaattggagataaacaaggggaacttataagagattcagcccagtgtgctgcaatagctgaacgtttgatgcatttgacctctgaagaactgaatccaaatccagataaagaaaaaccaccttgcaatgctcaagagttagaagaatgtgatattttctttgaagagagctccagtttatgcagatttgatggcaatacattaaaaactactcatgtggtgaatcttggaagcaaccagtaccttttctctgtcatagtggatcctaaagaaatgccctgcttctgtttgcgccatgatgttgatgccctactctggcaaccacactccagcaaacaagatgatatgtgggagcacatcgcaactttcaatgctttaggctatgtccaagcatcaaagagagacaaaaaattttttgcctgtgctccaaattactcgtatgcagccctttgtgagtgccttcgtcgagtattcatctatcgtcagcctgctcccatgtccactgtactttacaacagaaaggaaggcaggcaagtaggacaggttgctaagcagcaagtagcaagcctagaaaccaatgatcctattttaggatttcaggcaacaaatgagagattatttgttcttactaccaaaaacctctttttaataaaagtaaatacagagaattaattattctaacatattggcctctttgtactggaaaagtattcagtggtacctggaggtctggacagttatactgtaacctcttaagttttaatgtgctaaatatatcttgtatgattttttattttttaataacattggaaatatattcaagagattatgattctgtaaagctgtggaatgaagctgcagatttagagaacattggcttctgaaaaaaaaaaagagtgaagatagtactagcaagtatacttattttttaaaacaggctagaatctcatgttttatatgaaagatgtacaattcagtgtttaaaaataaaaatatttattgtgtaaaaaaaaaaaaaaaaaaaaaaaaaaaaaaaaaaaaaaaaaaaaaaaaaaaacttaaataaatttagtcgccaaattcgtcacagagccaaattcaactctaccttactcccagcaaacttttcccaaacaatttacccaaaaatagtctacctgttagaaattgaaaattgtctccaaaaatttgggccctctagggattgccgaaagatttttcccaggcgtatatatccaagggcttaaccccaataccttttcgataatggaattacttcaaaaaagttctctcagggtacgccaaatcttaaaacccccaaatctagccgcttctacctaacagtttattagaccactccgttgtttctcaccccttttggtgcggtg

SEQ ID NO: 38 - protein, 583 aa, Homo sapiens [sequence omitted]

SEQ ID NO: 39 - DNA, 7052 bp, Homo sapiens
cggratkcgcggccggsgtckascggcgctgctggaagatggcgagcggccgggacgagcggccgcattgcgtagggcggctcctgttgctcatgtgcctgctgctgctggggagctcggcccgggcggctcacatcaagaaggcggaggcgactaccacaactacgagcgcgggcgcgaggggccgagggcagttcgaccgctactaccacgaagaggagttggagtcggcgctgagggaggcggcggccgcgggcctccccggcctggcccgcctctttagcatcggccgctcggtggaaggccggccgctgtgggtgcttcgcctcaccgccggcctggggtcgctaatccctgagggcgacgcggggcctgacgctgccgggcccgacgctgcggggccgctgctgcccggccggccccaggtgaagctggtgggcaacatgcatggcgacgagaccgtgtcgcgccaggtgttgatctacttggcccgcgagctggcggccggctaccgccgcggggacccgcgcctggtccgcctgctcaacaccaccgacgtgtacctgctgcccagcctcaaccccgatggcttcgagcgtgcccgcgagggcgactgtggcttcggcgacggcggcccgtccggggccagcggccgcgacaatagtcgcggccgcgacctcaaccgaagctttcccgaccagtttagcaccggcgaaccccccgccctggacgaggtgcccgaggtgcgcgccctcatcgagtggatccgcaggaacaagtttgtgctttctggaaatctgcatggtggctcagtggtagcaagctatccttttgatgattctccagaacataaggccactggaatctatagcaaaacctcagatgatgaagtatttaaatacttggcaaaagcttatgcttcaaaccaccccataatgaaaactggtgagcctcattgtccaggagatgaagacgagactttcaaagatggaatcacaaacggcgcacattggtatgatgtggaaggtggtatgcaagattacaattatgtgtgggccaactgttttgagatcacattagaactgtcttgttgcaagtacccacctgcttcacagcttcgacaggaatgggagaacaatcgtgagtctttgatcacattgattgaaaaggttcacattggagtgaaaggatttgttaaagattccataacaggatctgggttagagaatgcaaccatctcagtggctggtattaatcataatatcacaacaggcagatttggtgatttctaccgattacttgttcctggaacttacaaccttacagtagttttaactgggtatatgccattgactgttactaatgtagtggtgaaagaaggaccagccacagaggtggatttttctcttaggccaactgtaacttcagtaatccctgacacgacagaggctgtatcaactgctagcacagttgctatacctaatattctttctggaacatcatcctcctaccagccaattcagccaaaggactttcaccaccaccatttccctgatatggaaatcttcttgagaaggtttgccaatgaatatcctaacattacccggctttattccttgggaaaatcagtagagtcaagagaactttatgtgatggagatatctgataatccgggtgtccatgaaccaggtgaaccagaatttaagtacattggaaatatgcatggaaatgaagtggttggaagagaactgctgttgaacctcatagaatacctttgtaagaactttggaacagaccctgaagtcacagatttggttcataacactagaattcaccttatgccatccatgaatcctgatgggtatgaaaagtcccaggaaggagattcaataagtgtaattggcagaaacaacagcaacaactttgacctgaaccgaaatttcccagaccagtttgttcagatcacagatcctacgcaaccagaaactattgctgtaatgagctggatgaagtcctatccatttgtactttcagcaaacctgcatggaggttctttggtggttaactacccttttgatgatgatgaacaaggacttgccacatatagtaaatcaccagatgatgctgtgttccaacaaatagcactttcttattccaaggaaaattcccagatgtttcaaggtagaccttgcaagaatatgtatcctaatgaatattttcctcatggaataacaaatggagctagttggtataatgtgccaggaggaatgcaggactggaactatttacaaacaaattgctttgaagtgactattgaactaggttgtgtgaaatatccacttgagaaagagctgccaaacttttgggaacagaatcgaagatcactaatccagtttatgaaacaggttcatcagggcgtcagaggatttgttctagatgccacagatggcaggggtatattaaatgccaccattagtgttgctgagattaatcacccagtgactacttacaaaactggagattactggcgtctcttggttccaggaacttataaaatcacagcatctgctcgagggtataatccagttaccaagaatgtgactgtcaagagtgaaggcgctattcaggtcaacttcacacttgttcgatcctcaacagattcaaacaatgaatcaaagaaaggaaaaggggctagcagcagcaccaatgatgccagtgatccaactactaaagagtttgaaactttaattaaagacctttcagcggagaatggtttggaaagcctcatgttacgctcctcctcaaatctggctctggctctttatcgataccattcctacaaagacttatcagagtttctgagaggacttgtaatgaactatccacatattacaaatcttaccaatttgggacagagcactgaatatcgtcacatttggtcccttgaaatctccaataagcccaatgtatctgagcctgaagaaccaaagattcgttttgttgctggtatccatggaaatgcgccagttggaactgaactgcttttggctctggcagaatttctctgcctgaactacaaaaagaacccagctgttacccaattggttgacaggactaggattgtgattgtcccttctctaaatccagatgggcgagagagagctcaagagaaagactgtacttcaaaaataggacaaacaaatgctcgtggcaaagatttggatacagacttcacaaataatgcctcccaacctgagaccaaagccatcattgaaaatttgattcaaaaacaggactttagtctttctgttgccttagatggtggttccatgctggtcacatatccttatgacaagccagtacagacagtggaaaataaagagactctgaagcatttggcatctctttatgcaaataatcatccatccatgcacatgggtcagcccagttgcccaaataaatcagatgagaatattccaggaggagtaatgcgtggagcagaatggcatagtcacctgggcagcatgaaggattatagtgtcacctatggccattgtccggaaatcacagtatacacaagctgctgttactttcctagtgctgcacgactcccttccttgtgggcagacaataagagatctcttcttagtatgttagtggaggttcacaagggagttcatggatttgttaaagataagactggaaagccaatctctaaagcagtcattgtacttaatgaaggaataaaggtacaaacaaaagagggaggttatttccatgtactcttagcgccaggtgtccataacattattgccatcgctgatgggtaccagcaacaacattcacaggtctttgtgcatcatgatgcagctagttctgtggtgatagtctttgacacagataaccggatatttggtttgccaagggagcttgtggtaactgtatcaggtgctactatgtcggcattgatcctaacagcttgcattatttggtgcatctgctcaatcaagtctaatagacacaaggatggctttcatcggctcaggcagcatcatgatgagtatgaagatgaaattcgcatgatgtctaccggctccaagaagtccctcctaagccatgagttccaggatgaaacagacactgaagaggaaacattatattctagcaaacattgaaaaacacattttgcatatctcccagcataagtaccaagcaaaattacagttcctcttgggagaacactgcattaagaagagagactctcttgcttcttcaaagagctttgggaaattaaattgctaaatttgtattctctgtgaatttcactggcagttttgaacttcccttccttaaagtactctaaacctttaaaaaaaaatctgatttatgcagcagagatgggacagccactttttctttttaatttaagatgagctatttggagcttatgtaataatggcataaagccaactagaggatgttgtattttgcacatcagatgtttactagtggctttagtatttttctttgttttaaatggccaaaagaatccagaaacattaaggcagggacagcagtcagaatcgacataaagctttaaaaactcaaggttttttcaacctactgaggagtacttttctctagttgttaaatagctggagtttttcttattcaggtttaatggaggttgaattgatttttaaacacatataacagtaggaaatgaataaatgggcttctgcatttggctttctacctgttccaaggctagatcggaactggtagactacgctgtaagcaggatttcactacctctcttaaggtttagcaaacttctaaatagcccattttaagggagaacttactaactttattgtgaaaggtctaaatgcccacttgaatgaagctgagagagagatctagcaaaagctaaaactcatgttgtctatctttgaacttggtaaaaacccacaggtgctgctgcttatatctgtgaagcactagcttattctaggaatgcctgattctttaatattgcctaaatcggaacctttttctatgttgcacacatggttttcagatgacccagccatctacaagatctgaattctactgaaaatatctagaaatgtggaagagacctacttgcacattcttaacctgtatttgaacacaaaatatctatacttcatgctccagcccaagcctataccctgtaatagcatactattattgaaatcgcttgaccggtcttgttcacataggcctctgggagtgatttggttctttgccctaatgtttcatttgacggtctctttttgatcaaccaatttttctaaaagttcagtcgaaagcttttaagtatagcttcctcccttgaaaaaaaatgtaaactatgactgctgagtgataaaacactgtggtgtgaaagtgtcatcttcactgccaatcaggcaaagaccggaaagatttgcattttattatgtctgtcttatcatgcaatggaaatgatgctttttgtaagtatgcatcttaccaatgatgtaacggtttaatacctttgaatgttttaataaccaagttgctgctgaacttatactaaatcaggggaccaaaaaacttgctcttatcttctcaaattgtattctatatccattaatgtatcagttatcccaaagccttcaggtggaggggtttaccaccttcctaggtcgttcaaccaggttttgtgaggaatgcattcaaagtggctttataaaagaagattttctttagcaagaataatgaggtcatgtcatttgttaataagtatctgtgataaatccgtggttcaaggttaagccattctggtattctggtattagcaactgtaaattctgccacctcatacatggaacagagcttgtgggatgctaatagttagtgaagtatacatgatttaatttctaataatctttatgttttctttaaggatggtggtgtattgctctttttcagctttatttttaagagtacagtcaggaaaccaacaaggggcctaagagtggctgcccctgcttgggacattacagcaagtgaaacaaagttaatgtgacaagctttgctttgttatcattggtcttcactagaggataccttttacatgtacttctctcttggatcaaatatgtctttaactgtacatctcagtggctggaggccatgccttttaagcatgtgtaaaatttttaaagaaatgaacatacacatagttattttagtaatatttcctgaaagaaaaaccaaattctgctataagtcttgatcttcaatgaacttttaaataatgcatttagctggaaaacaagactttcccagcttgtgttacctagaagcgtgaatgtataggatacctgactactaagactatattctcagccctgccctgtcttttatttgcgggtctaatctaatattagaatatattaaccgcttaaggcattgaagccatatgggatggggaatgcatttcttcagtgtttctccgagagactttccatttccttggagttatggcggcaagtaagtatcatagtattaagaaatttgcctaaatctgagttgtgcctttctttactcacaaggcatgggctttgtcctggtgatcagtttgtaagccttcttccttcccagctccttaataaaagcaaagtgattgagtaggtaatgttcaaagtgtctgcctgtgtacatgtacttgtattgattatgtagttcagtaagatgtgcccaagtcatttcagaaagaaagacccttcagttttgatgcattttgctgaacacttgggtagtgagtgggatcctatccagttgaggaatgcttgcaatgctcattgaagggatttgctttgggactttgtcatcttccagaaaggaaacatattgtatatttggcccagtgtgattgattgctttatctttggtaacttttacttgaatgggatttgctgaattaatgactattgaatttaaaactaattatgagttgacaaataaataaaaggtagtgttta

SEQ ID NO: 40 - protein, 1389 aa, Homo sapiens [sequence omitted]

(19) United States
(12) Patent Application Publication
(10) Pub. No.: US 2009/0016963 A1
(43) Pub. Date: Jan. 15, 2009

(54) Genes involved in neurodegenerative conditions

(76) Inventors: Dalia Cohen, Winchester, MA (US); Uwe Jochen Dengler, Lorrach (DE); Danny Garza, Norfolk, MA (US); Anju N. Kelkar, Sayville, NY (US); Mary Konsolaki, Westfield, NJ (US); Ho-Juhn Song, Newton, MA (US)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 10/576,051

(22) PCT Filed: Oct. 21, 2004
(86) PCT No.: PCT/EP2004/011926

§ 371 Date: Jul. 24, 2006

(30) Foreign Application Priority Data
Oct. 22, 2003 (US) 60513241

(51) Int. Cl. A61K 39/395; A61K 31/7088; C12Q 1/68; G01N 33/68; A61K 49/00; A61K 48/00; A61P 25/28

(52) U.S. Cl. 424/9.2; 424/139.1; 514/44; 435/6; 436/86

(57) ABSTRACT

The invention discloses suitable gene and polypeptide targets for the development of new therapeutics to treat, prevent or ameliorate neurodegenerative conditions. The invention also relates to methods to treat, prevent or ameliorate said conditions and pharmaceutical compositions therefor, as well as to a method to identify compounds with therapeutic usefulness to treat neurodegenerative conditions.

### FIELD OF THE INVENTION

This invention relates to genes involved in the development and/or progression of neurodegenerative conditions, specifically conditions involving the aberrant metabolism, trafficking or turnover of A beta (Aβ) including, but not limited to, Alzheimer's Disease (AD). The invention also relates to the use of said genes as drug targets for the development of therapeutics useful to treat, prevent or ameliorate said neurodegenerative conditions.

### BACKGROUND OF THE INVENTION

AD is a progressive neurodegenerative disease that results in gradual cognitive and behavioral changes and loss of memory. See Selkoe, Physiol Rev, Vol. 81, No. 2, pp. 741-766 (2001); Selkoe and Podlisny, Annu Rev Genomics Hum Genet, Vol. 3, No. 3, pp. 67-99 (2002). Familial forms of AD have been linked to mutations in the gene that encodes amyloid precursor protein (APP). Differential cleavage of APP leads to production of 40 or 42 amino acid long peptides, designated as Aβ40 and Aβ42. APP mis-sense mutations are clustered around the Aβ cleavage sites and either increase the total production of Aβ-peptides or the Aβ42-/Aβ40-peptide ratio. Although both of these peptides are components of senile plaques (the neuropathological hallmark of AD), overproduction of Aβ42 is conducive to formation of amyloid plaques due to its hydrophobic nature and self-aggregation properties. Evin and Weidemann, Peptides, Vol. 23, No. 7, pp. 1285-1297 (2002).

We have previously developed a fly model for AD by over-expressing the amyloidogenic Aβ42-peptide in the fly eye with the help of the eye-specific GMR promoter. See U.S. Patent No. US 2002 0174446). Presence of the pGMR-Aβ42 construct in the transgenic flies produces a rough-eye phenotype. The eye roughness increases progressively with aging of the flies, mimicking the age dependence of AD. Using this fly model we have conducted a genetic screen to look for modifiers of the Aβ42-dependent rough-eye phenotype. Our screen utilizes a publicly available collection of fly strains carrying independent insertions of the Expression P (EP) element in various regions of the fly genome. See Rorth, Proc Natl Acad Sci USA, Vol. 93, No. 22, pp. 12418-12422 (1996); Rorth et al., Development, Vol. 125, No. 6, pp. 1049-1057 (1998); and Berkley Drosophila Genome Project (BDGP) http://www.fruiffly.org/blast/). The EP element used for making these fly strains consists of the Gal4-activated upstream activating sequences (UAS) element and a minimal hsp70-promoter. Since P elements have a tendency to insert in the 5′ regulatory region of genes [see Liao, Rehm and Rubin, Proc Natl Acad Sci USA, Vol. 97, No. 7, pp. 3347-3351 (2000)], most EP insertions are expected to target genes for directed mis-expression. Therefore, by crossing these EP strains with fly strains containing tissue-specific Gal4 drivers, a desired tissue specific mis-expression of EP-linked genes can be achieved. See Duffy, Genesis, Vol. 34, Nos. 1 and 2, pp. 1-15 (2002). In order to carry out the EP-based genetic screen, we have recombined the eyeless Gal4 driver (eyGal4) [see Sheng et al., Genes Dev, Vol. 11, No. 9, pp. 1122-1131 (1997); and Halder et al., Development, Vol. 125, No. 12, pp. 2181-2191 (1998)] in our Aβ over-expressing fly strain to direct expression of EP-linked genes in the eye. From this genetic screen we can determine genetic interactions that would affect the stability, aggregation, toxicity and/or secretion of the Aβ42-peptide, manifested as modification of the rough-eye phenotype.

Applicants disclose herein surprising evidence suggesting that in our transgenic model, the Aβ42-peptide is secreted by the Drosophila photoreceptor cells. Using this model system, Applicants have discovered and describe herein several new genes involved in the development and/or progression of AD. Thus, it is contemplated herein that these genes and the proteins encoded by these genes may serve as drug targets for the development of therapeutics to treat, prevent or ameliorate neurodegenerative conditions, specifically conditions involving, e.g., the aberrant metabolism, trafficking or turnover of Aβ including, but not limited to, AD.

### SUMMARY OF THE INVENTION

The instant application discloses human homologs of several Drosophila genes as suitable targets for the development of new therapeutics to treat, prevent or ameliorate neurodegenerative conditions including, but not limited to, AD. Thus, in one aspect the invention relates to a method to identify modulators useful to treat, prevent or ameliorate said conditions comprising:
(a) assaying for the ability of a candidate modulator, in vitro or in vivo, to modulate the biochemical function of a protein selected from the group consisting of those disclosed in Table 3 or 3A and/or modulate the expression of a gene encoding said protein; and which can further include
(b) assaying for the ability of an identified modulator to reverse the pathological effects observed in animal models of said neurodegenerative conditions and/or in clinical studies with subjects with said conditions.

In another aspect, the invention relates to a method to treat, prevent or ameliorate neurodegenerative conditions including, but not limited to, AD, comprising administering to a subject in need thereof an effective amount of a modulator of a protein selected from the group consisting of those disclosed in Table 3 or 3A, wherein said modulator, e.g., inhibits or enhances the biochemical function of said protein. In a further embodiment, the modulator comprises antibodies to said protein or fragments thereof, wherein said antibodies can inhibit the biochemical function of said protein in said subject.

In another embodiment, the modulator inhibits or enhances the RNA expression of a gene encoding for a protein selected from the group consisting of those disclosed in Table 3 or 3A. In a further embodiment, the modulator comprises any one or more substances selected from the group consisting of antisense oligonucleotides, triple-helix DNA, ribozymes, RNA and DNA aptamers, siRNA and double- or single-stranded RNA, wherein said substances are designed to inhibit RNA expression of gene encoding said protein.

In another aspect, the invention relates to a method to treat, prevent or ameliorate neurodegenerative conditions including, but not limited to, AD, comprising administering to a subject in need thereof a pharmaceutical composition comprising an effective amount of a modulator of a protein selected from the group consisting of those disclosed in Table 3 or 3A. In various embodiments, said pharmaceutical composition comprises antibodies to said protein or fragments thereof, wherein said antibodies can inhibit the biochemical function of said protein in said subject and/or any one or more substances selected from the group consisting of antisense oligonucleotides, triple-helix DNA, ribozymes, RNA and DNA aptamers, siRNA and double- or single-stranded RNA, wherein said substances are designed to inhibit RNA expression of gene encoding said protein. It is contemplated herein that one or more modulators of one or more of said proteins may be administered concurrently.

In another aspect, the invention relates to a pharmaceutical composition comprising a modulator to a protein selected from the group consisting of those disclosed in Table 3 or 3A in an amount effective to treat, prevent or ameliorate a neurodegenerative condition including, but not limited to, AD, in a subject in need thereof. In one embodiment, said modulator may, e.g., inhibit or enhance the biochemical functions of said protein. In a further embodiment, said modulator comprises antibodies to said protein or fragments thereof, wherein said antibodies can, e.g., inhibit the biochemical functions of said protein.

In a further embodiment, said pharmaceutical composition comprises a modulator which may, e.g., inhibit or enhance RNA expression of gene encoding said protein. In a further embodiment, said modulator comprises any one or more substances selected from the group consisting of antisense oligonucleotides, triple-helix DNA, ribozymes, RNA or DNA aptamers, siRNA or double- or single-stranded RNA directed to a nucleic acid sequence of said protein, wherein said substances are designed to inhibit RNA expression of gene encoding said protein.

In another aspect, the invention relates to a method to diagnose subjects suffering from a neurodegenerative condition who may be suitable candidates for treatment with modulators to a protein selected from the group consisting of those disclosed in Table 3 or 3A, comprising detecting levels of any one or more of said proteins in a biological sample from said subject wherein subjects with altered levels compared to controls would be suitable candidates for modulator treatment.

In another aspect, the invention relates to a method to diagnose subjects suffering from a neurodegenerative condition including, but not limited to, AD, who may be suitable candidates for treatment with modulators to a protein selected from the group consisting of those disclosed in Table 3 or 3A, comprising assaying messenger RNA (mRNA) levels of any one or more of said protein in a biological sample from said subject, wherein subjects with altered levels compared to controls would be suitable candidates for modulator treatment.

In yet another aspect, there is provided a method to treat, prevent or ameliorate neurodegenerative conditions including, but not limited to, AD, comprising:
(a) assaying for mRNA and/or protein levels of a protein selected from the group consisting of those disclosed in Table 3 or 3A in a subject; and
(b) administering to a subject with altered levels of mRNA and/or protein levels compared to controls a modulator to said protein in an amount sufficient to treat, prevent or ameliorate said condition.

In particular embodiments, said modulator inhibits or enhances the biochemical function of said protein or RNA expression of gene encoding said protein.

In yet another aspect of the present invention, there are provided assay methods and diagnostic kits comprising:
(a) the components necessary to detect mRNA levels or protein levels of any one or more proteins selected from the group consisting of those disclosed in Table 3 or 3A in a biological sample, said kit comprising, e.g., polynucleotides encoding any one or more proteins selected from the group consisting of those disclosed in Table 3 or 3A; and
(b) nucleotide sequences complementary to said protein;
(c) any one or more of said proteins, or fragments thereof of antibodies that bind to any one or more of said proteins, or to fragments thereof.

In a preferred embodiment, such kits also comprise instructions detailing the procedures by which the kit components are to be used.

The present invention also pertains to the use of a modulator to a protein selected from the group consisting of those disclosed in Table 3 or 3A, in the manufacture of a medicament for the treatment, prevention or amelioration of neurodegenerative conditions including, but not limited to, AD. In one embodiment, said modulator comprises any one or more substances selected from the group consisting of antisense oligonucleotides, triple-helix DNA, ribozymes, RNA aptamer, siRNA and double- or single-stranded RNA, wherein said substances are designed to inhibit gene expression of said protein. In yet a further embodiment, said modulator comprises one or more antibodies to said protein or fragments thereof, wherein said antibodies or fragments thereof can, e.g., inhibit the biochemical function of said protein.

The invention also pertains to a modulator to a protein selected from the group consisting of those disclosed in Table 3 or 3A for use as a pharmaceutical. In one embodiment, said modulator comprises any one or more substances selected from the group consisting of antisense oligonucleotides, triple-helix DNA, ribozymes, RNA aptamer, siRNA and double- or single-stranded RNA, wherein said substances are designed to inhibit gene expression of said protein. In yet a further embodiment, said modulator comprises one or more antibodies to said protein or fragments thereof, wherein said antibodies or fragments thereof can, e.g., inhibit the biochemical functions of said protein.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### DESCRIPTION OF THE FIGURES

FIG. 1 depicts the typical parental crosses used for the EP based genetic screen to find modifiers of Aβ-induced rough-eye phenotype. FM7, MKRS and TM6 are commonly used balancers for X, 2^{nd} and 3^{rd} chromosomes, respectively.

FIG. 2 depicts the sequence of Aβ42 with the preproenkephalin signal sequence. Italicized letters depict amino acids. Non-underlined letters depict the pre-proenkephaline signal sequence and underlined letters depict the sequence of Aβ42.

### DETAILED DESCRIPTION OF THE INVENTION

All patent applications, patents and literature references cited herein are hereby incorporated by reference in their entirety.

Abbreviations used in the following description include:

| | |
|---|---|
| BBS | BES buffered solution |
| DsRed | Discosoma Red Fluorescent Protein |
| ELISA | Enzyme linked immunosorbent assay |
| IDE | insulin degrading enzyme |
| min. | minutes |
| MTGal4 | Metallothionin Gal4 driver |
| nGFP | Nuclear Green Fluorescent Protein |
| PBS | Phosphate buffered saline |
| RT | Room temperature |

In practicing the present invention, many conventional techniques in molecular biology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Edition, Cold Spring Harbor Laboratory Press, Cold Spring Harbor, N.Y. (1989); DNA Cloning: A Practical Approach, Vols. I and II, Glover, Ed. (1985); Oligonucleotide Synthesis, Gait, Ed. (1984); Nucleic Acid Hybridization, Hames and Higgins, Eds. (1985); Transcription and Translation, Hames and Higgins, Eds. (1984); Animal Cell Culture, Freshney, Ed. (1986); Immobilized Cells and Enzymes, IRL Press (1986); Perbal, A Practical Guide to Molecular Cloning; the series, Meth Enzymol, Academic Press, Inc. (1984); Gene Transfer Vectors for Mammalian Cells, Miller and Calos, Eds., Cold Spring Harbor Laboratory Press, NY (1987); and Methods in Enzymology, Vols. 154 and 155, Wu and Grossman, and Wu, Eds., respectively (1987). Well-known Drosophila-molecular genetics techniques can be found, e.g., in Drosophila, A Practical Approach, Robert, Ed., IRL Press, Washington D.C. (1986).

Descriptions of flystocks can be found in the Flybase database at http://flybase.bio.indiana.edu.

Stock centers referred to herein include Bloomington and Szeged stock centers which are located at Bloomington, Ind. and Szeged, Hungary, respectively.

As used herein and in the appended claims, the singular forms “a”, “an” and “the” include plural reference unless the context clearly dictates otherwise. Thus, e.g., reference to “the antibody” is a reference to one or more antibodies and equivalents thereof known to those skilled in the art, and so forth.

“Nucleic acid sequence”, as used herein, refers to an oligonucleotide, nucleotide or polynucleotide, and fragments or portions thereof, and to DNA or RNA of genomic or synthetic origin that may be single- or double-stranded, and represent the sense or antisense strand.

The term “degenerate nucleotide sequence” refers to a sequence of nucleotides that includes one or more degenerate codons (as compared to a reference polynucleotide molecule that encodes a polypeptide). Degenerate codons contain different triplets of nucleotides, but encode the same amino acid residue, i.e., GAU and GAC triplets each encode Asp. Some polynucleotides encompassed by a degenerate sequence may have some variant amino acids, but one of ordinary skill in the art can easily identify such variant sequences by reference to the amino acid sequences encoding the proteins disclosed in Table 3 or 3A. Variants of the disclosed proteins in Table 3 or 3A can be generated through DNA shuffling as disclosed by Stemmer, Nature, Vol. 370, No. 6488, pp. 389-391 (1994); and Stemmer, Proc Natl Acad Sci USA, Vol. 91, No. 22, pp. 10747-10751 (1994). Variant sequences can be readily tested for functionality as described herein.

“Allelic variant” refers to any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

Allelic variants can be cloned by probing cDNA or genomic libraries from different individuals according to standard procedures. Allelic variants of the DNA sequences encoding proteins disclosed in Table 3 or 3A and variants thereof, including those containing silent mutations and those in which mutations result in amino acid sequence changes, are within the scope of the present invention.

“Splice variant” refers to alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term “splice variant” is also used herein to denote a protein encoded by a splice variant of an mRNA transcribed from a gene.

The term “antisense”, as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. The designation “negative” is sometimes used in reference to the antisense strand, and “positive” is sometimes used in reference to the sense strand.

“cDNA” refers to DNA that is complementary to a portion of mRNA sequence and is generally synthesized from an mRNA preparation using reverse transcriptase.

As contemplated herein, antisense oligonucleotides, triple-helix DNA, RNA aptamers, ribozymes, siRNA and double- or single-stranded RNA are directed to a nucleic acid sequence such that the nucleotide sequence chosen will produce gene-specific inhibition of gene expression. For example, knowledge of a nucleotide sequence may be used to design an antisense molecule which gives strongest hybridization to the mRNA. Similarly, ribozymes can be synthesized to recognize specific nucleotide sequences of a gene and cleave it. See Cech, JAMA, Vol. 260, No. 20, pp. 3030-3034 (1988). Techniques for the design of such molecules for use in targeted inhibition of gene expression is well-known to one of skill in the art.

The individual proteins/polypeptides referred to herein include any and all forms of these proteins including, but not limited to, partial forms, isoforms, variants, precursor forms, the full-length protein, fusion proteins containing the sequence or fragments of any of the above, from human or any other species. Protein homologs or orthologs which would be apparent to one of skill in the art are included in this definition. These proteins/polypeptides may further comprise variants wherein the resulting polypeptide will be at least 80-90% or in other embodiments, at least 95%, 96%, 97%, 98% or 99% identical to the corresponding region of a sequence selected from Table 3 or 3A. Percent sequence identity is determined by conventional methods. See, e.g., Altschul and Erickson, Bull Math Biol, Vol. 48, Nos. 5-6, pp. 603-616 (1986); and Henikoff and Henikoff, Proc Natl Acad Sci USA, Vol. 89, No. 22, pp. 10915-10919 (1992). Briefly, two amino acid sequences are aligned to optimize the alignment scores using a gap opening penalty of 10, a gap extension penalty of 1, and the “BLOSUM62” scoring matrix of Henikoff and Henikoff. The percent identity is then calculated as:

1[(total number of identical matches) length of the longer sequence plus the number of gaps introduced into the longer sequence in order to align the two sequences]×100

It is also contemplated that the term refers to proteins isolated from naturally-occurring sources of any species, such as genomic DNA libraries, as well as genetically-engineered host cells comprising expression systems, or produced by chemical synthesis using, for instance, automated peptide synthesizers or a combination of such methods. Means for isolating and preparing such polypeptides are well-understood in the art.

The term “sample”, as used herein, is used in its broadest sense. A biological sample from a subject may comprise blood, urine, brain tissue, primary cell lines, immortalized cell lines or other biological material with which protein activity or gene expression may be assayed. A biological sample may include, e.g., blood, tumors or other specimens from which total RNA may be purified for gene expression profiling using, e.g., conventional glass chip microarray technologies, such as Affymetrix chips, RT-PCR or other conventional methods.

As used herein, the term “antibody” refers to intact molecules, as well as fragments thereof, such as Fa, F(ab′)₂ and Fv, which are capable of binding the epitopic determinant. Antibodies that bind specific polypeptides can be prepared using intact polypeptides or fragments containing small peptides of interest as the immunizing antigen. The polypeptides or peptides used to immunize an animal can be derived from the translation of RNA or synthesized chemically, and can be conjugated to a carrier protein. Commonly used carriers that are chemically coupled to peptides include bovine serum albumin and thyroglobulin. The coupled peptide is then used to immunize an animal, e.g., a mouse, goat, chicken, rat or a rabbit.

The term “humanized antibody”, as used herein, refers to antibody molecules in which amino acids have been replaced in the non-antigen binding regions in order to more closely resemble a human antibody, while still retaining the original binding ability.

A “therapeutically effective amount” is the amount of drug sufficient to treat, prevent or ameliorate a neurodegenerative condition, specifically a condition involving the aberrant metabolism, trafficking or turnover of Aβ including, but not limited to, AD.

A “transgenic” organism as used herein refers to an organism that has had extra genetic material inserted into its genome. As used herein, a “transgenic fly” includes embryonic, larval and adult forms of Drosophila that contain a DNA sequence from the same or another organism randomly inserted into their genome. Although Drosophila melanogaster is preferred, it is contemplated that any fly of the genus Drosophila may be used in the present invention.

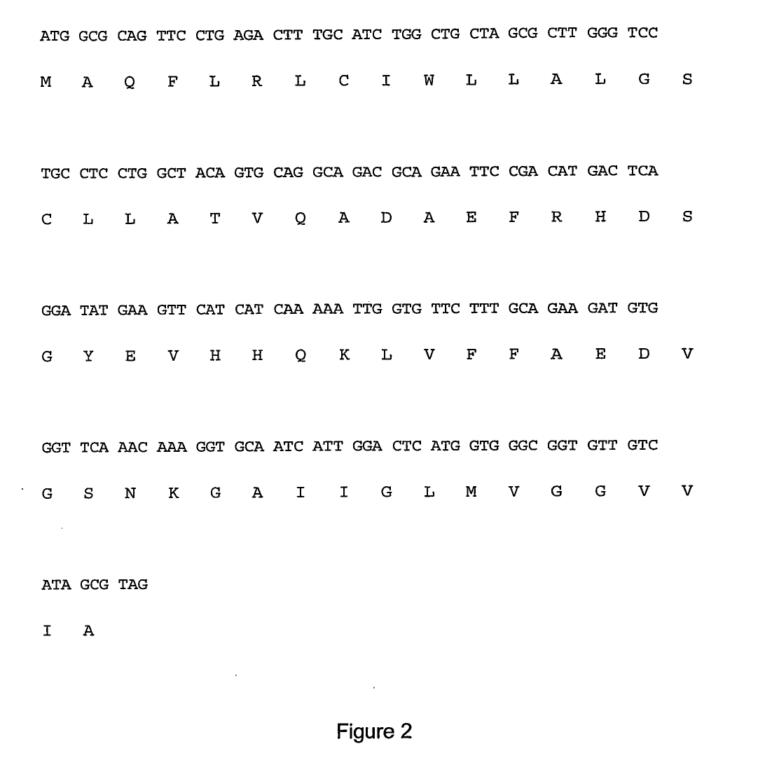

As used herein, the term “Aβ” refers to beta- (β-)amyloid peptide which is a short (42 amino acid) peptide produced by proteolytic cleavage of APP by β and gamma (γ) secretases. It is the primary component of amyloid depositions, the hallmark of AD and the cause of neuronal cell death and degeneration. Aβ42 is provided herein as SEQ ID NO: 1 (see FIG. 2).

As the term is used herein, the “rough-eye” phenotype is characterized by disorganization of ommatidia and inter-ommatidial bristles and can be caused by degeneration of neuronal cells.

As used herein, “ectopic” expression of the transgene refers to expression of the transgene in a tissue or cell or at a specific developmental stage where it is not normally expressed.

As used herein, “phenotype” refers to the observable physical or biochemical characteristics of an organism as determined by both genetic makeup and environmental influences.

As used herein, “neurodegenerative conditions” include those conditions associated with progressive deterioration of the nervous system, caused, e.g., by errors in the regulation of the APP pathway, specifically, conditions involving, e.g., the aberrant metabolism, trafficking or turnover of Aβ including, but not limited to, AD.

The term “transcription factor” refers to any protein required to initiate or regulate transcription in eukaryotes. For example, the eye-specific promoter GMR is a binding site for the eye-specific transcription factor, GLASS. See Moses and Rubin, Genes Dev, Vol. 5, No. 4, pp. 583-593 (1991).

“UAS region”, as used herein, refers to an UAS recognized by the Gal4 transcriptional activator.

As used herein, a “control fly” refers to a larva or fly that is of the same genotype as larvae or flies used in the methods of the present invention except that the control larva or fly does not carry the mutation being tested for modification of phenotype.

As used herein, a “transformation vector” is a modified transposable element used with the transposable element technique to mediate integration of a piece of DNA in the genome of the organism and is familiar to one of skill in the art.

As used herein, “elevated transcription of mRNA” refers to a greater amount of mRNA transcribed from the natural endogenous gene encoding a protein, e.g., a human protein set forth in Table 3 or 3A, compared to control levels. Elevated mRNA levels of a protein, e.g., a human protein disclosed on Table 3 or 3A, may be present in a tissue or cell of an individual suffering from a neurodegenerative condition compared to levels in a subject not suffering from said condition. In particular, levels in a subject suffering from said condition may be at least about twice, preferably at least about five times, more preferably at least about 10 times, most preferably at least about 100 times the amount of mRNA found in corresponding tissues in humans who do not suffer from said condition. Such elevated level of mRNA may eventually lead to increased levels of protein translated from such mRNA in an individual suffering from said condition as compared to levels in a healthy individual.

As used herein, a “Drosophila transformation vector” is a DNA plasmid that contains transposable element sequences and can mediate integration of a piece of DNA in the genome of the organism. This technology is familiar to one of skill in the art.

Methods of obtaining transgenic organisms, including transgenic Drosophila, are well-known to one skilled in the art. For example, a commonly used reference for P-element mediated transformation is Spradling, Drosophila: A practical approach, Roberts, Ed., pp. 175-197, IRL Press, Oxford, UK (1986). The EP element technology refers to a binary system, utilizing the yeast Gal4 transcriptional activator, that is used to ectopically regulate the transcription of endogenous Drosophila genes. This technology is described in Brand and Perrimon, Development, Vol. 118, No. 2, pp. 401-415 (1993); and Rorth (1998), supra.

A “host cell”, as used herein, refers to a prokaryotic or eukaryotic cell that contains heterologous DNA that has been introduced into the cell by any means, e.g., electroporation, calcium phosphate precipitation, microinjection, transformation, viral infection and the like.

“Heterologous”, as used herein, means “of different natural origin” or represents a non-natural state. For example, if a host cell is transformed with a DNA or gene derived from another organism, particularly from another species, that gene is heterologous with respect to that host cell and also with respect to descendants of the host cell which carry that gene. Similarly, heterologous refers to a nucleotide sequence derived from and inserted into the same natural, original cell type, but which is present in a non-natural state, e.g., a different copy number, or under the control of different regulatory elements.

A “vector” molecule is a nucleic acid molecule into which heterologous nucleic acid may be inserted which can then be introduced into an appropriate host cell. Vectors preferably have one or more origin of replication, and one or more site into which the recombinant DNA can be inserted. Vectors often have convenient means by which cells with vectors can be selected from those without, e.g., they encode drug resistance genes. Common vectors include plasmids, viral genomes, and (primarily in yeast and bacteria) “artificial chromosomes”.

“Plasmids” generally are designated herein by a lower case p preceded and/or followed by capital letters and/or numbers, in accordance with standard naming conventions that are familiar to those of skill in the art. Starting plasmids disclosed herein are either commercially-available, publicly-available on an unrestricted basis, or can be constructed from available plasmids by routine application of well-known, published procedures. Many plasmids and other cloning and expression vectors that can be used in accordance with the present invention are well-known and readily-available to those of skill in the art. Moreover, those of skill, readily may construct any number of other plasmids suitable for use in the invention. The properties, construction and use of such plasmids, as well as other vectors, in the present invention will be readily apparent to those of skill from the present disclosure.

The term “isolated” means that the material is removed from its original environment, e.g., the natural environment, if it is naturally-occurring. For example, a naturally-occurring polynucleotide or polypeptide present in a living animal is not isolated, but the same polynucleotide or polypeptide, separated from some or all of the co-existing materials in the natural system, is isolated, even if subsequently reintroduced into the natural system. Such polynucleotides could be part of a vector and/or such polynucleotides or polypeptides could be part of a composition, and still be isolated in that such vector or composition is not part of its natural environment.

As used herein, the term “transcriptional control sequence” or “expression control sequence” refers to DNA sequences, such as initiator sequences, enhancer sequences and promoter sequences, which induce, repress or otherwise control the transcription of a protein encoding nucleic acid sequences to which they are operably-linked. They may be tissue specific and developmental-stage specific.

A “human transcriptional control sequence” is a transcriptional control sequence normally found associated with the human gene encoding a polypeptide set forth in Table 3 or 3A of the present invention as it is found in the respective human chromosome.

A “non-human transcriptional control sequence” is any transcriptional control sequence not found in the human genome.

The term “polypeptide” is used, interchangeably herein, with the terms “polypeptides” and “protein(s)”.

A chemical derivative of a protein set forth in Table 3 or 3A of the invention is a polypeptide that contains additional chemical moieties not normally a part of the molecule. Such moieties may improve the molecule's solubility, absorption, biological half-life, etc. The moieties may alternatively decrease the toxicity of the molecule, eliminate or attenuate any undesirable side effect of the molecule, etc. Moieties capable of mediating such effects are disclosed, e.g., in Remington's Pharmaceutical Sciences, 16^{th} Edition, Mack Publishing Co., Easton, Pa. (1980).

The ability of a substance to “modulate” a protein set forth in Table 3 or 3A or a variant thereof, i.e., “a modulator of a protein selected from the group consisting of those disclosed in Table 3 or 3A” includes, but is not limited to, the ability of a substance to inhibit or enhance the activity of said protein and/or variant thereof and/or inhibit or enhance the RNA expression of gene encoding said protein or variant. Such modulation could also involve affecting the ability of other proteins to interact with said protein, e.g., related regulatory proteins or proteins that are modified by said protein.

The term “agonist”, as used herein, refers to a molecule, i.e., modulator, which, directly or indirectly, may modulate a polypeptide, e.g., a polypeptide set forth in Table 3 or 3A or a variant thereof, and which increases the biological activity of said polypeptide. Agonists may include proteins, nucleic acids, carbohydrates or other molecules. A modulator that enhances gene transcription or the biochemical function of a protein is something that increases transcription or stimulates the biochemical properties or activity of said protein, respectively.

The terms “antagonist” or “inhibitor” as used herein, refer to a molecule, i.e., modulator, which directly or indirectly may modulate a polypeptide or variant thereof, e.g., a polypeptide set forth in Table 3 or 3A, which blocks or inhibits the biological activity of said polypeptide. Antagonists and inhibitors may include proteins, nucleic acids, carbohydrates or other molecules. A modulator that inhibits gene expression or the biochemical function of a protein is something that reduces gene expression or biological activity of said protein, respectively.

As generally referred to herein, a “protein or gene selected from the group consisting of those disclosed in Table 3 or 3A” refers to the human form of the protein or gene. It is recognized, that polypeptides (or nucleic acids which encode those polypeptides) containing less than the described levels of sequence identity to proteins in Table 3 or 3A and arising as splice or allelic variants or that are modified by minor deletions, by conservative amino acid substitutions, by substitution of degenerate codons or the like, also are encompassed within the scope of the present invention. A variety of known algorithms are known in the art and have been disclosed publicly, and a variety of commercially-available software for conducting homology-based similarity searches are available and can be used to identify variants of proteins disclosed herein. Examples of such software includes, but are not limited to, FASTA (GCG Wisconsin Package), Bic_SW (Compugen Bioccelerator), BLASTN2, BLASTP2, BLASTD2 (NCBI) and Motifs (GCG). Suitable software programs are described, e.g., in Guide to Human Genome Computing, 2^{nd} edition, Bishop, Ed., Academic Press, San Diego, Calif. (1998); and The Internet and the New Biology: Tools for Genomic and Molecular Research, American Society for Microbiology, Peruski, Jr. and Harwood Peruski, Eds., Washington, D.C. (1997).

“In vivo models of a neurodegenerative condition, specifically conditions involving the aberrant metabolism, trafficking or turnover of Aβ” include those in vivo models of neurodegenerative diseases, such as AD, familiar to those of skill in the art. Such in vivo models include, e.g., the mouse model of AD disclosed in WO 94/00569. In addition, numerous cell lines may be used as in vitro models of AD and are familiar to one of skill in the art including, e.g., the cell lines. See Xia et al., PNAS USA, Vol. 94, No. 15, pp. 8208-8213 (1997).

The genes of the present invention were identified using a transgenic fly, Drosophila melanogaster, whose genome comprises a DNA sequence encoding Aβ. Conventional expression control systems may be used to achieve ectopic expression of proteins of interest, including the Aβ peptide. Such expression may result in the disturbance of biochemical pathways and the generation of altered phenotypes. One such expression control system involves direct fusion of the DNA sequence to expression control sequences of tissue-specifically-expressed genes, such as promoters or enhancers. A tissue-specific expression control system that may be used is the binary Gal4-transcriptional activation system. See Brand and Perrimon (1993), supra.

The Gal4 system uses the yeast transcriptional activator Gal4, to drive the expression of a gene of interest in a tissue-specific manner. The Gal4 gene has been randomly inserted into the fly genome, using a conventional transformation system, so that it has come under the control of genomic enhancers that drive expression in a temporal and tissue-specific manner. Individual strains of flies have been established, called “drivers”, that carry those insertions. See Brand and Perrimon (1993), supra.

In the Gal4 system, a gene of interest is cloned into a transformation vector, so that its transcription is under the control of the UAS and the Gal4-responsive element. When a fly strain that carries the UAS gene of interest sequence is crossed to a fly strain that expresses the Gal4 gene under the control of a tissue-specific enhancer, the gene will be expressed in a tissue-specific pattern.

In order to generate phenotypes that are easily visible in adult tissues and can thus be used in genetic screens, Gal4 “drivers” that drive expression in later stages of the fly development may be used in the present invention. Using these drivers, expression would result in possible defects in the wings, the eyes, the legs, different sensory organs and the brain. These “drivers” include, e.g., apterous-Gal4 (wings), elav-Gal4 (CNS), sevenless-Gal4, eyGal4 and pGMR-Gal4 (eyes). Descriptions of the Gal4 lines and notes about their specific expression patterns is available in Flybase (http:/flybase.bio.indiana.edu).

Various DNA constructs may be used to generate a transgenic Drosophila melanogaster. For example, the construct may contain the Aβ-sequence cloned into the pUAST vector [see Brand and Perrimon (1993), supra] which places the UAS up-stream of the transcribed region. Insertion of these constructs into the fly genome may occur through P-element recombination, Hobo element recombination [see Blackman et al., EMBO J, Vol. 8, No. 1, pp. 211-217 (1989)], homologous recombination [see Rong and Golic, Science, Vol. 288, No. 5473, pp. 2013-2018 (2000)] or other standard techniques known to one of skill in the art.

As discussed above, an ectopically-expressed gene may result in an altered phenotype by disruption of a particular biochemical pathway. Mutations in genes acting in the same biochemical pathway are expected to cause modification of the altered phenotype. Thus, the transgenic fly carrying the Aβ42-sequence is used to identify genes involved in the development and/or progression of neurodegenerative conditions, e.g., conditions involving the aberrant metabolism, trafficking or turnover of Aβ, such as AD, by crossing this transgenic fly with a fly containing a mutation in a known or predicted gene; and screening progeny of the crosses for flies that display quantitative or qualitative modification of the “rough-eye” phenotype of the Aβ42 transgenic fly, as compared to controls.

This system is extremely beneficial for the elucidation of the function of Aβ gene products, as well as the identification of other genes that directly or indirectly interact with them. Mutations that can be screened include, but are not limited to, loss-of-function alleles of known genes, deletion strains, “enhancer-trap” strains generated by the P-element and gain-of-function mutations generated by random insertions into the Drosophila genome of a Gal4-inducible construct that can activate the ectopic expression of genes in the vicinity of its insertion. In this way, genes involved in the development and/or progression of neurodegenerative conditions can be identified and these genes and polypeptides encoded by these genes can then serve as targets for the development of therapeutics to treat such conditions.

Nucleic acid molecules of the human homologs of the target polypeptides disclosed herein may act as target gene antisense molecules, useful, e.g., in target gene regulation and/or as antisense primers in amplification reactions of target gene nucleic acid sequences. Further, such sequences may be used as part of ribozyme and/or triple-helix sequences or as targets for siRNA or double- or single-stranded RNA, which may be employed for gene regulation. Still further, such molecules may be used as components of diagnostic kits as disclosed herein.

In cases where an identified gene is the normal or wild type gene, this gene may be used to isolate mutant alleles of the gene. Such isolation is preferable in processes and disorders which are known or suspected to have a genetic basis. Mutant alleles may be isolated from individuals either known or suspected to have a genotype which contributes to disease symptoms related to neurodegenerative conditions including, but not limited to, AD. Mutant alleles and mutant allele products may then be utilized in the diagnostic assay systems described herein.

A cDNA of the mutant gene may be isolated, e.g., by using PCR, a technique which is well-known to those of skill in the art. In this case, the first cDNA strand may be synthesized by hybridizing an oligo-dT oligonucleotide to mRNA isolated from tissue known or suspected to be expressed in an individual putatively carrying the mutant allele, and by extending the new strand with reverse transcriptase. The second strand of the complementary (cDNA) is then synthesized using an oligonucleotide that hybridizes specifically to the 5′ end of the normal gene. Using these two primers, the product is then amplified via PCR, cloned into a suitable vector, and subjected to DNA sequence analysis through methods well-known to those of skill in the art. By comparing the DNA sequence of the mutant gene to that of the normal gene, the mutation(s) responsible for the loss or alteration of function of the mutant gene product can be ascertained.

Alternatively, a genomic or cDNA library can be constructed and screened using DNA or RNA, respectively, from a tissue known to or suspected of expressing the gene of interest in an individual suspected of or known to carry the mutant allele. The normal gene or any suitable fragment thereof may then be labeled and used as a probe to identify the corresponding mutant allele in the library. The clone containing this gene may then be purified through methods routinely practiced in the art, and subjected to sequence analysis as described above.

Additionally, an expression library can be constructed utilizing DNA isolated from or cDNA synthesized from a tissue known to or suspected of expressing the gene of interest in an individual suspected of or known to carry the mutant allele. In this manner, gene products made by the putatively mutant tissue may be expressed and screened using standard antibody screening techniques in conjunction with antibodies raised against the normal gene product, as described below. For screening techniques, see, e.g., Antibodies: A Laboratory Manual, Harlow and Lane, Eds., Cold Spring Harbor Press, Cold Spring Harbor, N.Y. (1988). In cases where the mutation results in an expressed gene product with altered function, e.g., as a result of a mis-sense mutation, a polyclonal set of antibodies are likely to cross-react with the mutant gene product. Library clones detected via their reaction with such labeled antibodies can be purified and subjected to sequence analysis as described above.

In another embodiment, nucleic acids comprising a sequence encoding a polypeptide set forth in Table 3 or 3A or a functional-derivative thereof, may be administered to promote normal biological function, e.g., normal Aβ turnover, by way of gene therapy. Gene therapy refers to therapy performed by the administration of a nucleic acid to a subject. In this embodiment of the invention, the nucleic acid produces its encoded protein that mediates a therapeutic effect by, e.g., promoting normal Aβ turnover.

Any of the methods for gene therapy available in the art can be used according to the present invention. Exemplary methods are described below.

In a preferred aspect, the therapeutic comprises a nucleic acid for a Table 3 or 3A polypeptide that is part of an expression vector that expresses a Table 3 or 3A protein, fragment or chimeric protein thereof and variants thereof in a suitable host. In particular, such a nucleic acid has a promoter operably-linked to the Table 3 or 3A protein coding region, said promoter being inducible or constitutive, and, optionally, tissue-specific. In another particular embodiment, a nucleic acid molecule is used in which the Table 3 or 3A protein coding sequences and any other desired sequences are flanked by regions that promote homologous recombination at a desired site in the genome, thus providing for intrachromosomal expression of the Table 3 or 3A nucleic acid. See Koller and Smithies, Proc Natl Acad Sci USA, Vol. 86, No. 22, pp. 8932-8935 (1989); and Zijistra et al., Nature, Vol. 342, No. 6248, pp. 435-438 (1989).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see, e.g., U.S. Pat. No. 4,980,286 and others mentioned infra), or by direct injection of naked DNA, or by use of microparticle bombardment, e.g., a gene gun; Biolistic, Dupont, or coating with lipids or cell-surface receptors or transfecting agents, encapsulation in liposomes, microparticles or microcapsules, or by administering it in linkage to a peptide which is known to enter the nucleus, by administering it in linkage to a ligand subject to receptor-mediated endocytosis (see, e.g., U.S. Pat. Nos. 5,166,320; 5,728,399; 5,874,297 and 6,030,954, all of which are incorporated by reference herein in their entirety), which can be used to target cell types specifically expressing the receptors, etc. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor. See, e.g., PCT Publications WO 92/06180; WO 92/22635; WO 92/20316; WO 93/14188 and WO 93/20221. Alternatively, the nucleic acid can be introduced intracellularly and incorporated within host cell DNA for expression, by homologous recombination. See, e.g., U.S. Pat. Nos. 5,413,923; 5,416,260 and 5,574,205; and Zijistra et al. (1989), supra.

In a specific embodiment, a viral vector that contains a nucleic acid encoding a Table 3 or 3A polypeptide is used. For example, a retroviral vector can be used. See, e.g., U.S. Pat. Nos. 5,219,740; 5,604,090 and 5,834,182. These retroviral vectors have been modified to delete retroviral sequences that are not necessary for packaging of the viral genome and integration into host cell DNA. The nucleic acid for the Table 3 or 3A polypeptide to be used in gene therapy is cloned into the vector, which facilitates delivery of the gene into a patient.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Methods for conducting adenovirus-based gene therapy are described in, e.g., U.S. Pat. Nos. 5,824,544; 5,868,040; 5,871,722; 5,880,102; 5,882,877; 5,885,808; 5,932,210; 5,981,225; 5,994,106; 5,994,132; 5,994,134; 6,001,557 and 6,033,8843, all of which are incorporated by reference herein in their entirety.

Adeno-associated virus (AAV) has also been proposed for use in gene therapy. Methods for producing and utilizing AAV are described, e.g., in U.S. Pat. Nos. 5,173,414; 5,252,479; 5,552,311; 5,658,785; 5,763,416; 5,773,289; 5,843,742; 5,869,040; 5,942,496 and 5,948,675, all of which are incorporated by reference herein in their entirety.

Another approach to gene therapy involves transferring a gene to cells in tissue culture by such methods as electroporation, lipofection, calcium phosphate mediated transfection or viral infection. Usually, the method of transfer includes the transfer of a selectable marker to the cells. The cells are then placed under selection to isolate those cells that have taken up and are expressing the transferred gene. Those cells are then delivered to a patient.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient. Recombinant blood cells, e.g., hematopoietic stem or progenitor cells, are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type and include, but are not limited to, epithelial cells, endothelial cells, keratinocytes, fibroblasts, muscle cells, hepatocytes; blood cells, such as T lymphocytes, B lymphocytes, monocytes, macrophages, neutrophils, eosinophils, megakaryocytes, granulocytes; various stem or progenitor cells, in particular, hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment, in which recombinant cells are used in gene therapy, the nucleic acid of a polypeptide set forth in Table 3 or 3A is introduced into the cells such that it is expressible by the cells or their progeny, and the recombinant cells are then administered in vivo for therapeutic effect. In a specific embodiment, stem or progenitor cells are used. Any stem cells and/or progenitor cells that can be isolated and maintained in vitro can potentially be used in accordance with this embodiment of the present invention. Such stem cells include, but are not limited to, hematopoietic stem cells (HSC), stem cells of epithelial tissues such as the skin and the lining of the gut, embryonic heart muscle cells, liver stem cells (see, e.g., WO 94/08598) and neural stem cells. See Stemple and Anderson, Cell, Vol. 71, No. 6, pp. 973-985 (1992).

Epithelial stem cells (ESCs) or keratinocytes can be obtained from tissues, such as the skin and the lining of the gut by known procedures. See Rheinwald, Methods Cell Biol, Vol. 21A, pp. 229-254 (1980). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture. See Pittelkow and Scott, Mayo Clin Proc, Vol. 61, No. 10, pp. 771-777 (1986). If the ESCs are provided by a donor, a method for suppression of host versus graft reactivity, e.g., irradiation, drug or antibody administration to promote moderate immunosuppression, can also be used.

With respect to HSCs, any technique which provides for the isolation, propagation and maintenance in vitro of HSCs can be used in this embodiment of the invention. Techniques by which this may be accomplished include:
(a) the isolation and establishment of HSC cultures from bone marrow cells isolated from the future host or a donor; or
(b) the use of previously established long-term HSC cultures, which may be allogeneic or xenogeneic.

Non-autologous HSC are used preferably in conjunction with a method of suppressing transplantation immune reactions of the future host/patient. In a particular embodiment of the present invention, human bone marrow cells can be obtained from the posterior iliac crest by needle aspiration. See, e.g., Kodo, Gale and Saxon, J Clin Invest, Vol. 73, No. 5, pp. 1377-1384 (1984). In a preferred embodiment of the present invention, the HSCs can be made highly enriched or in substantially pure form. This enrichment can be accomplished before, during or after long-term culturing, and can be done by any techniques known in the art. Long-term cultures of bone marrow cells can be established and maintained by using, e.g., modified Dexter cell culture techniques [see Dexter et al., J Cell Physiol, Vol. 91, No. 3, pp. 335-344 (1977)] or Witlock-Witte culture techniques. See Witlock and Witte, Proc Natl Acad Sci USA, Vol. 79, No. 11, pp. 3608-3612 (1982).

In a specific embodiment, the nucleic acid to be introduced for purposes of gene therapy comprises an inducible promoter operably-linked to the coding region, such that expression of the nucleic acid is controllable by controlling the presence or absence of the appropriate inducer of transcription.

A further embodiment of the present invention relates to a method to treat, prevent or ameliorate neurodegenerative conditions including, but not limited to AD, comprising administering to a subject in need thereof an effective amount of a modulator of a protein selected from the group consisting of those disclosed in Table 3 or 3A and/or variants thereof. In one embodiment, the modulator comprises one or more antibodies to said protein, variant or fragments thereof, wherein said antibodies or fragments thereof can inhibit the biochemical function of said protein or variant in said subject.

Described herein are methods for the production of antibodies capable of specifically recognizing one or more differentially expressed gene epitopes. Such antibodies may include, but are not limited to, polyclonal antibodies, monoclonal antibodies (mAbs), humanized or chimeric antibodies, single-chain antibodies, Fab fragments, F(ab′)₂ fragments, fragments produced by a Fab expression library, anti-idiotypic (anti-id) antibodies and epitope-binding fragments of any of the above. Such antibodies may be used, e.g., in the detection of a target protein in a biological sample, or alternatively, as a method for the inhibition of the biochemical function of the protein. Thus, such antibodies may be utilized as part of disease treatment methods, and/or may be used as part of diagnostic techniques whereby patients may be tested, e.g., for abnormal levels of polypeptides set forth in Table 3 or 3A, or for the presence of abnormal forms of these polypeptides.

For the production of antibodies to the Table 3 or 3A polypeptides or variants thereof, various host animals may be immunized by injection with these polypeptides, or a portion thereof. Such host animals may include but are not limited to rabbits, mice, goats, chickens and rats, to name but a few. Various adjuvants may be used to increase the immunological response, depending on the host species including, but not limited to, Freund's (complete and incomplete); mineral gels, such as aluminum hydroxide; surface active substances, such as lysolecithin, pluronic polyols, polyanions, peptides, oil emulsions, keyhole limpet hemocyanin and dinitrophenol; and potentially useful human adjuvants, such as bacille Calmette-Guerin (BCG) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with a Table 3 or 3A polypeptide, or a portion thereof, supplemented with adjuvants as also described above.

Monoclonal antibodies, which are homogeneous populations of antibodies to a particular antigen, may be obtained by any technique that provides for the production of antibody molecules by continuous cell lines in culture. These include, but are not limited to, the hybridoma technique [see Kohler and Milstein, Nature, Vol. 256, No. 5517, pp. 495-497 (1975) and U.S. Pat. No. 4,376,110]; the human B-cell hybridoma technique [see Kosbor et al., Immunol Today, Vol. 4, pp. 72 (1983) and Cole et al., Proc Natl Acad Sci USA, Vol. 80, pp. 2026-2030 (1983)]; and the EBV-hybridoma technique. See Cole et al., Monoclonal Antibodies And Cancer Therapy, Alan R. Liss, Inc., pp. 77-969 (1985). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” [see Morrison et al., Proc Natl Acad Sci USA, Vol. 81, No. 21, pp. 6851-6855 (1984); Neuberger, Williams and Fox, Nature, Vol. 312, No. 5995, pp. 604-608 (1984); Takeda et al., Nature, Vol. 314, No. 6010, pp. 452-454 (1985)] by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single-chain antibodies [U.S. Pat. No. 4,946,778; Bird, Science, Vol. 242, pp. 423-426 (1988); Huston et al., Proc Natl Acad Sci USA, Vol. 85, No. 16, pp. 5879-5883 (1988); and Ward et al., Nature, Vol. 334, pp. 544-546 (1989)] can be adapted to produce differentially-expressed gene, single-chain antibodies. Single-chain antibodies are formed by linking the heavy- and light-chain fragments of the Fv region via an amino acid bridge, resulting in a single-chain polypeptide.

Most preferably, techniques useful for the production of “humanized antibodies” can be adapted to produce antibodies to the polypeptides, fragments, derivatives, and functional equivalents disclosed herein. Such techniques are disclosed in U.S. Pat. Nos. 5,932,448; 5,693,762; 5,693,761; 5,585,089; 5,530,101; 5,910,771; 5,569,825; 5,625,126; 5,633,425; 5,789,650; 5,545,580; 5,661,016 and 5,770,429, the disclosures of all of which are incorporated by reference herein in their entirety.

Antibody fragments that recognize specific epitopes may be generated by known techniques. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed [see Huse et al., Science, Vol. 246, No. 4935, pp. 1275-1281 (1989)] to allow rapid and easy identification of monoclonal Fab fragments with the desired specificity.

As contemplated herein, an antibody of the present invention can be preferably used in a diagnostic kit for detecting levels of a protein disclosed in Table 3 or 3A or antigenic variants thereof in a biological sample, as well as in a method to diagnose subjects suffering from neurodegenerative conditions who may be suitable candidates for treatment with modulators to a protein selected from the group consisting of those disclosed in Table 3 or 3A. Preferably, said detecting step comprises contacting said appropriate tissue cell, e.g., biological sample, with an antibody which specifically binds to a Table 3 or 3A polypeptide, fragment or variants thereof and detecting specific binding of said antibody with a polypeptide in said appropriate tissue, cell or sample wherein detection of specific binding to a polypeptide indicates the presence of a polypeptide set forth in Table 3 or 3A or a fragment thereof.

Particularly preferred, for ease of detection, is the sandwich assay, of which a number of variations exist, all of which are intended to be encompassed by the present invention. For example, in a typical forward assay, unlabeled antibody is immobilized on a solid substrate and the sample to be tested brought into contact with the bound molecule. After a suitable period of incubation, for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well-known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for a Table 3 or 3A polypeptide, fragment or variants thereof.

The most commonly used reporter molecules in this type of assay are either enzymes, fluorophore- or radionuclide-containing molecules. In the case of an enzyme immunoassay, an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, β-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of Table 3 or 3A polypeptide or variant which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually-detectable with a light microscope. Immunofluorescence and EIA techniques are both very well-established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

The pharmaceutical compositions of the present invention may also comprise substances that inhibit the expression of a protein disclosed in Table 3 or 3A or variants thereof at the nucleic acid level. Such molecules include ribozymes, antisense oligonucleotides, triple-helix DNA, RNA aptamers, siRNA and/or double- or single-stranded RNA directed to an appropriate nucleotide sequence of nucleic acid encoding such a protein. These inhibitory molecules may be created using conventional techniques by one of skill in the art without undue burden or experimentation. For example, modifications, e.g., inhibition, of gene expression can be obtained by designing antisense molecules, DNA or RNA, to the control regions of the genes encoding the polypeptides discussed herein, i.e., to promoters, enhancers and introns. For example, oligonucleotides derived from the transcription initiation site, e.g., between positions −10 and +10 from the start site may be used. Notwithstanding, all regions of the gene may be used to design an antisense molecule in order to create those which gives strongest hybridization to the mRNA and such suitable antisense oligonucleotides may be produced and identified by standard assay procedures familiar to one of skill in the art.

Similarly, inhibition of gene expression may be achieved using “triple-helix” base-pairing methodology. Triple helix pairing is useful because it causes inhibition of the ability of the double-helix to open sufficiently for the binding of polymerases, transcription factors or regulatory molecules. Recent therapeutic advances using triplex-DNA have been described in the literature. See Gee et al., Molecular and Immunologic Approaches, Huber and Carr, Eds., Futura Publishing Co., Mt. Kisco, N.Y. (1994). These molecules may also be designed to block translation of mRNA by preventing the transcript from binding to ribosomes.

Ribozymes, enzymatic RNA molecules, may also be used to inhibit gene expression by catalyzing the specific cleavage of RNA. The mechanism of ribozyme action involves sequence-specific hybridization of the ribozyme molecule to complementary target RNA, followed by endonucleolytic cleavage. Examples which may be used include engineered “hammerhead” or “hairpin” motif ribozyme molecules that can be designed to specifically and efficiently catalyze endonucleolytic cleavage of gene sequences. Specific ribozyme cleavage sites within any potential RNA target are initially identified by scanning the target molecule for ribozyme cleavage sites which include the following sequences: GUA, GUU and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Ribozyme methods include exposing a cell to ribozymes or inducing expression in a cell of such small RNA ribozyme molecules. See Grassi and Marini, Ann Med, Vol. 28, No. 6, pp. 499-510 (1996); and Gibson, Cancer Metastasis Rev, Vol. 15, No. 3, pp. 287-299 (1996). Intracellular expression of hammerhead and hairpin ribozymes targeted to mRNA corresponding to at least one of the genes discussed herein can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell. See Cotten and Birnstiel, EMBO J, Vol. 8, No. 12, pp. 3861-3866 (1989). In particular, a ribozyme coding DNA sequence, designed according to conventional, well-known rules and synthesized, e.g., by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter, e.g., a glucocorticoid or a tetracycline response element, is also introduced into this construct so that ribozyme expression can be selectively controlled. For saturating use, a highly and constituently active promoter can be used. tDNA genes, i.e., genes encoding tRNAs, are useful in this application because of their small size, high rate of transcription, and ubiquitous expression in different kinds of tissues.

Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly, the abundance of virtually any RNA species in a cell can be modified or perturbed.

Ribozyme sequences can be modified in essentially the same manner as described for antisense nucleotides, e.g., the ribozyme sequence can comprise a modified base moiety.

RNA aptamers can also be introduced into or expressed in a cell to modify RNA abundance or activity. RNA aptamers are specific RNA ligands for proteins, such as for Tat and Rev RNA [see Good et al., Gene Ther, Vol. 4, No. 1, pp. 45-54 (1997)] that can specifically inhibit their translation.

Gene specific inhibition of gene expression may also be achieved using conventional double- or single-stranded RNA technologies. A description of such technology may be found in WO 99/32619, which is hereby incorporated by reference in its entirety. In addition, siRNA technology has also proven useful as a means to inhibit gene expression. See Cullen, Nat Immunol, Vol. 3, No. 7, pp. 597-599 (2002); and Martinez et al., Cell, Vol. 110, No. 5, pp. 563-574 (2002).

Antisense molecules, triple-helix DNA, RNA aptamers, dsRNA, ssRNA, siRNA and ribozymes of the present invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding the genes of the polypeptides discussed herein. Such DNA sequences may be incorporated into a wide variety of vectors with suitable RNA polymerase promoters, such as T7 or SP6. Alternatively, cDNA constructs that synthesize antisense RNA constitutively or inducibly can be introduced into cell lines, cells or tissues.

Vectors may be introduced into cells or tissues by many available means, and may be used in vivo, in vitro or ex vivo. For ex vivo therapy, vectors may be introduced into stem cells taken from the patient and clonally propagated for autologous transplant back into that same patient. Delivery by transfection and by liposome injections may be achieved using methods that are well-known in the art.

Detection of mRNA levels of proteins disclosed herein may comprise contacting a biological sample or even contacting an isolated RNA or DNA molecule derived from a biological sample with an isolated nucleotide sequence of at least about 20 nucleotides in length that hybridizes under high-stringency conditions, e.g., 0.1×SSPE or SSC, 0.1% SDS, 65° C.) with the isolated nucleotide sequence encoding a polypeptide set forth in Table 3 or 3A. Hybridization conditions may be highly-stringent or less highly-stringent. In instances wherein the nucleic acid molecules are deoxyoligonucleotides (oligos), highly-stringent conditions may refer, e.g., to washing in 6×SSC/0.05% sodium pyrophosphate at 37° C. (for 14-base oligos), 48° C. (for 17-base oligos), 55° C. (for 20-base oligos) and 60° C. (for 23-base oligos). Suitable ranges of such stringency conditions for nucleic acids of varying compositions are described in Krause and Aaronson, Methods Enzymol, Vol. 200, pp. 546-556 (1991) in addition to Maniatis et al., cited above.

In some cases, detection of a mutated form of the gene which is associated with a dysfunction will provide a diagnostic tool that can add to or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acids, in particular mRNA, for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled nucleotide sequences encoding a polypeptide disclosed in Table 3 or 3A or variants thereof. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, e.g., Myers, Larin and Maniatis, Science, Vol. 230, No. 4731, pp. 1242-1246 (1985). Sequence changes at specific locations may also be revealed by nuclease protection assays, such as RNase and S1 protection or the chemical cleavage method. See Cotton et al., Proc Natl Acad Sci USA, Vol. 85, pp. 4397-4401 (1985). In addition, an array of oligonucleotides probes comprising nucleotide sequence encoding the Table 3 or 3A polypeptides, variants or fragments of such nucleotide sequences can be constructed to conduct efficient screening of, e.g., genetic mutations. Array technology methods are well-known and have general applicability and can be used to address a variety of questions in molecular genetics including gene expression, genetic linkage and genetic variability. See, e.g., Chee et al., Science, Vol. 274, No. 5287, pp. 610-613 (1996).

The diagnostic assays offer a process for diagnosing or determining a susceptibility to disease through detection of mutation in the gene of a polypeptide set forth in Table 3 or 3A by the methods described. In addition, such diseases may be diagnosed by methods comprising determining from a sample derived from a subject an abnormally decreased or increased level of polypeptide or mRNA. Decreased or increased expression can be measured at the RNA level using any of the methods well-known in the art for the quantitation of polynucleotides, such as, e.g., nucleic acid amplification, for instance, PCR, RT-PCR, RNase protection, Northern blotting and other hybridization methods. Assay techniques that can be used to determine levels of a protein, such as a polypeptide of the present invention, in a sample derived from a host are well-known to those of skill in the art. Such assay methods include radioimmunoassays, competitive-binding assays, Western Blot analysis and ELISA assays.

Thus in another aspect, the present invention relates to a diagnostic kit for detecting mRNA levels (or protein levels) which comprises:
(a) a polynucleotide of a polypeptide set forth in Table 3 or 3A or a fragment thereof;
(b) a nucleotide sequence complementary to that of Step (a);
(c) a polypeptide of Table 3 or 3A of the present invention encoded by the polynucleotide of Step (a);
(d) an antibody to the polypeptide of Step (c); and
(e) an RNAi sequence complementary to that of Step (a).

It will be appreciated that in any such kit, Step (a), (b), (c), (d) or (e) may comprise a substantial component. Such a kit will be of use in diagnosing a disease or susceptibility to a disease, particularly to a neurodegenerative disease, such as AD.

The nucleotide sequences of the present invention are also valuable for chromosome localization. The sequence is specifically targeted to, and can hybridize with, a particular location on an individual human chromosome. The mapping of relevant sequences to chromosomes is an important first step in correlating those sequences with gene associated disease. Once a sequence has been mapped to a precise chromosomal location, the physical position of the sequence on the chromosome can be correlated with genetic map data. Such data are found in, e.g., V. McKusick, Mendelian Inheritance in Man (available on-line through Johns Hopkins University Welch Medical Library). The relationship between genes and diseases that have been mapped to the same chromosomal region are then identified through linkage analysis (coinheritance of physically adjacent genes).

The differences in the cDNA or genomic sequence between affected and unaffected individuals can also be determined. If a mutation is observed in some or all of the affected individuals but not in any normal individuals, then the mutation is likely to be the causative agent of the disease.

An additional embodiment of the invention relates to the administration of a pharmaceutical composition, in conjunction with a pharmaceutically acceptable carrier, excipient or diluent, for any of the therapeutic effects discussed above. Such pharmaceutical compositions may comprise, for example, a polypeptide set forth in Table 3 or 3A, antibodies to that polypeptide, mimetics, agonists, antagonists, inhibitors or other modulators of function of a Table 3 or 3A polypeptide or gene therefore. The compositions may be administered alone or in combination with at least one other agent, such as stabilizing compound, which may be administered in any sterile, biocompatible pharmaceutical carrier including, but not limited to, saline, buffered saline, dextrose and water. The compositions may be administered to a patient alone, or in combination with other agents, drugs or hormones.

In addition, any of the therapeutic proteins, antagonists, antibodies, agonists, antisense sequences or other modulators described above may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment, prevention or amelioration of pathological conditions associated with abnormalities in the APP pathway. Using this approach, one may be able to achieve therapeutic efficacy with lower dosages of each agent, thus reducing the potential for adverse side effects. Antagonists, agonists and other modulators of the human polypeptides set forth in Table 3 or 3A and genes encoding said polypeptides and variants thereof may be made using methods which are generally known in the art.

The pharmaceutical compositions encompassed by the invention may be administered by any number of routes including, but not limited to, oral, intravenous, intramuscular, intra-articular, intra-arterial, intramedullary, intrathecal, intraventricular, transdermal, subcutaneous, intraperitoneal, intranasal, enteral, topical, sublingual or rectal means.

In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well-known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato or other plants; cellulose, such as methyl cellulose, hydroxypropylmethyl-cellulose or sodium carboxymethylcellulose; gums including arabic and tragacanth; and proteins, such as gelatin and collagen. If desired, disintegrating or solubilizing agents may be added, such as the cross-linked polyvinyl pyrrolidone, agar, alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions and suitable organic solvents or solvent mixtures. Dyestuffs or pigments may be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches; lubricants, such as talc or magnesium stearate; and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution or physiologically-buffered saline. Aqueous injection suspensions may contain substances that increase the viscosity of the suspension, such as sodium carboxymethyl cellulose, sorbitol or dextran. Additionally, suspensions of the active compounds may be prepared as appropriate oily injection suspensions. Suitable lipophilic solvents or vehicles include fatty oils, such as sesame oil; or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly-concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping or lyophilizing processes.

The pharmaceutical composition may be provided as a salt and can be formed with many acids including, but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder that may contain any or all of the following: 1-50 mM histidine, 0.1-2% sucrose and 2-7% mannitol, at a pH range of 4.5-5.5, that is combined with buffer prior to use.

After pharmaceutical compositions have been prepared, they can be placed in an appropriate container and labeled for treatment of an indicated condition. Such labeling would include amount, frequency and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs or pigs. The animal model may also be used to determine the appropriate concentration range and route of administration. Such information can then be used to determine useful doses and routes for administration in humans.

A therapeutically-effective dose refers to that amount of active ingredient which ameliorates the symptoms or condition. Therapeutic efficacy and toxicity may be determined by standard pharmaceutical procedures in cell cultures or experimental animals, e.g., the dose therapeutically effective in 50% of the population (ED₅₀) and the dose lethal to 50% of the population (LD₅₀). The dose ratio between toxic and therapeutic effects is the therapeutic index, and it can be expressed as the ratio, LD₅₀/ED₅₀. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities and tolerance/response to therapy. Long-acting pharmaceutical compositions may be administered every 3-4 days, every week or once every two weeks depending on half-life and clearance rate of the particular formulation.

Normal dosage amounts may vary from 0.1-100,000 mg, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569 and 6,051,561.

The following Examples illustrate the present invention, without in any way limiting the scope thereof.

The following methods are employed to perform the examples provided below:

### DNA Constructs and Molecular Techniques

A Drosophila model of AD is described in detail in U.S. Patent No. US20020174446. Briefly, in an effort to mimic the disease-specific Aβ42 over-expression, transgenic flies whose genome comprises the GMR-Aβ42 amyloid transgene are created using the GMR fusion expression system in order to ectopically express the transgene in the developing Drosophila eye. In order to express the Aβ42-peptide in the Drosophila eye, the Aβ42-sequence is cloned into the pGMR vector which is directed to the eye tissue throughout the development of the eye, as well as during adulthood, making it a suitable system for expression of Aβ42.

In this model, ectopic over-expression of Aβ42 disrupts the regular trapezoidal arrangement of the photoreceptor cells of the ommatidia (identical single units, forming the Drosophila compound eye), and the severity of the disruption depends on transgene copy number such that introduction of more copies of the Aβ42 transgene in the Drosophila eye, reflected by increased levels of Aβ-protein, has an additive affect on the rough-eye phenotype. For example, it has been seen that with two copies of a Aβ-transgene (fly strain K18.3), in agreement with observations at the macroscopic level, eyes display variable disorganization. As the phenotype gets worse, the concentration of dense, staining masses around the ommatidia increases, as do the gaps in the tissue. The ommatidia look smaller and are missing photoreceptors. Two copies of a higher expressing transgene (fly strain KJ103) show a phenotype similar in severity. Finally, eyes from Drosophila expressing four copies of a strong expressing transgene (fly strain KJ54) show an almost complete loss of photoreceptors. Data also indicate a shift in the phenotypic severity of the Aβ expressing flies as they age.

### Genetic Crosses, Analysis and Visualization of Phenotypes

Flies are crossed according to conventional methods except that all crosses are kept at 29° C. for maximal expression of phenotypes. In the binary Gal4 expression system, this temperature maximizes activity of the Gal4 protein. In the case of pGMR-Aβ42, it is observed that the phenotype is stronger at 29° C., so these flies are kept at this temperature as well.

For 2^{nd} and 3^{rd} chromosome EP strains, crosses are set using male flies (2-3) from the EP strain and virgin females (3-4) from the Aβ over-expressing strain (KJ54). For X-chromosome EP strains, 3-4 virgin females are collected from individual EP strains and mated with 2-3 males from the KJ54 strain. The crosses are set up at 25° C. and females are allowed to lay eggs for 3-4 days followed by transfer to 29° C. The progeny are aged for 7-10 days and are scored for eye roughness using a dissecting microscope.

### S2 Cells Transfection and ELISA to Detect Aβ-Secretion

Drosophila tissue culture S2 cells are cultured overnight in 24-well plate at a density of 2×10⁵-4×10⁵ cells/mL/well at 25° C. Transfection mixture is prepared in a 17×100 mm polycarbonate tube by mixing 2 μg, 3 μg and 10-20 μg, respectively, of plasmids carrying MTGal4, UAS-DsRed (publicly available) and UAS Aβ40 DNA (constructed internally) constructs in a total volume of 360 μL. To this mixture 40 μL of 2.5 M CaCl₂ (10×) is added dropwise with swirling, followed by addition of 400 μL of 2×BBS in the same manner. The mixture is incubated for 30 min. at RT and 200 μL of the mixture is used per well of the 24-well plate. The cells are incubated with the transfection mixture or the BES buffer alone (control) for 24 hours and washed 2× with fresh medium. To induce MTGal4 gene, 1 μL of 0.7 M CuSO₄ is diluted with 1 mL of fresh medium and added to the wells containing transfected cells. Cells are harvested after 48 hours and then spun down to separate the supernatant. The pellet and the supernatant are frozen separately at −80° C. The human A40 colorimetric ELISA kit (Catalogue No. KHB3481) from Biosource International (Camarillo, Calif.) is used to assay the supernatants. The plate is washed 3× with the wash buffer provided in the kit. The standard peptide provided in the kit is used to make a serial dilution and 100 μL of standard peptide solution or S2 cell culture supernatant is added into appropriate wells of the pre-coated plate. The plate is incubated overnight at 4° C. on a flat surface in dark, without shaking. The plate is washed three times with 200 μL wash buffer/well followed by addition of 100 μL of diluted detection antibody (rabbit polyclonal anti-Aβ40, also from kit). The plate is incubated on a shaker (450 rpm) at RT for 2 hours, washed 3× with 200 μL wash buffer/well. Secondary antibody solution (HRP conjugated anti-rabbit antibody) is added to the wells at 100 μL/well followed by a 2 hours incubation at RT on a shaker. The wells are again washed three times with 200 μL wash buffer each and 100 μL stabilized chromogen solution is added to each well. The plate is incubated for 30 min. at RT, protected from light, and the reaction is stopped by adding 100 μL of stop solution to each well. Optical density is measured using a plate reader.

### In Vitro Translation Using RTS 500

Aβ42-peptide with the attached pre-proenkephalin signal sequence [see Cescato et al., J Neurochem, Vol. 74, No. 3, pp. 1131-1139 (2000)] is obtained by in vitro translation using Rapid Translation system 500 (RTS 500) from Roche, Indianapolis, Ind., Catalog No. 3018008). The pre-proenkephalin-Aβ42 construct is cloned into a template vector (pIVEX) designed for prokaryotic in vitro protein expression and containing a T7 promoter (contained in the RTS 500 kit). The resulting recombinant plasmid is used for continuous exchange cell-free protein synthesis (CECF) using RTS 500 system and following the manufacturer's instructions. In this system transcription and translation take place simultaneously in the 1 mL reaction compartment of the reaction device. Substrates and energy components essential for a sustained reaction are continuously supplied via a semi-permeable membrane. At the same time, potentially inhibitory reaction by-products are diluted via diffusion through the same membrane into the 10 mL feeding compartment. Protein is expressed for up to 24 hours yielding up to 500 μg of functionally active protein.

### Western Blot Analysis

Flies of desired genotype are frozen in an eppendorf tube using liquid nitrogen and quickly vortexed to severe heads from the bodies. The contents of the tube are dumped on a weighing boat kept on dry ice and the heads are separated from other body parts using a pre-cooled fine paintbrush. To extract proteins from 50-100 heads, 50 μL of 28× stock of Complete Protease Inhibitor Mini tablets (Roche, Catalog No. 1 836 153) (add first to frozen heads) and 200 μL 2× sample buffer B (0.318 M Bicine, 30% sucrose, 2% SDS, 0.718 M Bistris) are added to the fly heads. Samples are subsequently homogenized by hand using a plastic pestle, then heated at 95° C. for 5 min. in a dry bath incubator and spun in a microcentrifuge at 12 K rpm, 5 min., 25° C. The supernatant is transferred to a protease free tube (Biopur, SRL, Rosario, Argentina) using a pipette tip. Protein samples are quantitated using the Biorad (Hercules, Calif.) protein assay (according to manufacturer's instructions for standard assay in a microtiter plate). Five percent (5%) β-mercaptoethanol (2.5 μL for 50 μL) and 0.01% of Bromophenol blue (BB) (use 1 μL of 2% BB for 50 μL) are added to the samples. The samples are incubated at 100° C. in a dry bath incubator for 5 min. prior to loading. Fifty (50) μg of total protein extract is loaded for each sample, on a 15% tricine/tris SDS PAGE gel containing 8 M urea (gel composition provided below).

The gel used does not contain urea. Samples are run at 40 V in the stacking gel and at 120 V in the separating gel (about 1.5 hours). One (1) x tris-tricine/SDS (diluted from 10× stock from Biorad) buffer is used as a cathode buffer between the gels and 0.2 M tris-HCl, pH 8.8 (diluted from 1.5 M stock from Biorad) is used as an anode buffer on the bottom. The Aβ1-42 peptide control is human β-amyloid (142) (Biosource International, Camarillo, Calif., No. 03-111, Lot No. 0311219B). The peptide is dissolved at 1 μg/μL to make a stock. Prior to loading, an aliquot is diluted to 2 ng/μL concentration and mixed in 1:1 ratio with 2× sample buffer. Before loading, β-mercaptoethanol and BB are added at 5% and 0.01%, respectively. Molecular weight marker RPN 755 (Amersham, Piscataway, N.J.) is used as a size marker. It is prepared for loading in a similar fashion to peptide marker. After electrophoresis, samples are transferred to PVDF membranes (Biorad, No. 162-0174) for 1 hour at 100 V and the membranes are subsequently boiled in 1×PBS for 3 min. (with the membrane protein side down). The membranes are blocked with 5% non-fat milk prepared in 1×PBS containing 0.1% Tween 20 for 1.5 hours to overnight. Antibody hybridization is as follows: the primary mAb 6E10 (Senetek PLC, Napa, Calif.), which recognizes the first 19 amino acids of the Aβ-peptide, is used for probing (at a concentration of 1:1000) in 5% non-fat milk dissolved in 1×PBS containing 0.1% Tween-20, for 90 min. at RT. The membranes are washed 3× for 5 min., 15 min. and 15 min. each, in 1×PBS-0.1% Tween-20. The secondary Aβ is anti-mouse HRP (Amersham Pharmacia Biotech, Piscataway, N.J., No. NA 931) and is used at 1:2000 in 5% non-fat milk dissolved in 1×PBS containing 0.1% Tween-20, for 90 min. at RT. Samples are washed as the after primary antibody incubation. ECL (Western Blotting Detection Reagents, Amersham Pharmacia Biotech, No. RPN2209) is used for detection. After blotting, membranes are washed with water several times and stained with Ponceau reagent to confirm equal loading in all lanes.

15% tricine/tris SDS PAGE gel containing 8 M urea (for 2 gels)

| | Separating | | Stacking | |
|---|---|---|---|---|
| | Gel | | Gel* | |
| Urea | 4.8 | g (dissolve) | — | |
| (30/0.8) % Acr/Bis | 5.0 | mL | 512 | μL |
| 3× gel buffer | 3.334 | mL | 1 | mL |
| (3 M Tris/Cl, | | | | |
| ph 8.45; 0.3% SDS) | | | | |
| 10% SDS | 100 | μL | 100 | μL |
| Water | 0 | | 2.48 | mL |
| | | ↓ | | |
| | Filter using 0.45μ disposable syringe filter | | | |
| 10% APS | 50 | μL | 32 | μL |
| TEMED | 5 | μL | 3.2 | μL |

*There is no need to filter the stacking gel solution.

### Immunostaining

Eye-antennal imaginal discs are dissected from 3^{rd} instar larvae of desired genotype in 1×PBS solution using a dissecting microscope. Tissue is fixed for 30 min. at RT in 3% paraformaldehyde prepared in 1×PBS. The fixative is removed by washing 3× with 1×PBS containing 0.1% triton X-100 (1×PBST). Tissue is blocked for 1 hour at RT using 2% BSA made in 1×PBST. After washing 3× with 1×PBST, primary antibody (mAb 6E10 from mouse, Senetek PLC) is added at a dilution of 1:3000 followed by overnight incubation at 4° C. After 3 washes with 1×PBST, fluorescent secondary antibody (anti-mouse Alexa-488 at 1:300 dilution, Molecular Probes, Eugene, Oreg.) and fluorescent phalloidin (Alexa-568 Phalloidin at 1:30 dilution, Molecular Probes) are added and incubated at RT for 1 hour. The tissue is washed 3× with 1×PBST and mounted using slow-fade light anti-fade medium (Molecular Probes). The slides are analyzed using a Biorad confocal microscope and images are collected using Lasersharp 4.1 software (Biorad).

### EXAMPLE 1

### Genetic Scheme for the Primary Screen Using EP Insertion Lines

A Drosophila model for AD is created by over-expression of the Aβ42-peptide using the eye-specific GMR promoter (see methods section above). The construct contains the Aβ42-coding region fused to the pre-proenkephalin signal peptide that has been shown to mediate secretion of Aβ42 from transfected mammalian cells. See Cescato (2000), supra. Data previously indicate that Aβ42 effects in Drosophila are dose-dependent. In order to be able to identify mutations that both enhance and suppress the Aβ42-phenotype, we chose to use for the genetic screen a Aβ42-strain with relatively high levels of transgenic expression. This Aβ42-strain, designated KJ54 carries Aβ42-transgenes on both 2^{nd} and 3^{rd} chromosomes and shows a distinct rough-eye phenotype at 25° C. This phenotype becomes worse when flies are reared at 29° C. The temperature dependence of the rough-eye phenotype makes the KJ54-strain suitable for our intended purposes. In order to use this strain for an EP-based genetic screen, the eyGal4 was recombined on the 2^{nd} chromosome of the fly-strain KJ54. Under the control of the eyGAL4, GFP expression can be detected in the whole eye imaginal disc of 3^{rd} instar larvae. Therefore, it is expected that eyGal4 would drive the mis-expression of the genes linked to the EP in a pattern similar to that of the GMR-driven Aβ42-expression.

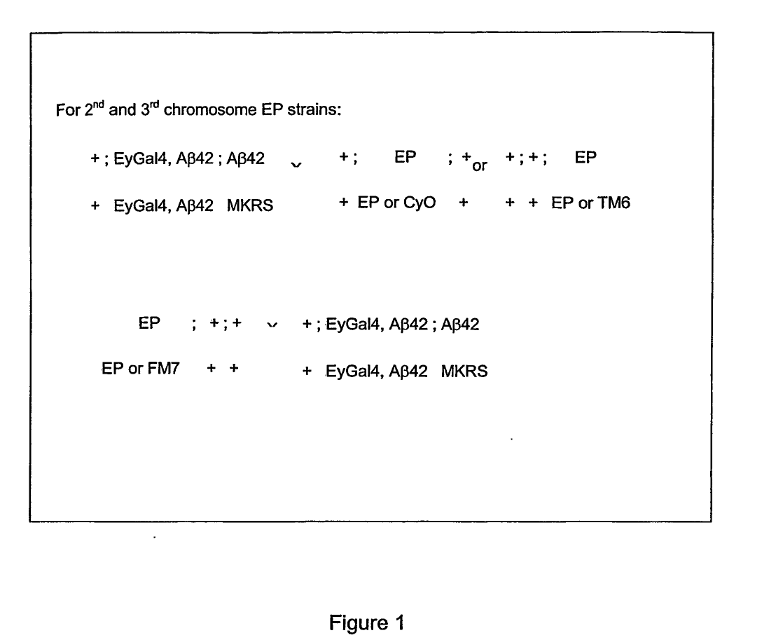

The typical parental cross schemes used for the genetic screen are given in FIG. 1. The experimental progeny from these crosses carry copies of the Aβ-transgene on chromosome 2 and 3 and a copy of the EP element on one of the sister chromosomes. These are compared to the control progeny that have copies of Aβ-transgene on chromosome 2 and 3 but no EP on the sister chromosomes. The degree of roughness is compared between the experimental and control class of progeny. Any suppression or enhancement of eye roughness caused by EP element driven mis-expression of genes is classified into mild, moderate and strong categories.

### EXAMPLE 2

### Neprilysin Mis-Expression Strongly Suppresses the Aβ42-Induced Rough-Eye Phenotype

In order to use our model system for an EP based genetic screen we recombined the eyGal4 gene on the 2^{nd} chromosome. But before using this model in a large scale genetic screen, we decided to test some candidate genes to determine the efficacy of this system in revealing biologically relevant interactions.

Neprilysin, a member of zinc metallopeptidase family, is strongly implicated in Aβ-catabolism in mammalian brain. See Selkoe, Neuron, Vol. 32, No. 3, pp. 177-180 (2001); and Carson and Turner, J Neurochem, Vol. 81, No. 1, pp. 1-8 (2002). Neprilysin-like activities are well-conserved from prokaryotes to man. At least 24 neprilysin-like genes are reported to be present in Drosophila melanogaster. See Turner et al., Bioessays, Vol. 23, No. 3, pp. 261-269 (2001). In order to determine the effectiveness of our genetic screen, a strain carrying an insertion near a fly neprilysin gene, for modification of the rough-eye phenotype may be tested. EP(3)3549 is inserted upstream of the fly neprilysin 2 (Nep2) gene. Upon progeny analysis, a clear and strong suppression of the rough-eye phenotype is observed in flies carrying both the Aβ42-transgenes and EP(3)3549, as compared to flies carrying only the Aβ42-transgenes (data not shown). This data is significant as it indicates conservation of an important aspect of Aβ-catabolism between humans and flies. This result also gave us confidence in the robustness of the genetic screen.

To test if the co-expression of neprilysin in our transgenic model affects turnover of the Aβ42-peptide, immunostains of eye imaginal discs from 3^{rd} instar larvae with an Aβ-specific antibody is performed. Data indicate that co-expression of the Nep2 gene eliminated most of the Aβ42-peptide from the eye imaginal discs. Also performed are western blot analysis using transgenic fly head protein extracts. Results indicate that the levels of Aβ-peptide in flies expressing both Aβ42 and neprilysin are dramatically reduced, as compared to flies expressing only Aβ42 (data not shown).

Thus, suppression of the rough-eye phenotype by neprilysin and cell biological and biochemical evidence that the enzyme degrades the Aβ42-peptide in the fly eye, strongly suggest that genes involved in the development and/or progression of conditions involving the aberrant metabolism, trafficking or turnover of Aβ, including AD, may be elucidated by using our fly AD model in a genetic screen.

### EXAMPLE 3

### Aβ42-Peptide is Secreted by the Photoreceptor Cells in the Eyes of Transgenic Flies

Neprilysin belongs to a family of ectopeptidases that are membrane bound and contain an extracellular enzymatic domain. See Turner et al., (2001), supra. The neprilysin family of enzymes are known to degrade neuropeptides in the extracellular space. Based on this information about neprilysin function, we hypothesized that the Aβ42-peptide is secreted by the fly photoreceptor cells. Such secretion of Aβ42 could lead to degradation of the peptide by neprilysin, in the extracellular space. To test this hypothesis, processing of pre-proenkephalin signal peptide in protein extracts from heads of transgenic flies is first examined. In order to distinguish between Aβ-peptides with and without the signal sequence, in vitro translated protein is prepared from the same pre-proenkephalin Aβ42-construct that is used to generate transgenic flies. The in vitro translated product is compared to commercially-available (Biorad) synthetic Aβ42-peptide (without the signal sequence). Data indicate that a clear migration shift is caused by the presence of the signal peptide. When Aβ42-peptide from transgenic fly extract is analyzed, we observe that it co-migrates with the signal sequence-free Aβ-peptide, suggesting processing of transgenic Aβ42 in the photoreceptor cell secretory pathway.

The secretion of Aβ42 in cultured S2 cells (ATCC, Manassas, Va.) after transiently transfecting with our pre-proenkephalin Aβ40-transgene construct is tested. ELISA is performed on the supernatant of transiently transfected S2 cells to detect presence of secreted Aβ40-peptide. Results show detection of Aβ40 in the supernatant of cells expressing Aβ40.

Taken together, these results indicate that Aβ-peptide can be secreted in flies, suggesting that the toxic effects on the eye tissue may be mediated by extracellular Aβ42.

### EXAMPLE 4

### Genetic Screen to Find Genes that Modify the Aβ42 Over-Expression Dependent Rough-Eye Phenotype

A genetic screen using our model system to reveal novel genetic interactions that would enhance or suppress the Aβ42-induced rough-eye phenotype is conducted. The screen utilize a publicly-available collection of EP insertion stocks. The recombinant KJ54 strain with the eyGal 4 on the 2^{nd} chromosome is crossed individually to 1,967 EP strains and the progeny is scored for the changes in eye roughness with suppressors and enhancers of the rough-eye phenotype being divided into three subcategories: strong, moderate and mild, depending on the strength of modification. After a primary screen, 238 enhancers (30 strong, 48 moderate and 160 mild) and 97 suppressors (7 strong, 17 moderate and 73 mild) are obtained.

In order to confirm that the eyGal4-driven expression of EP-strains by itself does not give a rough-eye phenotype (or any other kind of eye defect), 102 strong and moderate modifiers (enhancers and suppressors) are crossed to the flies carrying only the eyGal4 insertion on the 2^{nd} chromosome. In parallel to this genetic background check, re-screen crosses of the 102 EP-strains are also performed with the KJ54-strain to confirm the results from the primary screen. In the re-screen, we also include an EP sitting in the 5′ region of IDE even though it was scored as a mild suppressor in the primary screen. Another mild enhancer EP-strain, EP(X)356, was also re-tested. This EP-insertion is expected to affect the gene silver, which is a fly homologue of the human carboxypeptidase Z gene, which we have shown increases the levels of secreted Aβ42 in the supernatant of cultured mammalian cells transiently transfected with the APP gene. In addition to the re-screen, we also cross these 104 EP strains to flies carrying the eyGal4 alone to determine if the mis-expression of an EP-strain by itself gives an eye phenotype, such as change in size, shape or roughness. These two sets of parallel experiments confirm that 23 EP-strains have reproducible and specific effects on the Aβ42-induced rough-eye phenotype (see Table 1). The 23 genes shown in Table 1 are exemplary sequences of genes affected by the mutations carried in the EP-strains. Additional sequences which include variants of these genes and the proteins/polypeptides they encode, not shown here, are also included as additional targets covered by this invention.

TABLE 1
Annotation for 23 Modifier EP Strains

| | | | | Background | | |
|---|---|---|---|---|---|---|
| | | | Functions/Putative | Test/Cross | Re- | Modifier |
| EP No. | E/S | Gene/Annotation | functional domains | to eyGal | Screen | of Aβ42 |
| EP(2)0684 | Strong E | Escargot (CG3758) | A specific RNA | No effect | Moderate | Yes |
| | | GOF | polymerase II | | E | |
| | | | transcription factor | | | |
| | | | It acts as | | | |
| | | | transcriptional | | | |
| | | | repressor | | | |
| | | | Expression in the | | | |
| | | | neurogenic region and | | | |
| | | | antagonism of Scute | | | |
| | | | and Daughterless | | | |
| | | | suggest that escargot | | | |
| | | | opposes a proneural | | | |
| | | | fate | | | |
| | | | esg is a key regulator | | | |
| | | | of cell adhesion and | | | |
| | | | motility in tracheal | | | |
| | | | morphogenesis | | | |
| EP(2)0965 | Strong E | ElbowB (CG4220) | RNA polymerase II | Small eye | Moderate | Yes |
| | | | transcription factor | | E | |
| | | | EIB is expressed in | | | |
| | | | specific subset of | | | |
| | | | tracheal cells and | | | |
| | | | specifies distinct | | | |
| | | | tracheal branching | | | |
| | | | fates | | | |
| | | | EIB associates with | | | |
| | | | Noc to form | | | |
| | | | heterodimer | | | |
| | | | EIB may repress | | | |
| | | | transcription of target | | | |
| | | | genes involved in | | | |
| | | | tracheal branching | | | |
| EP(2)0330 | Moderate | P{EP}EP330 | Unknown | No effect | Mild/ | Yes |
| | E | | | | Moderate | |
| | | | | | E | |
| EP(2)0386 | Moderate | Mis-expression | Transcription factor | Small eye | Moderate | Yes |
| | E | suppressor of | Zinc finger | | E | |
| | | ras 4 (CG4903) | C₂H₂ type | | | |
| | | GOF (571 bp | Elongation factor Ts | | | |
| | | up-stream) | (EF-Ts) | | | |
| | | | Dimerization domain | | | |
| EP(2)2510 | Moderate | CG7231 | Unknown | No effect | Mild E | Yes |
| | E | LOF (205 bp from | | | | |
| | | ATG within 5′ of the | | | | |
| | | gene) | | | | |
| EP(3)1051 | Strong E | CG5490 | TI receptor signaling | Small eye | Mild E | Yes |
| | | Toll receptor | pathway/defense | | | |
| | | (FlyDB) | (immune) response | | | |
| | | No information in | | | | |
| | | Flybase | | | | |
| | | GOF (64 bp | | | | |
| | | up-stream to TI) | | | | |
| EP(3)3015 | Strong E | 2 insertions | Protein serine/ | Small eye | Moderate | yes |
| | | depicted in | threonine kinase (for | | E | |
| | | Flybase | EP(3)3015b) | | | |
| | | 3015a and b! | | | | |
| | | 3015a has no info | | | | |
| | | 3015b inserted | | | | |
| | | within SNF4/ | | | | |
| | | AMP-activated | | | | |
| | | protein kinase | | | | |
| | | γ subunit or | | | | |
| | | SNF4Aγ (CG17299) | | | | |
| | | gene (LOF or | | | | |
| | | GOF?) | | | | |
| EP(3)3549 | Strong S | Neprilysin 2 | It encodes a | No effect | Strong S | yes |
| | | GOF (93 bp | metallopeptidase | | | |
| | | up-stream on the | | | | |
| | | same strand) | | | | |
| EP(3)0595 | Moderate | CG6745 (LOF, | CG6745 is unknown | Small eye | Mild S | Yes |
| | S | sitting within the | CG6765 is | | | |
| | | gene at 3′ end) or | transcription factor | | | |
| | | CG 6765 (GOF, | | | | |
| | | sitting 2.35 kb | | | | |
| | | up-stream to this | | | | |
| | | gene) | | | | |
| EP(3)3041 | Moderate | FLYdb plot: | Unknown | Small eye | Likely | Re-Test |
| | S | nearest down- | | | mild S | |
| | | stream gene on | | | (semi | |
| | | the same strand is | | | lethal; few | |
| | | CG14959 at 16.3 | | | exptal | |
| | | kb (GOF?) | | | males) | |
| EP(3)3108 | Moderate | CG7437 | It encodes a poly(rC) | No effect | Mild S | Yes |
| | S | Mushroom-body | binding | | | |
| | | expressed | | | | |
| | | LOF? (843 bp within | | | | |
| | | 5′ of the gene on | | | | |
| | | opposite strand) | | | | |
| EP(3)3348 | Mild/ | Gene CG10967 | Protein serine/ | No effect | Mild S | Yes |
| | Moderate | (FlyBase) | threonine kinase | | | |
| | S | CG11006 (FlyDB) | (CG10967) | | | |
| | | LOF for CG10967 | Ribosomal protein | | | |
| | | (100 bp within 5′ on | (CG11006) | | | |
| | | opposite strand) | | | | |
| | | GOF for CG11006 | | | | |
| | | (961 bp up-stream of | | | | |
| | | it) | | | | |
| EP(3)3405 | Mild/ | CG6175 (FlyBase) | Unknown | No effect | Mild S | Yes |
| | Moderate | GOF (2 kb | | | | |
| | S | up-stream of it) | | | | |
| EP(3)3470 | Mild/ | No information on | Unknown | No effect | Mild S | Yes |
| | Moderate | FlyBase or FlyDB | | | | |
| | S | | | | | |
| EP(3)3603 | Moderate | LOF for CG6767 | CG6767 encodes a | No effect | Moderate | Yes |
| | S | (1.1 kb within 5′ of | ribose-phosphate | | S | |
| | | the gene on | pyrophosphokinase | | | |
| | | opposite strand) or | CG8284 encodes | | | |
| | | GOF for CG8284 | ubiquitin conjugating | | | |
| | | (2.7 kb up-stream of | enzyme | | | |
| | | it) | | | | |
| EP(3)3099 | Mild S | GOF for CG5517 | CG5517 encodes | No effect | Mild S | Yes |
| | | (496 bp up-stream of | insulin-degrading | | | |
| | | it) | enzyme | | | |
| | | LOF for CG5701 | CG5701 encodes | | | |
| | | (77 bp within 5′ of | RHO small GTPase | | | |
| | | this gene on | | | | |
| | | opposite strand) | | | | |
| EP(X)1504 | Moderate | Unknown | Unknown | No effect | Mild E | Yes |
| | E | | | | | |
| EP(X)1318 | Moderate | Unknown | Unknown | Slightly | Mild E | Yes |
| | E | | | smaller | | |
| | | | | eyes, no | | |
| | | | | roughness | | |
| EP(X)0514 | Strong S | Garnet (CG11197) | Encodes a product | No effect | Moderate | Yes |
| | | LOF (inserted | involved in ocellus | | S | |
| | | 5.9 kb within the | pigment biosynthesis | | (glassy eyes | |
| | | gene on opposite | | | w/o necrotic | |
| | | strand) | | | spots in | |
| | | | | | males; | |
| | | | | | females | |
| | | | | | have some | |
| | | | | | necrotic | |
| | | | | | spots) | |
| EP(X)0355 | Moderate | Dorsal switch | It encodes a | No effect | Mild S | Yes |
| | S | protein 1 of DSP1 | transcription co- | | | |
| | | (CG12223) | repressor/single- | | | |
| | | LOF? (13 bp within | strand DNA binding | | | |
| | | 5′ of the gene on the | | | | |
| | | same strand) | | | | |
| EP(X)1596 | Moderate | EG:25E8.2 | It encodes an ubiquitin | No effect | Moderate | Yes |
| | S | (CG2924) | conjugating enzyme | | S | |
| | | LOF (inserted | involved in ubiquitin | | | |
| | | 1.6 kb within the 5′ | cycle | | | |
| | | of gene on opposite | | | | |
| | | strand) | | | | |
| EP(X)0308 | Mild/ | LOF for CG1886 | CG1886 encodes a | No effect | Mild S | Yes |
| | Moderate | (4 bp up-stream of | copper exporting | | (eyes little | |
| | S | this gene on the | ATPase | | rounder) | |
| | | opposite strand) | CG10347 has hsp-20 | | | |
| | | GOF for CG10347 | like chaperone domain | | | |
| | | (304 up-stream of it | | | | |
| | | on the same strand) | | | | |
| EP(X)0356 | Mild E | Silver (CG18503) | Fly carboxypeptidase | No effect | Mild E | Yes |
| | | LOF (inserted within | Z homolog | | | |
| | | the gene on the | | | | |
| | | same strand) | | | | |

E/S = Enhancer/Suppressor

### EXAMPLE 5

### Human Homologues of the 23 EP Modifiers

In parallel to ongoing validation assays for the 23 Aβ42-modifiers in the Drosophila system, bioinformatics analysis is used to determine the human homologues/orthologues of the fly genes affected by these modifier EP-insertions. The insertion site for three out of the 23 EP-modifiers [EP(2)0330, EP(X)1318 and EP(X)1504] is not available and thus the affected gene in these strains is unknown. Pending further analysis of the effect of these modifier EP-strains by western analysis or other secondary assays, the insertion site of the EP-element will be obtained by inverse PCR or plasmid rescue and sequencing.

The nature of the EP-element is such that it can over-express a gene only if it is inserted upstream of the gene and in the same orientation of transcription of the gene itself. EP-insertions that do not fulfill the above criteria are expected to disrupt the transcription of the affected gene. To analyze the nature of the mutations caused by each EP-insertion, information is gathered from Flybase and FlyDB3, a proprietary database, in order to find the four neighboring genes immediately next to the insertion. Based on the relative distance from the insertion and the orientation, the genes that can possibly be affected by the EP-insertion are picked up and used for Blast analysis. Twenty-four (24) genes are found to be in the vicinity of the 20 EPs, such that could potentially be affected by the EP-insertions. These 24 genes are subjected to Blast analysis according to conventional methods. Parameters for the mapping of the Drosophila melanogaster protein sequences to Refseq, Celera and Compugen protein sequences are as follows: the E-value should be <10e-10 and >25% of the query sequence's length is part of the alignment. Refseq release April 2002 and Celera proteins R26j are used as the databases for the Blast analysis. Homologous sequences are found for 18/24 genes in the Refseq database and for 20/24 genes in the Celera proprietary database.

Annotations are found for 16/20 human homologues in the Celera database (see Table 2). Twelve out of twenty (12/20) of the human homologues when blasted back to Drosophila protein database, pull out our primary hit sequences as suitable matches (highlighted in bold), indicating that they may be orthologues. In addition, chromosomal location for human homologues of these fly genes will be used to determine if there is any linkage of AD to these map locations.

Full-length cDNA clones are available for 13/20 of the available human homologues (see Table 3 or 3A). The clones highlighted in bold represent the re-arrayed clones. Apart from the re-arrayed clones, the order of clones represents the quality of the hit. For 3 of the genes not covered in our proprietary libraries, full-length clones are available from MGC (public database), extending the availability of full-length cDNA clones to 16/20 genes.

TABLE 2
Annotations for the Human Homologues of the 23 Modifier EP-Strains

| | | Homosapiens | | | | |
|---|---|---|---|---|---|---|
| Drosophila Melanogaster | | | | Panther | Chromo- | |
| | Fly | Refseq | Celera | annotation | somal | Compugen |
| Modifier | gene(s) | protein | protein | (Celera) | location | transcript |
| EP(2)0684 | CG3758 | | hCP46345.1 | Escargot/snail | 8q11.1 | R24694_TR_1 |
| | | | | protein | | |
| EP(2)0965 | CG4220 | NP_079345.1 | hCP1763153 | | 10q22 | R72623_TR_3 |
| EP(2)0330 | Unknown | | | | | |
| EP(2)0386 | CG4903 | <————————— No homologue found —————————> | | | | |
| EP(2)2510 | CG7231 | | hCP47876.1 | | 5q13 | R92398_TR_1 |
| EP(3)1051 | CG5490 | NP_570843.1 | hCP1617715.1 | Leucine rich | 3q29 | HUMCARN_TR_2 |
| | | | | repeat protein | | |
| | | | | (gb definition: | | |
| | | | | carboxy- | | |
| | | | | peptidase N | | |
| | | | | 83 kda chain) | | |
| EP(3)3015 | CG17299 | NP_057287.1 | hCP1769289 | 5′-AMP- | 7q36 | M78939_TR_17 |
| | | | | activated protein | | |
| | | | | kinase, γ-1 | | |
| | | | | subunit | | |
| EP(3)3549 | CG9761 | NP_258428.1 | hCP41722.2 | Neprilysin | 1p36.3 | AW845925_TR_1 |
| EP(3)0595 | CG6745 | NP_112582.1 | hCP1765749 | | 7q22 | H63270_TR_3 |
| | CG6765 | <————————— No homologue found —————————> | | | | |
| EP(3)3041 | CG14959 | <————————— No homologue found —————————> | | | | |
| EP(3)3108 | CG7437 | NP_065389.1 | hCP801177.2 | Heterogeneous | 21q22 | AA704558_TR_8 |
| | | | | nuclear | | |
| | | | | ribonucleo- | | |
| | | | | protein | | |
| EP(3)3348 | CG10967 | NP_003556.1 | hCP44933.2 | Serine/ | 17p11.2 | T33475_TR_18 |
| | | | | threonine- | | |
| | | | | protein kinase | | |
| | | | | Ulk | | |
| | CG11006 | NP_078821.2 | hCP1763735 | Mucin-related | 2q14.2 | T08369_TR_4 |
| EP(3)3405 | CG6175 | <————————— No homologue found —————————> | | | | |
| EP(3)3470 | CG17100 | NP_036391.1 | hCP38867.1 | Basic helix- | 6q22-23 | N46845_TR_5 |
| | | | | loop-helix | | |
| | | | | transcription | | |
| | | | | factor, hairy/ | | |
| | | | | enhancer-of- | | |
| | | | | split-related | | |
| EP(3)3603 | CG6767 | NP_002755.1 | hCP1777740 | Ribose- | Xq22 | R13733_TR_9 |
| | | | | phosphate | | |
| | | | | pyrophos- | | |
| | | | | phokinase | | |
| | CG8284 | NP_005330.1 | hCP48441.2 | Ubiquitin- | 4p14 | Z40371_TR_17 |
| | | | | conjugating | | |
| | | | | enzyme | | |
| EP(3)3099 | CG5517 | NP_004960.1 | hCP1752293 | IDE | 10q24 | T11188_TR_9 |
| EP(X)1504 | Unknown | | | | | |
| EP(X)1318 | Unknown | | | | | |
| EP(X)0514 | CG11197 | NP_003929.2 | hCP40180.2 | AP-3 δ-adaptin- | 19p13.3 | M78381_TR_30 |
| | | | | related | | |
| EP(X)0355 | CG12223 | NP_002119.1 | hCP1767130 | High mobility | 13q12 | HSHMG1_TR_10 |
| | | | | group protein | | |
| | | | | HMG1-related | | |
| EP(X)1596 | CG2924 | NP_060052.1 | hCP45236.1 | NICE-5 protein | 1q12-21 | Z41193_TR_24 |
| EP(X)0308 | CG1886 | NP_000043.1 | hCP37790.2 | Copper- | Xq13 | HSMNKMBP_TR_{—} |
| | | | | transporting | | 5 |
| | | | | ATpase | | |
| | CG10347 | | hCP35819.2 | | 8q23 | T99380_TR_5 |
| EP(X)0356 | CG18503 | NP_001295.1 | hCP43520.2 | Carboxy- | 17q11.1 | T77473_TR_8 |
| | | | | peptidase D | | |

Mapping to human sequences: Parameters for the mapping of the D. melanogaster protein sequences to Refseq, Celera and Compugen protein sequences: E-value <10e−10; >25% of the query sequence's length is part of the alignment; Refseq release April 2002, Celera proteins R26j.
The IDs of reciprocal best matches are highlighted in bold
*Top hit in refseq is toll like receptor 3, (NM_003265/NP_003256.1), but leucine-rich repeat protein has biologically more meaningful alignment.
**<40 of the query sequence part of the alignment but very significant hit; huntingtin interacting protein is second best hit.

TABLE 3
Human cDNA Clones Homologous to the Fly Modifier Genes
(proprietary in house (FGA) and MGC collections)

| Drosophila | | Homosapiens | | |
|---|---|---|---|---|
| Melanogaster | | Compugen | FGA full-length | |
| Modifier | Fly gene(s) | transcript | clones | MGC full-length clones |
| EP(2)0684 | CG3758 | R24694_T1 | fga0000017816 | MGC:10182; IMAGE:3908245 |
| | | | fga0000231309 | MGC:17388; IMAGE:3911047 |
| | | | fga0000293514 | |
| | | | fga0000205695 | |
| | | | fga0000004196 | |
| | | | fga0000105112 | |
| | | | fga0000016211 | |
| | | | fga0000081041 | |
| | | | fga0000097611 | |
| | | | fga0000107772 | |
| | | | fga0000019254 | |
| | | | fga0000080311 | |
| | | | fga0000021444 | |
| | | | fga0000271368 | |
| | | | fga0000009265 | |
| EP(2)0965 | CG4220 | R72623_T3 | fga0000094341 | MGC:10743; IMAGE:4053098 |
| | | | fga0000086983 | MGC:4159; IMAGE:3604473 |
| | | | fga0000202920 | MGC:16268; IMAGE:3830632 |
| | | | fga0000195529 | MGC:2555; IMAGE:2967616 |
| | | | fga0000299196 | |
| | | | fga0000258179 | |
| | | | fga0000197152 | |
| | | | fga0000189525 | |
| | | | fga0000200817 | |
| | | | fga0000046685 | |
| | | | fga0000211211 | |
| | | | fga0000187723 | |
| | | | fga0000091007 | |
| EP(2)0330 | Unknown | | | |
| EP(2)0386 | CG4903 | <———————— No homologue found ————————> | | |
| EP(2)2510 | CG7231 | R92398_T1 | | MGC:24088; IMAGE:4608844 |
| | | | | MGC:27169; IMAGE:4614435 |
| EP(3)1051 | CG5490 | HUMCARN_T2 | | |
| EP(3)3015 | CG17299 | M78939_TR_17 | fga0000211832 | MGC:21127 IMAGE:4413055 |
| | | | fga0000294355 | |
| EP(3)3549 | CG9761 | AW845925_T1 | | MGC:26818; IMAGE:4812314 |
| EP(3)0595 | CG6745 | H63270_T3 | fga0000063021 | MGC:17720; IMAGE:3870711 |
| | | | | MGC:12222; IMAGE:3686082 |
| | CG6765 | <———————— No homologue found ————————> | | |
| EP(3)3041 | CG14959 | <———————— No homologue found ————————> | | |
| EP(3)3108 | CG7437 | AA704558_T8 | fga0000198091 | MGC:19901; IMAGE:4637826 |
| EP(3)3348 | CG10967 | T33475_T18 | fga0000093146 | |
| | CG11006 | T08369_T4 | fga0000090986 | MGC:16871; IMAGE:3906788 |
| EP(3)3405 | CG6175 | <———————— No homologue found ————————> | | |
| EP(3)3470 | CG17100 | N46845_T5 | | MGC:10720; IMAGE:3945225 |
| EP(3)3603 | CG6767 | R13733_T9 | fga0000102636 | MGC:2256; IMAGE:3542584 |
| | | | fga0000185231 | |
| | | | fga0000291594 | |
| | | | fga0000303990 | |
| | | | fga0000259623 | |
| | | | fga0000279818 | |
| | | | fga0000262198 | |
| | | | fga0000210198 | |
| | | | fga0000305275 | |
| | | | fga0000008039 | |
| | | | fga0000103384 | |
| | | | fga0000194842 | |
| | | | fga0000084636 | |
| | | | fga0000265181 | |
| | | | fga0000286723 | |
| | CG8284 | Z40371_T17 | fga0000080248 | MGC:39129; IMAGE:4893778 |
| | | | fga0000035551 | MGC:12679; IMAGE:3946309 |
| EP(3)3099 | CG5517 | T11188_T9 | | |
| EP(X)1504 | Unknown | | | |
| EP(X)1318 | Unknown | | | |
| EP(X)0514 | CG11197 | M78381_T30 | fga0000067775 | MGC:19566; IMAGE:4106961 |
| EP(X)0355 | CG12223 | HSHMG1_T10 | fga0000221354 | MGC:32637; IMAGE:4041682 |
| | | | fga0000232492 | MGC:5223; IMAGE:2901382 |
| | | | fga0000251775 | |
| | | | fga0000252357 | |
| | | | fga0000273016 | |
| | | | fga0000010937 | |
| | | | fga0000287919 | |
| | | | fga0000265953 | |
| | | | fga0000265351 | |
| | | | fga0000257400 | |
| | | | fga0000254596 | |
| | | | fga0000295152 | |
| | | | fga0000228662 | |
| | | | fga0000192077 | |
| | | | fga0000272662 | |
| | | | fga0000257952 | |
| | | | fga0000036586 | |
| | | | fga0000015502 | |
| | | | fga0000253362 | |
| | | | fga0000069106 | |
| EP(X)1596 | CG2924 | Z41193_T24 | fga0000194115 | MGC:14087; IMAGE:3927447 |
| | | | fga0000084481 | MGC:21081; IMAGE:4151953 |
| | | | fga0000069604 | MGC:12351; IMAGE:4052438 |
| | | | fga0000274961 | MGC:22826; IMAGE:3828885 |
| | | | fga0000249478 | MGC:24777; IMAGE:4284921 |
| | | | fga0000248984 | |
| | | | fga0000249359 | |
| | | | fga0000003389 | |
| | | | fga0000021026 | |
| | | | fga0000271287 | |
| | | | fga0000268412 | |
| | | | fga0000261012 | |
| | | | fga0000286752 | |
| | | | fga0000275982 | |
| | | | fga0000270423 | |
| | | | fga0000258419 | |
| | | | fga0000256011 | |
| | | | fga0000254306 | |
| | | | fga0000248357 | |
| | | | fga0000220199 | |
| EP(X)0308 | CG1886 | HSMNKMBP_T5 | | |
| | CG10347 | T99380_T5 | fga0000206930 | |
| | | | fga0000258557 | |
| | | | fga0000203101 | |
| | | | fga0000247362 | |
| EP(X)0356 | CG18503 | T77473_T8 | | |
| | | | fga0000086983 | |
| | | | fga0000094341 | |

Identification of full-length clones: ≧150 nt overlap; >=98% sequence identity, using the best matching Compugen transcript as a query sequence.
Apart from the re-arrayed FGA fl-clones which are highlighted in blue, the order of the clones represents the quality of the hits; clones in adjacent row reflects their ranking and does not imply that they correspond to each other.
Intersection with set of 478 full-length clones tested by Alex.

TABLE 3A
Human cDNA Clones Homologous to the Fly-Modifier

| Modifier | Fly Gene | Refseq ID | Internal SEQ ID NO: |
|---|---|---|---|
| EP(2)0684 | CG3758 | NM_003068 | 1 |
| | | | 2 |
| EP(2)0965 | CG4220 | NM_032772 | 3 |
| | | | 4 |
| EP(2)2510 | CG7231 | NM_176782 | 5 |
| | | | 6 |
| EP(3)1051 | CG5490 | NM_024950 | 7 |
| | | | 8 |
| EP(3)3015 | CG17299 | NM_016203 | 9 |
| | | | 10 |
| EP(3)3549 | CG9761 | NM_033467 | 11 |
| | | | 12 |
| EP(3)0595 | CG6745 | NM_019042 | 13 |
| | | | 14 |
| EP(3)3108 | CG7437 | NM_020528 | 15 |
| | | | 16 |
| EP(3)3348 | CG10967 | NM_003565 | 17 |
| | | | 18 |
| EP(3)3348 | CG11006 | NM_024545 | 19 |
| | | | 20 |
| EP(3)3470 | CG17100 | NM_012259 | 21 |
| | | | 22 |
| EP(3)3603 | CG6767 | NM_002764 | 23 |
| | | | 24 |
| EP(3)3603 | CG8284 | NM_005339 | 25 |
| | | | 26 |
| EP(3)3099 | CG5517 | NM_004969 | 27 |
| | | | 28 |
| EP(X)0514 | CG11197 | NM_003938 | 29 |
| | | | 30 |
| EP(X)0355 | CG12223 | NM_002128 | 31 |
| | | | 32 |
| EP(X)1596 | CG2924 | NM_017582 | 33 |
| | | | 34 |
| EP(X)0308 | CG1886 | NM_000052 | 35 |
| | | | 36 |
| EP(X)0308 | CG10347 | NM_032869 | 37 |
| | | | 38 |
| EP(X)0356 | CG18503 | NM_001304 | 39 |
| | | | 40 |

### EXAMPLE 6

### Mammalian Cell-Based Assay of Human cDNA and Effects on Aβ-Secretion

All the human cDNAs will be tested in our mammalian cell-based assay for effects on the levels of secreted Aβ. This assay may be performed according to the protocol described in Haugabook et al. (2001) J. Neurosci. Methods 108: 171-179

What is claimed is:

1. A method to treat, prevent or ameliorate neurodegenerative conditions comprising administering to a subject in need thereof an effective amount of a modulator of a protein selected from the group consisting of those disclosed in Table 3 or 3A.

2. The method of claim 1, wherein said condition is Alzheimer's Disease (AD).

3. The method of claim 1, wherein said modulator inhibits the biochemical function of said protein in said subject.

4. The method of claim 3, wherein said modulator comprises one or more antibodies to said protein, or fragments thereof, wherein said antibodies or fragments thereof can inhibit the biochemical function of said protein in said subject.

5. The method of claim 1, wherein said modulator enhances the biochemical function of said protein in said subject.

6. The method of claim 1, wherein said modulator inhibits gene expression of said protein in said subject.

7. The method of claim 6, wherein said modulator comprises any one or more substances selected from the group consisting of antisense oligonucleotides, triple-helix DNA, ribozymes, RNA aptamers, siRNA, double- and single-stranded RNA, wherein said substances are designed to inhibit gene expression of said protein.

8. The method of claim 1, wherein said modulator enhances the gene expression of said protein in said subject.

9. A method to treat, prevent or ameliorate neurodegenerative conditions comprising administering to a subject in need thereof a pharmaceutical composition comprising an effective amount of a modulator of a protein selected from the group consisting of those disclosed in Table 3 or 3A.

10. The method of claim 9, wherein said condition is AD.

11. The method of claim 9, wherein said modulator inhibits the biochemical function of said protein in said subject.

12. The method of claim 11, wherein said modulator comprises one or more antibodies to said protein, or fragments thereof, wherein said antibodies or fragments thereof can inhibit the biochemical function of said protein.

13. The method of claim 9, wherein said modulator enhances the biochemical function of said protein in said subject.

14. The method of claim 9, wherein said modulator inhibits gene expression of said protein in said subject.

15. The method of claim 14, wherein said modulator comprises any one or more substances selected from the group consisting of antisense oligonucleotides, triple-helix DNA, ribozymes, RNA aptamers, siRNA, double- and single-stranded RNA, wherein said substances are designed to inhibit gene expression of said protein.

16. The method of claim 9, wherein said modulator enhances gene expression of said protein in said subject.

17. A method to identify modulators useful to treat, prevent or ameliorate neurodegenerative conditions comprising assaying for the ability of a candidate modulator to modulate the biochemical function of a protein selected from the group consisting of those disclosed in Table 3 or 3A.

18. The method of claim 17, wherein said method further comprises assaying for the ability of an identified modulator to reverse the pathological effects observed in vitro or in vivo models of said conditions.

19. The method of claim 17, wherein said method further comprises assaying for the ability of an identified modulator to reverse the pathological effects observed in clinical studies with subjects with said conditions.

20. The method according to claim 17, wherein said condition is AD.

21. A method to identify modulators useful to treat, prevent or ameliorate neurodegenerative conditions comprising assaying for the ability of a candidate modulator to modulate gene expression of a protein selected from the group consisting of those disclosed in Table 3 or 3A.

22. The method according to claim 21, wherein said method further comprises assaying for the ability of an identified inhibitory modulator to reverse the pathological effects observed in vitro or in vivo models of said condition.

23. The method according to claim 21, wherein said method further comprises assaying for the ability of an identified inhibitory modulator to reverse the pathological effects observed in clinical studies with subjects with said condition.

24. The method according to claim 21, wherein said condition is AD.

25. A pharmaceutical composition comprising a modulator to a protein selected from the group consisting of those disclosed in Table 3 or 3A in an amount effective to treat, prevent or ameliorate neurodegenerative conditions in a subject in need thereof.

26. The pharmaceutical composition according to claim 25, wherein said condition is AD.

27. The pharmaceutical composition according to claim 25, wherein said modulator inhibits the biochemical function of said protein.

28. The pharmaceutical composition of claim 25, wherein said modulator comprises one or more antibodies to said protein, or fragments thereof, wherein said antibodies or fragments thereof can inhibit the biochemical function of said protein.

29. The pharmaceutical composition according to claim 25, wherein said modulator enhances the biochemical function of said protein.

30. The pharmaceutical composition according to claim 25, wherein said modulator inhibits gene expression of said protein.

31. The pharmaceutical composition of claim 30, wherein said modulator comprises any one or more substances selected from the group consisting of antisense oligonucleotides, triple-helix DNA, ribozymes, RNA aptamer, siRNA, double- and single-stranded RNA, wherein said substances are designed to inhibit gene expression of said protein.

32. The pharmaceutical composition according to claim 25, wherein said modulator enhances gene expression of said protein.

33. A method to diagnose subjects suffering from neurodegenerative conditions who may be suitable candidates for treatment with modulators to a protein selected from the group consisting of those disclosed in Table 3 or 3A, comprising assaying mRNA levels of any one or more of said proteins in a biological sample from said subject wherein subjects with altered levels compared to controls would be suitable candidates for modulator treatment.

34. A method to diagnose subjects suffering from neurodegenerative conditions who may be suitable candidates for treatment with modulators to a protein selected from the group consisting of those disclosed in Table 3 or 3A, comprising detecting levels of any one or more of said proteins in a biological sample from said subject wherein subjects with altered levels compared to controls would be suitable candidates for modulator treatment.

35. A method to treat, prevent or ameliorate a neurodegenerative comprising:
(a) assaying for mRNA levels of a protein selected from the group consisting of those disclosed in Table 3 or 3A in a subject; and
(b) administering to a subject with altered levels of mRNA of said protein compared to controls a modulator to said protein in an amount sufficient to treat, prevent or ameliorate the pathological effects of said condition.

36. The method of claim 35, wherein said condition is AD.

37. The method of claim 35, wherein said modulator enhances the gene expression of said protein.

38. The method of claim 35, wherein said modulator inhibits the gene expression of said protein.

39. A method to treat, prevent or ameliorate a neurodegenerative condition comprising:
(a) assaying for levels of a protein selected from the group consisting of those disclosed in Table 3 or 3A in a subject; and
(b) administering to a subject with altered levels of said protein compared to controls a modulator to said protein in an amount sufficient to treat, prevent or ameliorate the pathological effects of said condition.

40. The method of claim 39, wherein said condition is AD.

41. The method of claim 39, wherein said modulator enhances the biochemical function of said protein.

42. The method of claim 39, wherein said modulator inhibits the biochemical function of said protein.

43. A diagnostic kit for detecting mRNA levels of a protein selected from the group consisting of those disclosed in Table 3 or 3A in a biological sample, said kit comprising:
(a) a polynucleotide of a polypeptide set forth in Table 3 or 3A or a fragment thereof;
(b) a nucleotide sequence complementary to that of (a);
(c) a polypeptide of Table 3 or 3A of the present invention encoded by the polynucleotide of (a);
(d) an antibody to the polypeptide of (c);
(e) an RNAi sequence complementary to that of (a),
wherein components (a), (b), (c), (d) or (e) may comprise a substantial component.

44. A diagnostic kit for detecting levels of a protein selected from the group consisting of those disclosed in Table 3 or 3A in a biological sample, said kit comprising:
(a) a polynucleotide of a polypeptide set forth in Table 3 or 3A or a fragment thereof;
(b) a nucleotide sequence complementary to that of (a);
(c) a polypeptide of Table 3 or 3A of the present invention encoded by the polynucleotide of (a);
(d) an antibody to the polypeptide of (c);
(e) an RNAi sequence complementary to that of (a),
wherein components (a), (b), (c), (d) or (e) may comprise a substantial component.
